(19) United States
(12) Patent Application Publication
(10) Pub. No.: US 2002/0193389 A1
(43) Pub. Date: Dec. 19, 2002

(54) Method of inhibiting neoplastic cells with imidazoquinazoline derivatives

(76) Inventors: Rifat Pamukeu, Spring House, PA (US); Gary A. Piazza, Doylestown, PA (US)

Correspondence Address: Cell Pathways, Inc., 702 Electronic Drive, Horsham, PA, 19044, US

(21) Appl. No.: 10/158,455

(22) Filed: May 30, 2002

Related U.S. Application Data
(63) Continuation of application No. 09/196,206, filed on Nov. 20, 1998, now abandoned.

(51) Int. Cl. A61K 31/517

(52) U.S. Cl. 514/267

(57) ABSTRACT

A method for inhibiting neoplasia, particularly cancerous and precancerous lesions by exposing the affected cells to imidazoquinazoline derivatives.

### TECHNICAL FIELD

This invention relates to a method for the selective inhibition of neoplastic cells, for example, for the treatment or prevention of precancerous lesions or other neoplasias in mammals.

### BACKGROUND OF THE INVENTION

Each year in the United States alone, untold numbers of people develop precancerous lesions, which is a form of neoplasia, as discussed below. Such lesions exhibit a strong tendency to develop into malignant tumors, or cancer. Such lesions include lesions of the breast (that can develop into breast cancer), lesions of the skin (that can develop into malignant melanoma or basal cell carcinoma), colonic adenomatous polyps (that can develop into colon cancer), and other such neoplasms. Compounds that prevent or induce the remission of existing precancerous or cancerous lesions or carcinomas would greatly reduce illness and death from cancer.

For example, approximately 60,000 people die from colon cancer, and over 150,000 new cases of colon cancer are diagnosed each year. For the American population as a whole, individuals have a six percent lifetime risk of developing colon cancer, making it the second most prevalent form of cancer in the country. Colon cancer is also prevalent in Western Europe. It is believed that increased dietary fat consumption is increasing the risk of colon cancer in Japan.

In addition, the incidence of colon cancer reportedly increases with age, particularly after the age of 40. Since the mean ages of populations in America and Western Europe are increasing, the prevalence of colorectal cancer should increase in the future.

To date, little progress has been made in the prevention and treatment of colorectal cancer, as reflected by the lack of change in the five-year survival rate over the last few decades. The only cure for this cancer is surgery at an extremely early stage. Unfortunately, most of these cancers are discovered too late for surgical cure. In many cases, the patient does not experience symptoms until the cancer has progressed to a malignant stage.

In view of these grim statistics, efforts in recent years have concentrated on colon cancer prevention. Colon cancer usually arises from pre-existing benign neoplastic growths known as polyps. Prevention efforts have emphasized the identification and removal of colonic polyps. Polyps are identified by x-ray and/or colonoscopy, and usually removed by devices associated with the colonoscope. The increased use of colon x-rays and colonoscopies in recent years has detected clinically significant precancerous polyps in four to six times the number of individuals per year that acquire colon cancer. During the past five years alone, an estimated 3.5 to 5.5 million people in the United States have been diagnosed with adenomatous colonic polyps, and it is estimated that many more people have or are susceptible to developing this condition, but are as yet undiagnosed. In fact, there are estimates that 10-12 percent of people over the age of 40 will form clinically significant adenomatous polyps.

Removal of polyps has been accomplished either with surgery or fiber-optic endoscopic polypectomy—procedures that are uncomfortable, costly (the cost of a single polypectomy ranges between $1,000 and $1,500 for endoscopic treatment and more for surgery), and involve a small but significant risk of colon perforation. Overall, about $2.5 billion is spent annually in the United States in colon cancer treatment and prevention.

In the breast, breast cancer is often treated surgically, often by radical mastectomy with its painful aftermath. Such surgery is costly, too.

As indicated above, each lesion carries with it a chance that it will develop into a cancer. The likelihood of cancer is diminished if a precancerous lesion is removed. However, many of these patients demonstrate a propensity for developing additional lesions in the future. They must, therefore, be monitored periodically for the rest of their lives for reoccurrence.

In most cases (i.e. the cases of sporadic lesion formation, e.g. so-called common sporadic polyps), lesion removal will be effective to reduce the risk of cancer. In a small percentage of cases (i.e. cases where numerous lesions form, e.g. the so-called polyposis syndromes), removal of all or part of the effected area (e.g. the colon) is indicated. For example, the difference between common sporadic polyps and polyposis syndromes is dramatic. Common sporadic polyp cases are characterized by relatively few polyps which can usually be removed leaving the colon intact. By contrast, polyposis syndrome cases can be characterized by many (e.g. hundreds or more) of polyps—literally covering the colon in some cases—making safe removal of the polyps impossible short of surgical removal of the colon.

Because each lesion carries with it a palpable risk of cancerous development, patients who form many lesions (e.g. polyposis syndrome patients) invariably develop cancer if left untreated. Surgical removal of the colon is the conventional treatment in polyposis patients. Many polyposis patients have undergone a severe change in lifestyle as a result of the disfiguring surgery. Patients have strict dietary restrictions, and many must wear ostomy appliances to collect their intestinal wastes.

The search for drugs useful for treating and preventing cancer is intensive. Indeed, much of the focus of cancer research today is on the prevention of cancer because chemotherapy for cancer itself is often not effective and has severe side effects. Cancer chemoprevention is important for recovered cancer patients who retain a risk of cancer reoccurrence. Also, cancer prevention is important for people who have not yet had cancer, but have hereditary factors that place them at risk of developing cancer. With the development of new genetic screening technologies, it is easier to identify those patients with high-risk genetic factors, such as the potential for polyposis syndrome, who would greatly benefit from chemopreventative drugs. Therefore, finding such anti-cancer drugs that can be used for prolonged preventive use is of vital interest.

Known chemopreventative and chemotherapeutic drugs are believed to kill cancer cells by inducing apoptosis, sometimes referred to as “programmed cell death.” Apoptosis naturally occurs in virtually all tissues of the body, and especially in self-renewing tissues such as bone marrow, immune cells, gut, liver and skin. Apoptosis plays a critical role in tissue homeostasis, that is, it ensures that the number of new cells produced are correspondingly offset by an equal number of cells that die. For example, the cells in the intestinal lining divide so rapidly that the body must eliminate cells after only three days in order to prevent the overgrowth of the intestinal lining.

Recently, scientists have realized that abnormalities of apoptosis can lead to the formation of precancerous lesions and carcinomas. Also, recent research indicates that defects in apoptosis play a major role in other diseases in addition to cancer. Consequently, compounds that modulate apoptosis could be used to prevent or control cancer, as well as used in the treatment of other diseases.

Unfortunately, even though known chemotherapeutic drugs may exhibit such desirable apoptosis effects, most chemotherapeutic drugs have serious side effects that prohibit their long-term use, or use in otherwise healthy individuals with precancerous lesions. These side effects, which are a result of the high levels of cytotoxicity of the drugs, include hair loss, weight loss, vomiting, immune suppression and other toxicities. Therefore, there is a need to identify new drug candidates for therapy that do not have such serious side effects in humans.

In recent years, several non-steroidal anti-inflammatory drugs (“NSAIDs”), originally developed to treat arthritis, have shown effectiveness in inhibiting and eliminating colonic polyps. Polyps virtually disappear when the patients take the drug, particularly when the NSAID sulindac is administered. However, the prophylactic use of currently available NSAIDs, even in polyposis syndrome patients, is marked by severe side reactions that include gastrointestinal irritations, perforations, ulcerations and kidney toxicity. Once NSAID treatment is terminated due to such complications, the polyps return, particularly in polyposis syndrome patients.

Sulindac has been particularly well received among the NSAIDs for polyp treatment. Sulindac is a sulfoxide compound that itself is believed to be inactive as an anti-arthritic agent. The sulfoxide is reportedly converted by liver enzymes to the corresponding sulfide, which is acknowledged to be the active moiety as a prostaglandin synthesis inhibitor. The sulfide, however, is associated with the side effects of conventional NSAIDs. The sulfoxide is also known to be metabolized to a sulfone compound that has been found to be inactive as an inhibitor of prostaglandin synthesis but active as an inhibitor of precancerous lesions.

### SUMMARY OF THE INVENTION

This invention includes a method of inhibiting neoplastic cells by exposing those cells to a pharmacologically effective amount of those compounds described below. Such compounds are effective in modulating apoptosis and eliminating and inhibiting the growth of neoplasias such as precancerous lesions, but are not characterized by the severe side reactions of conventional NSAIDs or other chemotherapeutics.

The compounds of that are useful in the methods of this invention include those of Formula I: or pharmacologically acceptable salts thereof,

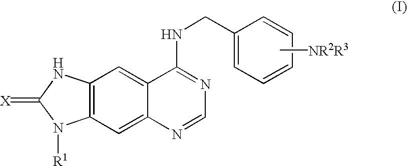

wherein

R′ is selected from the group consisting of hydrogen, a substituted or unsubstituted lower alkyl, a substituted or unsubstituted cycloalkyl, a substituted or unsubstituted bicycloalkyl, a substituted or unsubstituted tricycloalkyl, a substituted or unsubstituted benzocycloalkenyl, a substituted or unsubstituted lower alkenyl, a substituted or unsubstituted aralkyl group, a substituted or unsubstituted aryl group, or a substituted or unsubstituted hetero-aryl group;

R² is selected from the group consisting of hydrogen, a substituted or unsubstituted lower alkyl, a substituted or unsubstituted cycloalkyl, a substituted or unsubstituted bicycloalkyl, a substituted or unsubstituted tricycloalkyl, a substituted or unsubstituted benzocycloalkenyl, a substituted or unsubstituted lower alkenyl, a substituted or unsubstituted aralkyl group, a substituted or unsubstituted aryl group, or a substituted or unsubstituted heteroaryl group;

R³ is selected from the group consisting of hydrogen, a substituted or unsubstituted lower alkyl, a substituted or unsubstituted cycloalkyl, a substituted or unsubstituted bicycloalkyl, a substituted or unsubstituted tricycloalkyl, a substituted or unsubstituted lower alkenyl, a substituted or unsubstituted aralkyl, a substituted or unsubstituted aryl group or a substituted or unsubstituted hetero-aryl group; or R² and R³ may, together with N to which they are attached, form a substituted or unsubstituted heterocyclic group; and

X represents O or S.

### DETAILED DESCRIPTION OF THE PREFERRED EMBODIMENTS

As indicated above, this invention relates to a method for inhibiting neoplasia, particularly cancerous and precancerous lesions by exposing the affected cells to a compound of Formula I above.

Preferably, such compounds are administered without therapeutic amounts of an NSAID.

The present invention is also a method of treating mammals with precancerous lesions by administering a pharmacologically effective amount of an enterically coated pharmaceutical composition that includes compounds of Formula I.

Also, the present invention is a method of inhibiting the growth of neoplastic cells by exposing the cells to an effective amount of compounds of Formula I, wherein R₁ through R₃ etc. are defined as above.

In still another form, the invention is a method of inducing apoptosis in human cells by exposing those cells to an effective amount of compounds of Formula I to those cells sensitive to such a compound.

As used herein, the term “precancerous lesion” includes syndromes represented by abnormal neoplastic, including dysplastic, changes of tissue.

Examples include adenomatous growths in colonic, breast or lung tissues, or conditions such as dysplastic nevus syndrome, a precursor to malignant melanoma of the skin. Examples also include, in addition to dysplastic nevus syndromes, polyposis syndromes, colonic polyps, precancerous lesions of the cervix (i.e., cervical dysplasia), prostatic dysplasia, bronchial dysplasia, breast, bladder and/or skin and related conditions (e.g., actinic keratosis), whether the lesions are clinically identifiable or not.

As used herein, the term “carcinomas” refers to lesions that are cancerous. Examples include malignant melanomas, breast cancer, and colon cancer.

As used herein, the term “neoplasm” refers to both precancerous and cancerous lesions.

It will also be appreciated that a compound of Formula I or a physiologically acceptable salt or solvate thereof can be administered as the raw compound, or as a pharmaceutical composition containing either entity.

Compounds useful in the methods of this invention are preferably formulated into compositions together with pharmaceutically acceptable carriers for oral administration in solid or liquid form, or for rectal administration, although carriers for oral administration are most preferred.

Pharmaceutically acceptable carriers for oral administration include capsules, tablets, pills, powders, troches and granules. In such solid dosage forms, the carrier can comprise at least one inert diluent such as sucrose, lactose or starch. Such carriers can also comprise, as is normal practice, additional substances other than diluents, e.g., lubricating agents such as magnesium stearate. In the case of capsules, tablets, troches and pills, the carriers may also comprise buffering agents. Carriers such as tablets, pills and granules can be prepared with enteric coatings on the surfaces of the tablets, pills or granules. Alternatively, the enterically coated compound can be pressed into a tablet, pill, or granule, and the tablet, pill or granules for administration to the patient. Preferred enteric coatings include those that dissolve or disintegrate at colonic pH such as shellac or Eudraget S.

Pharmaceutically acceptable carriers include liquid dosage forms for oral administration, e.g. pharmaceutically acceptable emulsions, solutions, suspensions, syrups and elixirs containing inert diluents commonly used in the art, such as water. Besides such inert diluents, compositions can also include adjuvants such as wetting agents, emulsifying and suspending agents, and sweetening, flavoring and perfuming agents.

Pharmaceutically acceptable carriers for rectal administration are preferably suppositories that may contain, in addition to the compounds of Formula I, excipients such as cocoa butter or a suppository wax.

The pharmaceutically acceptable carrier and compounds of this invention are formulated into unit dosage forms for administration to a patient. The dosage levels of active ingredient (i.e. compounds of this invention) in the unit dosage may be varied so as to obtain an amount of active ingredient effective to achieve lesion-eliminating activity in accordance with the desired method of administration (i.e., oral or rectal). The selected dosage level therefore depends upon the nature of the active compound administered, the route of administration, the desired duration of treatment, and other factors. If desired, the unit dosage may be such that the daily requirement for active compound is in one dose, or divided among multiple doses for administration, e.g., two to four times per day.

The pharmaceutical compositions of this invention are preferably packaged in a container (e.g. a box or bottle, or both) with suitable printed material (e.g. a package insert) containing indications, directions for use, etc.

The amount and frequency of administration may vary depending on the medication form, patient's age, patient's weight, and patient's conditions. Normally, 0.05˜5 g/60 kg/day would be appropriate in an oral dosage form. As for intravensous administration, it is desirable not to exceed the daily amount in a oral form, which should be administered at a rate of 0.01˜5 mg/kg/minute.

The term “lower alkyl” as used herein is straight or branched and has from 1 to about 8 carbon atoms. Examples of a lower alkyl include methyl, ethyl, propyl, isopropyl, butyl, isobutyl, sec-butyl, tert-butyl, pentyl, isopentyl, neo-pentyl, sec-pentyl, tert-pentyl, hexyl, isohexyl, heptyl, octyl, iso-octyl and so on. A “cycloalkyl” as used herein has 3 to about 8 carbon atoms. Examples of a cycloalkyl include cyclopropyl, cyclobutyl, cyclopentyl, cyclohexyl, cycloheptyl, cyclooctyl and so on. A “bicycloalkyl” as used herein has 7 to 10 carbon atoms. Examples include bicyclo[2,2,1]heptyl, bicyclo[2,2,2]octyl, bicyclo[3,3,1]nonyl, and so on. A “tricycloalkyl” as used herein has 9 to about 12 carbon atoms. Examples include tricyclo[3,3,1,1^{3,7}]decyl, tricyclo[3,3,1,0^{3,7}]nonyl, tricyclo[5,4,0,0^{2,9}]undecyl, and so on. A benzocycloalkenyl has 8 to about 12 carbon atoms. Examples include benzocyclobutenyl, indanyl, benzocyclooctenyl and so forth. A “lower alkenyl” as used herein is straight or branched and has 2 to about 6 carbon atoms. Examples of a lower alkenyl include vinyl, aryl, propenyl, methacryl, butenyl, crotyl, pentenyl, hexenyl and so on. An “aralkyl” has 7˜15 carbon atoms. Examples include benzyl, phenethyl, and benzhydryl, naphthylmethyl and so on. Examples of an aryl include phenyl, naphthyl and so on. Examples of heteroaryl include pyridyl, quinolyl, isoquinolyl, thienyl, furil, pyrrolyl, benzothienyl, benzofuril, indolyl, tetrahydroquinolyl, tetrahydroisoquinolyl, imidazolinyl and so on. Examples of a lower alkyl having hydroxy groups include the aforementioned lower alkyl groups which are substituted with 1˜3 hydroxy groups, such as hydroxymethyl, 2-hydroxyethyl, 3-hydroxypropyl, 4-hydroxybutyl, 3-hydroxybutyl, 2-hydroxybutyl, 5-hydroxypentyl and so on. Examples of heterocyclic groups formed by including N include pyrrolidinyl, piperidino, piperazinyl, morpholino, thiomorpholino, homopiperazinyl, imidazolyl, 1-perhydroazevinyl, 1-perhydroazocinyl, tetrahydroisoquinolyl and so on.

Substituents on a substituted lower alkyl, a substituted cycloalkyl, a substituted bicycloalkyl, and a substituted tricycloalkyl are 1 to about 3 of the same or different groups, whose examples include the following: cycloalkyl, hydroxy, a lower alkoxy, a hydroxyalkoxy, carboxy, a lower alkoxycarbonyl, amino, a monoalkyl-substituted amino, a dialkyl-substituted amino, a monoaryl-substituted amino, a diaryl-substituted amino, nitro, halogeno, a substituted or unsubstituted heteroaryl, a substituted or unsubstituted alicyclic heterocyclic group and so on.

The alkyl portions of a lower alkoxy, a lower alkoxycarbonyl, monoalkyl-substituted amino and dialkyl-substituted amino are defined the same as the previously mentioned lower alkyl groups. The aryl portions of a monoaryl-substituted amino and a diaryl-substituted amino are the same as the previously defined aryl groups.

“Halogen” means an atom of fluorine, chlorine, bromine or iodine.

Examples of an alicyclic heterocyclic group include tetrahydrofuril, piperidino, piperidyl, morpholino, morpholinyl, thiomorpholino, thiomorpholinyl, piperazinyl, homopiperazinyl, pyrrolidinyl, imidazolyl, tetrahydroisoquinolyl and so on. Substituents on a substituted alicyclic heterocycle include a lower alkyl, aryl or aralkyl.

Examples of a substituted alicyclic heterocyclic group include N-methylpiperazinyl, N-ethylpiperazinyl, N-methylhomopiperazinyl, N-phenylpiperazinyl, N-benzylpiperazinyl and so on.

Substituents on a substituted benzocycloalkenyl, a substituted lower alkenyl, a substituted aralkyl, a substituted aryl, a substituted heteroaryl and, a substituted heterocyclic group formed by including N are 1 to about 5 of the same or different of the following groups: a lower alkyl, hydroxy, a lower hydroxy alkyl, a lower alkoxy, a lower alkoxyalkyl, carboxy, a lower alkoxycarbonyl, amino, a monoalkyl-substituted amino, a dialkyl-substituted amino, nitro, sulfonamido, halogeno, trifluoromethyl, and so on. The alkyl portions of a lower alkyl, a lower alkoxy, a lower alkoxyalkyl, a lower alkoxycarbonyl, a monoalkyl-substituted amino and a dialkyl-substituted amino are defined the same as the previously defined lower alkyl groups.

Preferred examples of a substituted heterocyclic group formed by including N include N-methylpiperazinyl, N-ethylpiperazinyl, N-methylhomopiperazinyl, N-phenylpiperazinyl, N-benzylpiperazinyl, N-benzylpiperidino and so on.

Pharmacologically acceptable salts of Compounds (I) include pharmacologically acceptable acid salts. Examples include inorganic acid salts such as hydrochloride, hydrobromide, hydroiodide nitrate, and sulfate, and organic acid salts such as formate, acetate, benzoate, tartarate, maleate, fumarate, succinate, oxalate, glyoxylate, asparagate, methanesulfonate, and benzenesulfonate.

Compounds of Formula I may be prepared by any suitable method known in the art or by the following processes disclosed in WO98/08848 which is incorporated herein by reference. In the methods below, R¹ and R² etc. are as defined in Formula I above unless otherwise indicated.

Production Method 1-1

The source material (VII) for the production of Compounds (I) can be produced according to the following reaction process.

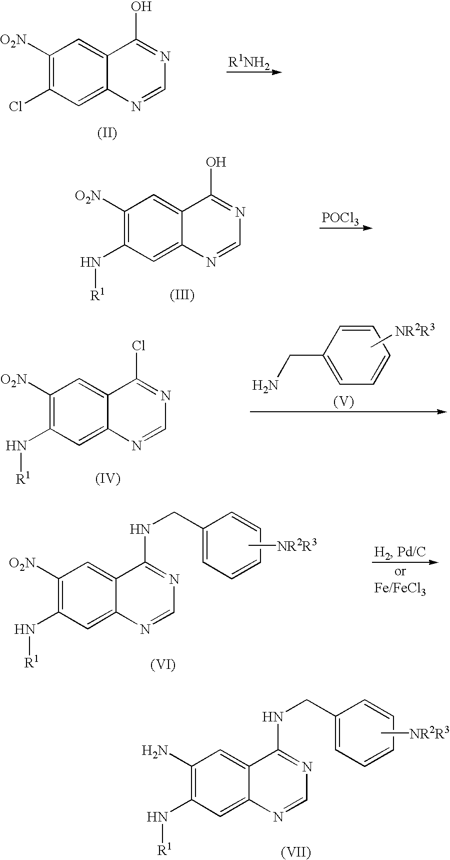

(In the formula, R¹, R² and R³ are the same as previously described.)

The source material (II) can be obtained by following the known method as detailed in the “Journal of Organic Chemistry, Vol.40, p356 (1975)”, and so on.

The compound (III) can be obtained by reacting its source material with the equivalent or excess amount of the amine expressed by the formula R¹NH₂ (in the formula, R¹ has the same definition as previously described) or its aqueous solution in a solvent such as ethanol, butanol, or dimethylsulfoxide, which is placed in a sealed vessel (sealed tube) as necessary, at a temperature between room temperature and 150 degrees Celsius for 1˜24 hours.

The compound (IV) can be obtained by reacting compound (III) and a chlorinating agent such as phosphorus oxychloride, thionyl chloride, and phosphorus pentachloride, as necessary in the presence of a base such as triethylamine, N,N-diisopropyl thylamine and pyridine, for 1˜24 hours. The reaction may be performed without a solvent, or with a solvent such as dichloromethane or dichloroethane. Furthermore, N,N-dimethylformamide can be added. When a solvent is used, the reaction is performed at a temperature between room temperature and the boiling point of the solvent in use. When a solvent is not used, the reaction is performed at a temperature between room temperature and the boiling point of the chlorinating agent in use.

The compound (VI) can be obtained by reacting the compound (IV) with the equivalent or excess amount of the amine represented by Formula (V) (in the formula, R² and R³ have the same definitions as before), as necessary in the presence of 3˜10 equivalence of a base such as triethylamine, in a solvent such as tetrahydrofuran at a temperature between room temperature and the boiling point of the solvent in use for 30 minutes to 24 hours. Moreover, the source material (V) can be obtained through a method described in the reference examples or through a similar method. Further, the section NR²R³ in Formula (VI) can be protected by an appropriate protecting group before using it for the following reduction reaction.

The compound (VII) can be obtained by a catalytic reduction of the compound (VI) under the presence of a catalytic reduction catalysis such as palladium carbon in a solvent such as water, tetrahydrofuran, methanol, ethanol, or NN-dimethylformamide. This can be done under a hydrogen environment or a hydrogen flow at a temperature between room temperature and the boiling point of the solvent in use for 3˜24 hours as it is being stirred. The amount of catalytic reduction catalysis is 1/100˜1/10 of the base material in weight. Alternatively, the compound (VII) can be obtained by reducing the compound (VI) under the presence of a catalytic reduction agent such as iron/iron dichloride (iron dichloride, 1/100˜1/20 of the base material in weight, is added to 1˜4 equivalence of reduced ion). This is done in a solvent such as ethanol hydrate or water at a temperature between room temperature and the boiling temperature of the solvent in use for 1˜10 hours as it is being stirred.

Production Method 1-2

Compound (Ia) is one of Compounds (I) in which X is O (oxygen). Compound (Ia) can be produced by the following reaction process which uses the compound (VII) as a source material.

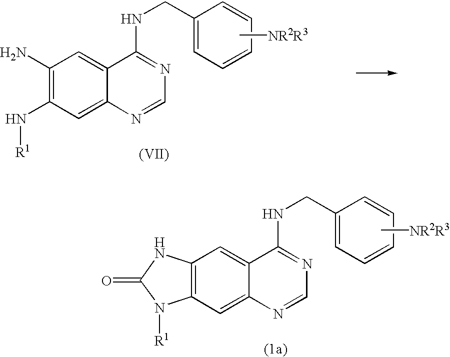

(In the formula, R¹, R² and R³ are the same as previously described.)

Compound (Ia) can be obtained by reacting and cyclizing compound (VII). The reaction and cyclization occur with 1˜10 equivalence of a carbonylation reagent such as N,N′-carbonyldiimidazole, phosgene, urea, alkyl chlorocarbonate, and aryl chlorocarbonate, as necessary under the presence of 1˜10 equivalence of a base, in an inert solvent. Examples of a base include triethylamine, pyridine and so on. Examples of an inert solvent include water, alcohol (methanol, ethanol and so on), a non-polar solvent (ethyl acetate, ether and so on), a aprotic polar solvent (acetonitrile, N,N-dimethylformamide, N,N-dimethylacetoamide, demethylsulfoxide, tetrahydrofuran, dioxane and so on. The reaction is performed at a temperature between 0 degrees Celsius and the boiling point of the solvent in use, and is completed in 10 minutes˜48 hours.

When the portion NR²R³ in compound (VI) is protected by an appropriate protecting group, the protecting group can be removed after the reduction reaction in Production Method 1-1, or after the carbonylation reaction in Production Method 1-2, in order to obtain the desired compound.

Production Method 1-3

Compound (Ia) is Compound (I) in which X is O (oxygen). Compound (Ia) can be produced by the following reaction process which uses the compound (VII) as a source material.

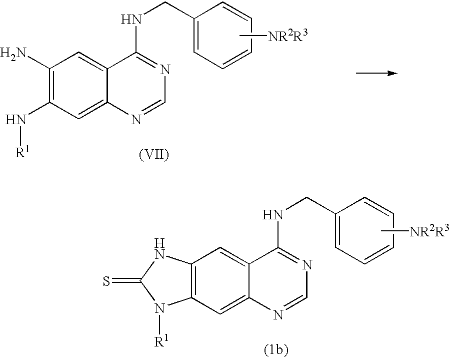

(In the formulae, R¹, R¹ and R³ are the same as previously described.)

Compound (Ib) can be obtained by reacting the compound (VII) with a thiocarbonilation reagent such as 1˜10 equivalence of N,N′-thiocarbonyldiimidazole, thiophosgene, or 10˜200 equivalence of carbon disulfide, as necessary under the presence of 1˜10 equivalence of a base, in an inert solvent, and cyclizing it. Examples of a base and an inert solvent include those listed for Production Method 1-2, in which Compound (Ia) is produced. The reaction is performed at a temperature between 0 degrees Celsius and the boiling point of the solvent in use, and is completed in 10 minutes˜48 hours.

When the portion NR²R³ in the compound (VI) is protected by an appropriate protecting group, the protecting group can be removed after the reduction reaction in Production Method 1-1, or after the thiocarbonylation reaction in Production Method 1-3, in order to obtain the desired compound.

Intermediate compounds and target compounds in the above production methods can be isolated and purified by utilizing typical purification methods in use in the organic chemistry syntheses. Examples of such typical purification methods include filtration, extraction, washing, drying, condensation, re-crystallization, various chromatography methods, and so on. Furthermore, intermediate compounds may be used for a subsequent reaction without a particular purification process.

Position isomers, geometrical isomers, optical isomers or tautomers may exist in some of Compounds (I). In addition to these, the present invention includes all possible isomers and mixtures thereof.

For obtaining salts of Compounds (I), if Compounds (I) manifest themselves in a salt form, then the salt can be purified without modification. If Compounds (I) manifest themselves in a free form, then they can be dissolved or suspended in an appropriate solvent, and then an acid can be added. Thus, resultant salts can be isolated and purified.

Moreover, Compounds (I) and pharmacologically acceptable salts thereof may exist in a form of adduct with water or other solvents. These adducts are also included in the present invention.

Examples of compounds of Formula (I), which are useful in the practice of the present invention, are shown in Table 1.

TABLE 1-1

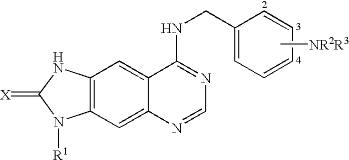

| | | | Sub- | | |
|---|---|---|---|---|---|
| Com- | | | stitution | | |
| pound | | | Position | | |
| Number | X | R¹ | (2, 3, 4) | R² | R³ |
| 1 | O | CH₂CH₃ | 2 | H | CH₃ |
| 2 | O | CH₂CH₃ | 4 | H | CH₃ |
| 3 | O | CH₂CH₃ | 4 | H | 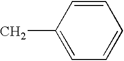 |
| 4 | O | CH₂CH₃ | 4 | H | CH(CH₃)₂ |
| 5 | O | CH₂CH₃ | 4 | H | CH₂CH₂CH₃ |
| 6 | O | CH₂CH₃ | 4 | H | CH₂CH₃ |
| 7 | O | CH₂CH₃ | 2 | H | 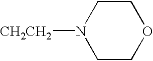 |
| 8 | O | CH₂CH₃ | 2 | H | 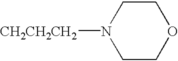 |
| 9 | O | CH₂CH₃ | 2 | H | CH₂CH₂OH |
| 10 | O | CH₂CH₃ | 2 | H | CH₂CH₂OCH₃ |

TABLE 1-2

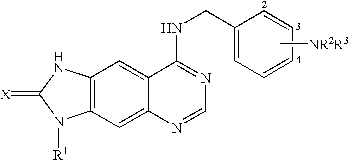

| | | | Sub- | | |
|---|---|---|---|---|---|
| Com- | | | stitution | | |
| pound | | | Position | | |
| Number | X | R¹ | (2, 3, 4) | R² | R³ |

| | | | | |
|---|---|---|---|---|
| 11 | O | CH₂CH₃ | 2 | —CH₂CH₂—O—CH₂CH₂— |
| 12 | O | CH₂CH₃ | 2 | 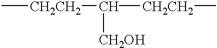 |
| 13 | O | CH₂CH₃ | 2 | —CH₂CH₂—CH₂—CH₂CH₂— |
| 14 | O | CH₂CH₃ | 2 | —CH₂CH₂—N(CH₃)—CH₂CH₂— |
| 15 | O | CH₂CH₃ | 2 | —CH₂CH₂—S—CH₂CH₂— |
| 16 | O | CH₂CH₃ | 3 | —CH₂CH₂—O—CH₂CH₂— |
| 17 | O | CH₂CH₃ | 3 | —CH₂CH₂—CH₂—CH₂CH₂— |
| 18 | O | CH₂CH₃ | 3 | —CH₂CH₂—N(CH₃)—CH₂CH₂— |
| 19 | O | CH₂CH₃ | 4 | —CH₂CH₂—CH₂—CH₂CH₂— |
| 20 | O | CH₂CH₃ | 4 | —CH₂CH₂—N(CH₃)—CH₂CH₂— |

TABLE 1-3

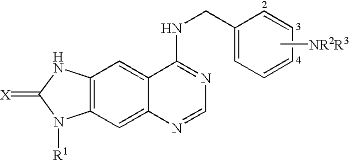

| | | | Substitution | | |
|---|---|---|---|---|---|
| Compound | | | Position | | |
| Number | X | R¹ | (2, 3, 4) | R² | R³ |
| 21 | O | CH₂CH₃ | 4 | —CH₂CH₂—S—CH₂CH₂— | |
| 22 | S | CH₂CH₃ | 2 | H | CH₃ |
| 23 | S | CH₂CH₃ | 4 | H | CH₃ |
| 24 | S | CH₂CH₃ | 4 | H | 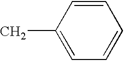 |
| 25 | S | CH₂CH₃ | 4 | H | CH(CH₃)₂ |
| 26 | S | CH₂CH₃ | 4 | H | CH₂CH₂CH₃ |
| 27 | S | CH₂CH₃ | 4 | H | CH₂CH₃ |
| 28 | S | CH₂CH₃ | 2 | H | 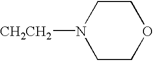 |
| 29 | S | CH₂CH₃ | 2 | H | 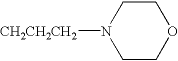 |
| 30 | S | CH₂CH₃ | 2 | H | CH₂CH₂OH |

TABLE 1-4

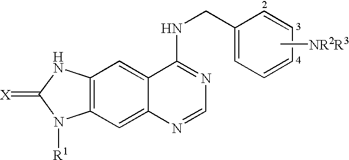

| | | | Sub- | | |
|---|---|---|---|---|---|
| Com- | | | stitution | | |
| pound | | | Position | | |
| Number | X | R¹ | (2, 3, 4) | R² | R³ |
| 31 | S | CH₂CH₃ | 4 | H | CH₂CH₂OH |
| 32 | S | CH₂CH₃ | 2 | H | CH₂CH₂OCH₃ |

| | | | | |
|---|---|---|---|---|
| 33 | S | CH₂CH₃ | 2 | —CH₂CH₂—O—CH₂CH₂— |
| 34 | S | CH₂CH₃ | 2 | 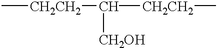 |
| 35 | S | CH₂CH₃ | 2 | —CH₂CH₂—CH₂—CH₂CH₂— |
| 36 | S | CH₂CH₃ | 2 | —CH₂CH₂—N(CH₃)—CH₂CH₂— |
| 37 | S | CH₂CH₃ | 2 | —CH₂CH₂—S—CH₂CH₂— |
| 38 | S | CH₂CH₃ | 3 | —CH₂CH₂—O—CH₂CH₂— |
| 39 | S | CH₂CH₃ | 3 | —CH₂CH₂—CH₂—CH₂CH₂— |
| 40 | S | CH₂CH₃ | 3 | —CH₂CH₂—N(CH₃)—CH₂CH₂— |

TABLE 1-5

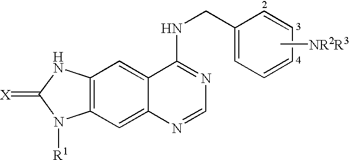

| | | | Sub- | | |
|---|---|---|---|---|---|
| Com- | | | stitution | | |
| pound | | | Position | | |
| Number | X | R¹ | (2, 3, 4) | R² | R³ |
| 41 | S | CH₂CH₃ | 4 | —CH₂CH₂—O—CH₂CH₂— | |
| 42 | S | CH₂CH₃ | 4 | —CH₂CH₂—CH₂—CH₂CH₂— | |
| 43 | S | CH₂CH₃ | 4 | —CH₂CH₂—N(CH₃)—CH₂CH₂— | |
| 44 | S | CH₂CH₃ | 4 | —CH₂CH₂—CH₂CH₂— | |
| 45 | S | CH₂CH₃ | 4 | —CH₂CH₂—S—CH₂CH₂— | |
| 46 | S | CH₂CH₃ | 2 | CH₃ | CH₂CH₂OH |
| 47 | S | CH₂CH₃ | 4 | 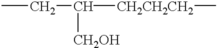 | |
| 48 | O | CH₂CH₃ | 4 | —CH₂CH₂—O—CH₂CH₂— | |
| 49 | O | CH₂CH₃ | 4 | H | CH₂CH₂OH |

| | | | | |
|---|---|---|---|---|
| 50 | O | CH₂CH₃ | 2 | —CH═CH—N═CH— |

TABLE 1-6

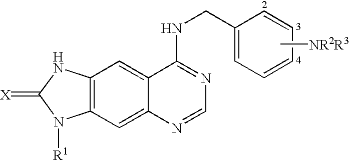

| | | | Substitution | | |
|---|---|---|---|---|---|
| Compound | | | Position | | |
| Number | X | R¹ | (2, 3, 4) | R² | R³ |
| 51 | S | CH₂CH₃ | 2 | —CH═CH—N═CH— | |
| 52 | O | CH₂CH₃ | 2 | —CH₂CH₂—CH₂CH₂CH₂—CH₂CH₂— | |
| 53 | S | CH₂CH₃ | 2 | —CH₂CH₂—CH₂CH₂CH₂—CH₂CH₂— | |
| 54 | O | CH₂CH₃ | 2 | H | —CH₂CH₂CH₃ |
| 55 | S | CH₂CH₃ | 2 | H | —CH₂CH₂CH₃ |
| 56 | S | CH₂CH₃ | 2 | H | CH(CH₃)₂ |
| 57 | O | CH₂CH₃ | 2 | 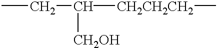 | |
| 58 | S | CH₂CH₃ | 3 | 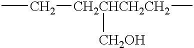 | |
| 59 | S | CH₂CH₃ | 3 | 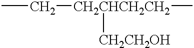 | |
| 60 | O | CH₂CH₃ | 2 | H | CH(CH₃)₂ |

TABLE 1-7

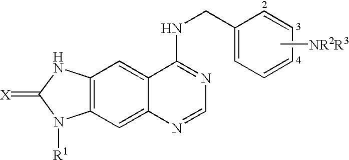

| Com- | | | Substitution | | |
|---|---|---|---|---|---|
| pound | | | Position | | |
| Number | X | R¹ | (2, 3, 4) | R² | R³ |
| 61 | O | CH₂CH₃ | 2 | H | CH₂CH₃ |
| 62 | S | CH₂CH₃ | 2 | H | CH₂CH₃ |
| 63 | S | CH₂CH₃ | 2 | 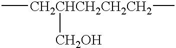 | |
| 64 | S | CH₂CH₃ | 2 | H | 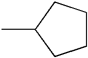 |
| 65 | S | CH₂CH₃ | 2 | H | CH₂CH₂CH₂CH₃ |
| 66 | O | CH₂CH₃ | 2 | —CH₂CH₂CH₂CH₂— | |
| 67 | O | CH₂CH₃ | 2 | H | CH₂CH₂CH₂CH₃ |
| 68 | S | CH₂CH₃ | 2 | H | 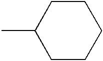 |
| 69 | O | CH₂CH₃ | 2 | H | 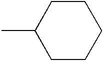 |

| | | | | |
|---|---|---|---|---|
| 70 | S | CH₂CH₃ | 2 | —CH₂CH₂CH₂CH₂— |

TABLE 1-8

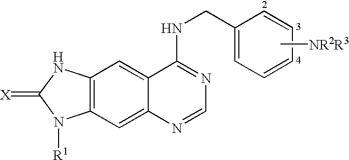

| | | | Substitution | | |
|---|---|---|---|---|---|
| Compound | | | Position | | |
| Number | X | R¹ | (2, 3, 4) | R² | R³ |
| 71 | O | CH₂CH₃ | 2 | —CH₂CH₂—N(CH₂CH₃)—CH₂CH₂— | |
| 72 | O | CH₂CH₃ | 2 | H | CH₂CH(CH₃)₂ |
| 73 | S | CH₂CH₃ | 2 | —CH₂CH₂CH₂CH₂CH₂CH₂— | |
| 74 | O | CH₂CH₃ | 2 | H | 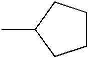 |
| 75 | S | CH₂CH₃ | 2 | H | 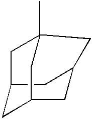 |
| 76 | O | CH₂CH₃ | 2 | 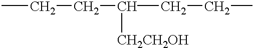 | |
| 77 | S | CH₂CH₃ | 2 | —CH₂CH₂—N(CH₂CH₃)—CH₂CH₂— | |
| 78 | S | CH₂CH₃ | 2 | H | CH₂CH(CH₃)₂ |
| 79 | S | CH₂CH₃ | 2 | H | CH₂CH₂CH₂CH₂OH |

| | | | | |
|---|---|---|---|---|
| 80 | S | CH₂CH₂CH₃ | 2 | 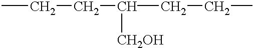 |

TABLE 1-9

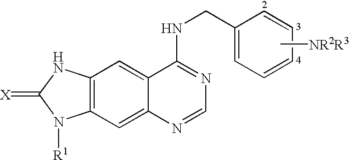

| | | | Substitution | | |
|---|---|---|---|---|---|
| Compound | | | Position | | |
| Number | X | R¹ | (2, 3, 4) | R² | R³ |
| 81 | O | CH₂CH₃ | 2 | —CH₂CH₂—CH₂—CH₂—CH₂CH₂— | |
| 82 | S | CH₂CH₃ | 2 | —CH₂CH₂—N(CH₃)—CH₂—CH₂—CH₂— | |
| 83 | S | CH₂CH₃ | 2 | H | —CH₂CH₂OCH₂CH₂OH |
| 84 | O | CH₂CH₃ | 2 | —CH₂CH₂—N(CH₃)—CH₂—CH₂—CH₂— | |
| 85 | S | CH₃ | 2 | 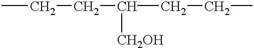 | |
| 86 | S | CH₂CH₃ | 2 | H | 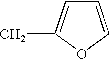 |
| 87 | S | CH₂CH₃ | 2 | 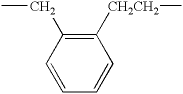 | |
| 88 | S | CH₂CH₃ | 2 | H | 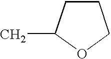 |
| 89 | S | CH₂CH₃ | 2 | H | 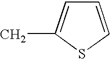 |
| 90 | S | CH₂CH₃ | 2 | H | 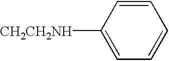 |

TABLE 1-10

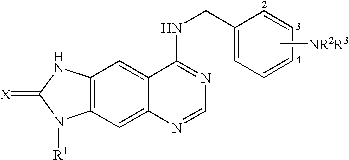

| | | | Substitution | | |
|---|---|---|---|---|---|
| Compound | | | Position | | |
| Number | X | R¹ | (2, 3, 4) | R² | R³ |
| 91 | S | CH₂CH₃ | 2 | 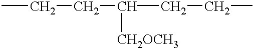 | |
| 92 | S | CH₂CH₃ | 2 | 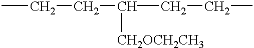 | |
| 93 | S | CH₂CH₃ | 2 | H | H |
| 94 | S | CH₂CH₃ | 2 | H | 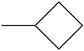 |
| 95 | S | CH₂CH₃ | 2 | H | 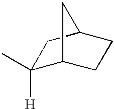 |
| 96 | S | CH₂CH₃ | 2 | —CH₂—CH₂—N(CO₂CH₂CH₃)—CH₂—CH₂— | |
| Control | 20.2 | 19.2 | 3.2 | 3.3 | N = 2 |
| (0.01N HCl) | | | | | |

Reference Examples and Examples disclosed in the aforesaid PCT patent application are used to explain compounds useful in the practice of this invention.

The proton nuclear resonance spectra (NMR) in Examples and Reference Examples are reported at 270 MHz. The peak position is expressed in 1/1.000,000 (ppm) unit from the position for tetramethylsilane to the low magnetic field side. The peak shape is described by the following: “s” stands for a singlet, “d” for a doublet, “t” for a triplet, “m” for a multiplet, and “br” for broad.

### REFERENCE EXAMPLE 1

2-Methylaminobenzonitrile

2-Fluorobenzonitrile (10.0 g, 82.8 millimole) is dissolved in acetonitrile (100 ml), and then 40%-methylamine aqueous solution (200 ml) is added. The solution is stirred for one night at 60 degrees Celsius. In addition, the above methylamine aqueous solution (100 ml) is added, and the solution is stirred for 9 hours at 60 degrees Celsius. The reaction solution is concentrated under reduced pressure, water is added to the obtained residuum, and extraction is performed with chloroform. After the organic layer is dried (magnesium sulfate anhydride), the desiccant is separated through filtration, and the organic layer is concentrated under reduced pressure. The obtained oily material is purified using silica gel chromatography. (At the beginning, it is eluted with chloroform/hexane=1/2. Gradually increasing the contents of the chloroform, at the end, it is eluted with chloroform/hexane=1/1.) Thus, the subject compound (5.38 g, 49%) is obtained as oily material.

¹H-NMR (CDCl₃) δ (ppm): 2.87 (3H, d, J=5.0 Hz), 4.70 (1H, q, J=5.0 Hz), 6.61-6.69 (2H, m), 7.35-7.43 (2H, m).

### REFERENCE EXAMPLE 2

4-Methylaminobenzonitrile

By using the same method as described in Reference Example 1, the subject compound is synthesized from 4-fluorobenzonitrile and a 40%-methylamine aqueous solution.

¹H-NMR (CDCl₃) δ (ppm): 2.87 (3H, d, J=5.0 Hz), 4.39 (1H, br), 6.55 (2H, d, J=8.8 Hz), 7.42 (2H, d, J=8.8 Hz).

### REFERENCE EXAMPLE 3

4-Benzylaminobenzonitrile

4-Fluorobenzonitrile (6.0 g, 49.5 millimole) is dissolved with acetonitrile (60 ml), benzylamine (10.6 g, 98.9 millimole) is added, and the solution is stirred for 3 days at 100 degrees Celsius. In order to complete the reaction, additional benzylamine (21.2 g, 198 millimole) is added, and the solution is stirred for a day at 100 degrees Celsius. The solvent is removed under reduced pressure, water is added to the obtained residuum, and extraction is performed with chloroform. After the organic layer is dried (magnesium sulfate anhydride), the desiccant is separated through filtration, and the filtrate is concentrated under reduced pressure. The obtained oily material is purified using silica gel chromatography (first eluted with chloroform/hexane=1/2, then eluted with chloroform/hexane=1/1 gradually increasing the contents of the chloroform), and the subject compound (7.42 g, 72%) is obtained as oily material.

¹H-NMR (CDCl₃) δ (ppm): 4.32 (2H, d, J=4.7 Hz), 4.80 (1H, br), 6.55 (2H, d, J=8.4 Hz), 7.25-7.37 (7H, m).

### REFERENCE EXAMPLE 4

4-Isopropylaminobenzonitrile

By using the same method as described in Reference Example 3, the subject compound is synthesized from 4-fluorobenzonitrile and isopropylamine.

¹H-NMR (CDCl₃) δ (ppm): 1.23 (6H, d, J=6.6 Hz), 3.61-3.69 (1H, m), 4.22 (1H, br), 6.53 (2H, d, J=8.9 Hz), 7.38 (2H, d, J=8.9 Hz).

### REFERENCE EXAMPLE 5

4-Propylaminobenzonitrile

By using the same method as described in Reference Example 3, the subject compound is synthesized from 4-fluorobenzonitrile and n-propylamine.

¹H-NMR (CDCl₃) δ (ppm): 0.99 (3H, t, J=7.4 Hz), 1.57-1.71 (2H, m), 3.06-3.34 (2H, m), 4.50 (1H, br), 6.55 (2H, d, J=8.7 Hz), 7.38 (2H, d, J=8.7 Hz).

### REFERENCE EXAMPLE 6

4-Ethylaminobenzonitrile

By using the same method as described in Reference Example 1, the subject compound is synthesized from 4-fluorobenzonitrile and a 70%-ethylamine aqueous solution.

¹H-NMR (CDCl₃) δ (ppm): 1.27 (3H, t, J=7.3 Hz), 3.13-3.23 (2H, m), 4.22 (1H, br), 6.54 (2H, d, J=8.6 Hz), 7.40 (2H, d, J=8.6 Hz).

### REFERENCE EXAMPLE 7

2-(2-Morpholinoethylamino)benzonitrile By using the same method as described in Reference Example 3, the subject compound is synthesized from 2-fluorobenzonitrile and 4-(2-aminoethyl)morpholine.

¹H-NMR (CDCl₃) δ (ppm): 2.48-2.56 (4H, m), 2.68 (2H, t, J=6.2 Hz), 3.20 3.27 (2H, m), 3.73-3.80 (4H, m), 5.36 (1H, br), 6.62-6.70 (2H, m), 7.35-7.41 (2H, m).

### REFERENCE EXAMPLE 8

2-(3-Morpholinopropylamino)benzonitrile By using the same method as described in Reference Example 3, the subject compound is synthesized from 2-fluorobenzonitrile and 4-(3-aminopropyl)morpholine.

¹H-NMR (CDCl₃) δ (ppm): 1.79-1.92 (2H, m), 2.46-2.55 (6H, m), 3.22-3.35 (2H, m), 3.75-3.86 (4H, m), 5.67 (1H, br), 6.61-6.70 (2H, m), 7.34-7.39 (2H, m).

### REFERENCE EXAMPLE 9

2-(2-Hydroxyethylamino)benzonitrile

By using the same method as described in Reference Example 3, the subject compound is synthesized from 2-fluorobenzonitrile and ethanolamine.

¹H-NMR (CDCl₃) δ (ppm): 3.30-3.36 (2H, m), 3.43 (1H, br), 3.80-3.85 (2H, m), 5.02 (1H, t, J=5.0 Hz), 6.60-6.67 (2H, m), 7.31-7.36 (2H, m).

### REFERENCE EXAMPLE 10

4-(2-Hydroxyethylamino)benzonitrile By using the same method as described in Reference Example 3, the subject compound is synthesized from 4-fluorobenzonitrile and ethanolamine.

¹H-NMR (CDCl₃—CD₃OD) δ (ppm): 3.26-3.32 (2H, m), 3.78-3.82 (2H, m), 6.58 (2H, d, J=8.9 Hz), 7.39 (2H, d, J=8.9 Hz).

### REFERENCE EXAMPLE 11

2-(2-Methoxyethylamino)benzonitrile By using the same method as described in Reference Example 3, the subject compound is synthesized from 2-fluorobenzonitrile and 2-methoxyethylamine.

¹H-NMR (CDCl₃) δ (ppm): 3.38 (2H, t, J=5.4 Hz), 3.41 (3H, s), 3.62 (2H, t, J=5.4 Hz), 4.86 (1H, br), 6.65-6.70 (2H, m), 7.35-7.40 (2H, m).

### REFERENCE EXAMPLE 12

2-Morpholinobenzonitrile

2-Fluorobenzonitrile (1.21 g, 1.00 millimole) is dissolved with acetonitrile (50 ml), morpholine (33 ml, 377 millimole) is added, and the solution is stirred for 2 nights at 110 degrees Celsius. After completion of the reaction, the reaction solution is concentrated, the obtained oily material is purified using silica gel chromatography (eluted with chloroform), and the subject compound (1.80 g, 95%) is obtained as oily material.

¹H-NMR (CDCl₃) δ (ppm): 3.15-3.23 (4H, m), 3.83-3.97 (4H, m), 7.01-7.10 (2H, m), 7.48-7.60 (2H, m).

### REFERENCE EXAMPLE 13

2-(4-Ethoxycarbonylpiperidino)benzonitrile

By using the same method as described in Reference Example 12, the subject compound is synthesized from 2-fluorobenzonitrile and ethyl isonipecotate.

¹H-NMR (CDCl₃) δ (ppm): 1.28 (3H, t, J=7.1 Hz), 1.92-2.10 (4H, m), 2.41-2.51 (1H, m), 2.84-2.94 (2H, m), 3.51-3.58 (2H, m), 4.17 (2H, q, J=7.1 Hz), 6.96-7.02 (2H, m), 7.44-7.56 (2H, m).

### REFERENCE EXAMPLE 14

2-Piperidinobenzonitrile

By using the same method as described in Reference Example 12, the subject compound is synthesized from 2-fluorobenzonitrile and piperidine.

¹H-NMR (CDCl₃) δ (ppm): 1.54-1.62 (2H, m), 1.70-1.79 (4H, m), 3.11-3.16 (4H, m), 6.90-6.99 (2H, m), 7.40-7.51 (2H, m).

### REFERENCE EXAMPLE 15

2-(4-Methyl-1-piperazinyl)benzonitrile

By using the same method as described in Reference Example 12, the subject compound is synthesized from 2-fluorobenzonitrile and 1-methylpiperazine.

¹H-NMR (CDCl₃) δ (ppm): 2.36 (3H, s), 2.60-2.65 (4H, m), 3.21-3.26 (4H, m), 6.97-7.03 (2H, m), 7.45-7.56 (2H, m).

### REFERENCE EXAMPLE 16

2-Thiomorpholinobenzonitrile

By using the same method as described in Reference Example 12, the subject compound is synthesized from 2-fluorobenzonitrile and thiomorpholine.

¹H-NMR (CDCl₃) δ (ppm): 2.84-2.88 (4H, m), 3.42-3.47 (4H, m), 7.00-7.06 (2H, m), 7.55 (1H, dd, J=6.9 Hz), 7.58 (1H, d, J=6.9 Hz).

### REFERENCE EXAMPLE 17

3-Morpholinobenzonitrile

3-Fluorobenzonitrile (4.35 g, 36.0 millimole) is dissolved with acetonitrile (40 ml), morpholine (110 ml, 1.25 mole) is added, and the solution is stirred for 3 days and nights at 110 degrees Celsius. The reaction solution is concentrated, the obtained oily material is purified using silica gel chromatography (eluted with chloroform), and the subject compound (1.80 g, 27%) is obtained as oily material.

¹H-NMR (CDCl₃) δ (ppm): 3.15-3.19 (4H, m), 3.83-3.88 (4H, m), 7.09-7.18 (3H, m), 7.34 (1H, dd, J=7.6 Hz, 7.6 Hz).

### REFERENCE EXAMPLE 18

3-Morpholinobenzonitrile

3-Cyanophenol (1.77 g, 14.9 millimole) and triethylamine (6.30 ml, 45.2 millimole) were dissolved with methylene chloride (75 ml), and the solution is stirred under an argon gas environment at −10 degrees Celsius. The solution of trifluoroacetic andydride (3.79 ml, 22.5 millimole), dissolved with methylene chloride (15 ml), is dripped, and the mixture is stirred for 1.5 hours. After completion of the reaction, the solvent is removed under reduced pressure. A sodium hydrogencarbonate aqueous solution is added to the obtained concentrated residuum, and extraction is performed with methylene chloride. The organic layer is washed with a saturated saline solution, and dried using sodium sulfate. After the desiccant is separated through filtration, the filtrate is concentrated. The obtained concentrated residuum is purified using silica gel column chromatography (chloroform/methanol=100), and oily material is obtained. The obtained oily material is dissolved with acetonitrile (40 ml). Morpholine (33.0 ml, 378 millimole) is added to the solution, and the solution is stirred for 3 days while the solvent is refluxed. The solvent and the reagent which did not react were removed under reduced pressure. Water is added to the obtained concentrated residuum, and extraction is performed using chloroform. After the organic layer is dried (sodium sulfate), the desiccant is separated through filtration, and the filtrate is concentrated. The obtained concentrated residuum is purified using silica gel column chromatography (eluted with chloroform/hexane=1/2), and the subject compound is obtained as oily material (0.74 g, 26%).

¹H-NMR (CDCl₃) δ (ppm): 3.15-3.19 (4H, m), 3.83-3.88 (4H, m), 7.09-7.18 (3H, m), 7.34 (1H, dd, J=7.6 Hz, 7.6 Hz).

### REFERENCE EXAMPLE 19

3-Piperidinobenzonitrile

By using the same method as described in Reference Example 17, the subject compound is synthesized from 3-fluorobenzonitrile and piperidine.

¹H-NMR (CDCl₃) δ (ppm): 1.55-1.73 (6H, m), 3.17-3.23 (4H, m), 7.03 (1H, d, J=7.6 Hz), 7.08-7.12 (2H, m), 7.24-7.29 (1H, m).

### REFERENCE EXAMPLE 20

3-(4-Methyl-1-piperazinyl)benzonitrile

By using the same method as described in Reference Example 17, the subject compound is synthesized from 3-fluorobenzonitrile and 1-methylpiperazine.

¹H-NMR (CDCl₃) δ (ppm): 2.35 (3H, s), 2.55-2.60 (4H, m), 3.21-3.25 (4H, m), 7.06-7.13 (3H, m), 7.28-7.31 (1H, m).

### REFERENCE EXAMPLE 21

4-Morpholinobenzonitrile

By using the same method as described in Reference Example 12, the subject compound is synthesized from 4-fluorobenzonitrile and morpholine.

¹H-NMR (CDCl₃) δ (ppm): 3.26-3.30 (4H, m), 3.83-3.87 (4H, m), 6.86 (2H, d, J=8.9 Hz), 7.51 (2H, d, J=8.9 Hz).

### REFERENCE EXAMPLE 22

4-Piperidinobenzonitrile

By using the same method as described in Reference Example 12, the subject compound is synthesized from 4-fluorobenzonitrile and piperidine.

¹H-NMR (CDCl₃) δ (ppm): 1.60-1.75 (6H, m), 3.30-3.38 (4H, m), 6.83 (2H, d, J=8.9 Hz), 7.45 (2H, d, J=8.9 Hz).

### REFERENCE EXAMPLE 23

4-(4-Methyl-1-piperazinyl)benzonitrile

By using the same method as described in Reference Example 12, the subject compound is synthesized from 4-fluorobenzonitrile and 1-methylpiperazine.

¹H-NMR (CDCl₃) δ (ppm): 2.35 (3H, s), 2.52-2.59 (4H, m), 3.31-3.39 (4H, m), 6.86 (2H, d, J=8.9 Hz), 7.49 (2H, d, J=8.9 Hz).

### REFERENCE EXAMPLE 24

4-(1-Pyrrolidinyl)benzonitrile

By using the same method as described in Reference Example 12, the subject compound is synthesized from 4-fluorobenzonitrile and pyrrolidine.

¹H-NMR (CDCl₃) δ (ppm): 2.01-2.09 (4H, m), 3.29-3.34 (4H, m), 6.49 (2H, d, J=8.9 Hz), 7.43 (2H, d, J=8.9 Hz).

### REFERENCE EXAMPLE 25

4-Thiomorpholinobenzonitrile

By using the same method as described in Reference Example 12, the subject compound is synthesized from 4-fluorobenzonitrile and thiomorpholine.

¹H-NMR (CDCl₃) δ (ppm): 2.62-2.75 (4H, m), 3.74-3.80 (4H, m), 6.81 (2H, d, J=8.9 Hz), 7.49 (2H, d, J=8.9 Hz).

### REFERENCE EXAMPLE 26

7-Ethylamino-6-nitro-4(3H)-quinazoline (the compound described in WO95/06649)

7-Chloro-6-nitro-4(3H)-quinazoline (20.0 g, 88.7 millimole) is suspended into n-butanol (150 ml), a 70%-ethylamine aqueous solution (120 ml) is added, and the solution is stirred for 15 minutes at room temperature. After the solution is made uniform, (the solution is placed in a closed tube and is heated for 9 hours in an oil bath (bathing oil temperature set to 100 degrees Celsius). After completion of the reaction, the solution is left to cool down, and precipitated yellow solid (primary crystal) is separated through filtration. After the primary crystal is washed with methanol and ether, it is dried, and the target subject compound is obtained (8.27 g, 40%). Moreover, the filtrate during the process of obtaining primary crystal is concentrated, and the crystal, which is precipitated, is separated through filtration. Then the same above process is carried out, and the target subject compound of the secondary crystal is obtained (5.84 g, 24%).

### REFERENCE EXAMPLE 27

4-Chloro-7-ethylamino-6-nitroquinazoline

7-Ethylamino-6-nitro-4(3H)-quinazoline (30.0 g, 128 millimole) is suspended in phosphorus oxychloride (270 ml, 2.90 mole), and the solution is heated for 2 hours at 110 degrees Celsius under an argon gas environment. (The solution became uniform.) After dissipation of the source materials for the reaction is confirmed, the phosphorus oxychloride, which did not react, is removed under reduced pressure. After the solution is azeotropically boiled with toluene, the obtained oily material is dissolved using a minimum necessary amount of tetrahydrofuran. The above obtained tetrahydrofuran solution is poured into ice water in which a sufficient amount of sodium hydrogencarbonate is added, and extraction is performed with ethyl acetate. After the organic layer is dried with the desiccant (magnesium sulfate anhydride), the desiccant is separated through filtration. The filtrate is concentrated under reduced pressure, and the subject compound (33.4 g) is obtained.

¹H-NMR (CDCl₃) δ (ppm): 1.45 (3H, t, J=7.3 Hz), 3.42-3.47 (2H, m), 7.18 (1H, s), 7.79 (1H, br), 8.84 (1H, s), 9.11 (1H, s).

### REFERENCE EXAMPLE 28

7-Ethylamino-4-(2-methylaminobenzylamino)-6-nitroquinazoline

A solution, in which aluminum lithium hydride (3.28 g, 86.4 millimole) is suspended in tetrahydrofuran (100 ml), is stirred under ice-chilled conditions under an argon gas environment. The solution in which the 2-methylaminobenzonitrile (3.80 g, 28.8 millimole) obtained in Reference Example 1 is dissolved with tetrahydrofuran (30 ml) is added dropwise. After the dripping process is completed, the solution is stirred for 3 hours while the solvent is refluxed. After completion of the reaction, the reaction solution is cooled and sodium sulfate decahydrate is added slowly until foaming stops. Thereafter, the material which is not dissolved is separated through filtration, the filtrate is concentrated under reduced pressure, and oily 2-methylaminobenzylamine is obtained.

The obtained oily material and triethylamine (20.0 ml, 143 millimole) were dissolved using tetrahydrofuran (100 ml), 4-chloro-7-ethylamino-6-nitroquinazoline (the compound obtained in Reference Example 27, 6.47 g, 25.6 millimole) is added, and the solution is stirred for one night at room temperature. After completion of the reaction, the solvent is removed under reduced pressure, water is added to the obtained residuum, and precipitated solid is separated through filtration. Furthermore, the solution is washed with ether-methanol, and the subject compound (6.88 g, 76%) is obtained.

¹H-NMR (DMSO-d₆) δ (ppm): 1.30 (3H, t, J=7.2 Hz), 2.76 (3H, d, J=4.5 Hz), 3.36-3.43 (2H, m), 4.60 (2H, d, J=5.4 Hz), 5.73 (1H, q, J=4.5 Hz), 6.50-6.59 (2H, m), 6.85 (1H, s), 7.08 -7.16 (2H, m), 7.74 (1H, t, J=5.4 Hz), 8.36 (1H, s), 9.05 (1H, t, J=5.7 Hz), 9.26 (1H, s).

### REFERENCE EXAMPLE 29

7-Ethylamino-4-(4-methylaminobenzylamino)-6-nitroquinazoline The compound obtained in Reference Example 2 is reduced with aluminum lithium hydride. Thereafter, by using the same method as described in Reference Example 28, the subject compound is obtained from the compound obtained hereinabove and 4-chloro-7-ethylamino-6-nitroquinazoline.

¹H-NMR (DMSO-d₆) δ (ppm): 1.31 (3H, t, J=7.1 Hz), 2.84 (3H, d, J=5.3 Hz), 3.35 (2H, q, J=7.1 Hz), 4.62 (2H, d, J=5.3 Hz), 5.45-5.60 (1H, br), 6.64 (2H, d, J=8.6 Hz), 6.83 (1H, s), 7.19 (2H, d, J=8.6 Hz), 7.71 (1H, t, J=5.3 Hz), 8.32 (1H, s), 9.06 (1H, t, J=5.3 Hz), 9.28 (1H, s).

### REFERENCE EXAMPLE 30

4-(4-Benzylaminobenzylamino)-7-ethylamino-6-nitroquinazoline

The compound obtained in Reference Example 3 is reduced with aluminum lithium hydride. Thereafter, by using the same method as described in Reference Example 28, the subject compound is obtained from the compound obtained hereinabove and 4-chloro-7-ethylamino-6-nitroquinazoline.

¹H-NMR (DMSO-d₆) δ (ppm): 1.27 (3H, t, J=7.2 Hz), 3.33-3.41 (2H, m), 4.24 (2H, d, J=4.5 Hz), 4.56 (2H, d, J=5.4 Hz), 6.20 (1H, br), 6.53 (2H, d, J=8.4 Hz), 6.83 (1H, s), 7.06 (2H, d, J=8.4 Hz), 7.16-7.35 (5H, m), 7.71 (1H, t, J=5.2 Hz), 8.32 (1H, s), 9.03 (1H, t, J=5.4 Hz), 9.25 (1H, s).

### REFERENCE EXAMPLE 31

7-Ethylamino-4-(4-isopropylaminobenzylamino)-6-nitroquinazoline

The compound obtained in Reference Example 4 is reduced with aluminum lithium hydride. Thereafter, by using the same method as described in Reference Example 28, the subject compound is obtained from the compound obtained hereinabove and 4-chloro-7-ethylamino-6-nitroquinazoline.

¹H-NMR (CDCl₃) δ (ppm): 1.22 (6H, d, J=6.3 Hz), 1.40 (3H, t, J=7.3 Hz), 3.32-3.42 (2H, m), 3.58-3.66 (1H, m), 4.68 (2H, d, J=5.0 Hz), 6.03 (1H, br), 6.58 (2H, d, J=8.6 Hz), 6.98 (1H, s), 7.20 (2H, d, J=8.6 Hz), 7.68 (1H, t, J=4.6 Hz), 8.54 (1H, s), 8.66 (1H, s), 9.40 (1H, br).

### REFERENCE EXAMPLE 32

7-Ethylamino-6-nitro-4-(4-propylaminobenzylamino)quinazoline

The compound obtained in Reference Example 5 is reduced with aluminum lithium hydride. Thereafter, by using the same method as described in Reference Example 28, the subject compound is obtained from the compound obtained hereinabove and 4-chloro-7-ethylamino-6-nitroquinazoline.

¹H-NMR (DMSO-d₆) δ (ppm): 0.91 (3H, t, J=7.4 Hz), 1.27 (3H, t, J=7.2 Hz), 1.50-1.60 (2H, m), 2.90-2.96 (2H, m), 3.32-3.40 (2H, m), 4.57 (2H, d, J=5.9 Hz), 5.48 (1H, br), 6.51 (2H, d, J=8.4 Hz), 6.85 (1H, s), 7.09 (2H, d, J=8.4 Hz), 7.72 (1H, t, J=5.9 Hz), 8.33 (1H, s), 9.07 (1H, br), 9.27 (1H, s).

### REFERENCE EXAMPLE 33

7-Ethylamino-4-(4-ethylaminobenzylamino)-6-nitroquinazoline

The compound obtained in Reference Example 6 is reduced with aluminum lithium hydride. Thereafter, by using the same method as described in Reference Example 28, the subject compound is obtained from the compound obtained hereinabove and 4-chloro-7-ethylamino-6-nitroquinazoline.

¹H NMR (DMSO-d₆) δ (ppm): 1.14 (3H, t, J=7.3 Hz), 1.28 (3H, t, J=7.1 Hz), 3.00 (2H, q, J=7.3 Hz), 3.38 (2H, q, J=7.1 Hz), 4.58 (2H, d, J=5.6 Hz), 5.40 (1H, br), 6.50 (2H, d, J=8.6 Hz), 6.83 (1H, s), 7.09 (2H, d, J=8.6 Hz), 7.74 (1H, t, J=5.6 Hz), 8.33 (1H, s), 9.09 (1H, t, J=5.6 Hz, 9.28 (1H, s).

### REFERENCE EXAMPLE 34

7-Ethylamino-4-[2-(2-morpholinoethylamino)benzylamino]-6-nitroquinazoline

The compound obtained in Reference Example 7 is reduced with aluminum lithium hydride. Thereafter, by using the same method as described in Reference Example 28, the subject compound is obtained from the compound obtained hereinabove and 4-chloro-7-ethylamino-6-nitroquinazoline.

¹H-NMR (CDCl₃) δ (ppm): 1.40 (3H, t, J=7.2 Hz), 2.37-2.55 (4H, m), 2.58 (2H, t, J=6.2 Hz), 3.20-3.25 (2H, m), 3.31-3.41 (2H, m), 3.44-3.53 (4H, m), 4.80 (2H, d, J=5.5 Hz), 5.12 (1H, br), 6.32 (1H, t, J=5.2 Hz), 6.65-6.74 (2H, m), 6.98 (1H, s), 7.21-7.29 (2H, m), 7.68 (1H, t, J=4.7 Hz), 8.55 (1H, s), 8.70 (1H, s), 7.21-7.29 (2H, m), 7.68 (1H, t, J=4.7 Hz), 8.55 (1H, s), 8.70 (1H, s).

### REFERENCE EXAMPLE 35

7-Ethylamino-4-[2-(3-

morpholinopropylamino)benzylamino]-6-nitroquinazoline

The compound obtained in Reference Example 8 is reduced with aluminum lithium hydride. Thereafter, by using the same method as described in Reference Example 28, the subject compound is obtained from the compound obtained hereinabove and 4-chloro-7-ethylamino-6-nitroquinazoline.

¹H-NMR (CDCl₃) δ (ppm): 1.40 (3H, t, J=7.2 Hz), 1.72-1.83 (2H, m), 2.32-2.42 (6H, m), 3.17 (2H, t, J=6.7 Hz), 3.34-3.42 (2H, m), 3.64-3.71 (4H, m), 4.82 (2 h, d, J=5.9 Hz), 5.30 (1H, br), 6.32 (1H, br), 6.67 (1H, dd, J=7.4 Hz, 7.9 Hz), 6.71 (1H, d, J=7.4 Hz), 6.98 (1H, s), 7.20-7.23 (2H, m), 7.70 (1H, t, J=4.5 Hz), 8.54 (1H, s), 8.68 (1H, s).

### REFERENCE EXAMPLE 36

7-Ethylamino-4-[2-(2-hydroxyethylamino)benzylamino]-6-nitroquinazoline

The compound obtained in Reference Example 9 is reduced with aluminum lithium hydride. Thereafter, by using the same method as described in Reference Example 28, the subject compound is obtained from the compound obtained hereinabove and 4-chloro-7-ethylamino-6-nitroquinazoline.

¹H-NMR (DMSO-d₆) δ (ppm): 1.26 (3H, t, J=7.1 Hz), 3.09-3.16 (2H, m), 3.58 (2H, q, J=7.1 Hz), 3.54-3.62 (2H, m), 4.61 (2H, d, J=5.3 Hz), 4.72 (1H, t, J=5.3 Hz), 5.74 (1H, t, J=5.3 Hz), 6.55-6.60 (2H, m), 6.86 (1H, s), 7.07-7.15 (2H, m), 7.77 (1H, t, J=5.3 Hz), 8.38 (1H, s), 9.09 (1H, t, J=5.6 Hz), 9.25 (1H, s).

### REFERENCE EXAMPLE 37

7-Ethylamino-4-[4-(2-hydroxyethylamino)benzylamino]-6-nitroquinazoline

The compound obtained in Reference Example 10 is reduced with aluminum lithium hydride. Thereafter, by using the same method as described in Reference Example 28, the subject compound is obtained from the compound obtained hereinabove and 4-chloro-7-ethylamino-6-nitroquinazoline.

¹H-NMR (DMSO-d₆) δ (ppm): 1.26 (3H, t, J=6.9 Hz), 3.03-3.09 (2H, m), 3.38 (2H, q, J=6.9 Hz), 3.50-3.56 (2H, m), 4.58 (2H, d, J=5.0 Hz), 4.65 (1H, t, J=5.6 Hz), 5.42 (1H, t, J=5.0 Hz), 6.54 (2H, d, J=6.6 Hz), 6.85 (1H, s), 7.10 (2H, d, J=6.6 Hz), 7.71 (1H, br), 8.34 (1h, s), 9.08 (1H, br), 9.27 (1H, s).

### REFERENCE EXAMPLE 38

7-Ethylamino-4-[2-(2-methoxyethylamino)benzylamino]-6-nitroquinazoline

The compound obtained in Reference Example 11 is reduced with aluminum lithium hydride. Thereafter, by using the same method as described in Reference Example 28, the subject compound is obtained from the compound obtained hereinabove and 4-chloro-7-ethylamino-6-nitroquinazoline.

¹H-NMR (DMSO-d₆) δ (ppm): 1.29 (3H, t, J=6.9 Hz), 3.18-3.23 (5H, m), 3.39 (2H, q, J=6.9 Hz), 3.49 (2H, t, J=5.4 Hz), 4.64 (2H, d, J=5.9 Hz), 5.73 (1H, br), 6.58 (1H, d, J=7.9 Hz), 6.60 (1H, d, J=6.9 Hz), 6.86 (1H, s), 7.07-7.18 (2H, m), 7.76 (1H, t, J=5.3 Hz), 8.37 (1H, s), 9.07 (1H, br), 9.25 (1H, s).

### REFERENCE EXAMPLE 39

7-Ethylamino-4-(2-morpholinobenzylamino)-6-nitroquinazoline

The solution in which aluminum lithium hydride (6.36 g, 168 millimole) is suspended with dried tetrahydrofuran (200 ml) is stirred under ice-chilled conditions under an argon gas environment. The solution, in which 2-morpholinobenzonitrile (9.56 g, 50.8 millimole), which is obtained in Reference Example 12, is dissolved with tetrahydrofuran (150 ml), and dripped into the above solution over a time period of 30 minutes. After completion of dripping, the reaction solution is stirred for 2 hours while the solvent is refluxed. After completion of the reaction, the reaction solution is cooled and sodium sulfate decahydrate is added slowly until foaming stopped. Thereafter, the material which is not dissolved is separated through filtration, the filtrate is concentrated under reduced pressure, and oily 2-morpholinobenzylamine is obtained.

The obtained oily material and triethylamine (35.4 ml, 253 millimole) were dissolved with tetrahydrofuran (200 ml), 4-chloro-7-ethylamino-6-nitroquinazoline (7.34 g, 30.6 millimole) is added, and the solution is stirred for one night at room temperature. After completion of the reaction, the solvent is removed under reduced pressure, the obtained residuum is purified using silica gel chromatography (eluted with chloroform/methanol=100), and the subject compound (7.17 g, 60%) is obtained.

¹H-NMR (CDCl₃) δ (ppm): 1.41 (3H, t, J=7.3 Hz), 3.04-3.08 (4H, m), 3.38 (2H, q, J=7.3 Hz), 3.90-3.94 (4H, m), 4.97 (2H, d, J=4.6 Hz), 7.03 (1H, s), 7.12-7.18 (1H, m), 7.25 (1H, d, J=7.9 Hz), 7.31-7.40 (2H, m), 7.74 (1H, br), 7.87 (1H, s), 8.54 (1H, s), 8.72 (1H, s).

### REFERENCE EXAMPLE 40

7-Ethylamino-4-[2-(4-hydroxymethylpiperidino)benzylamino]-6-nitroquinazoline

The compound obtained in Reference Example 13 is reduced with aluminum lithium hydride. Thereafter, by using the same method as described in Reference Example 39, the subject compound is obtained from the compound obtained hereinabove and 4-chloro-7-ethylamino-6-nitroquinazoline.

¹H-NMR (DMSO-d₆) δ (ppm): 1.30 (3H, t, J=7.3 Hz), 1.32-1.50 (2H, m), 1.74-1.79 (3H, m), 2.62-2.70 (2H, m), 3.07-3.12 (2H, m), 3.30-3.50 (4H, m), 4.45 (1H, t, j=5.3 Hz), 4.84 (2H, J=5.3 Hz), 6.87 (1H, s), 6.99 (1H, dd, J=6.6, 6.9 Hz), 7.13 (1H, d, J=7.3 Hz), 7.19-7.24 (2H, m), 7.74 1H, t, 5.3 Hz), 8.31 (1H, s), 9.06 (1H, t, J=5.6 Hz), 9.31 (1H, s).

### REFERENCE EXAMPLE 41

7-Ethylamino-6-nitro-4-(2-piperidinobenzylamino)quinazoline

The compound obtained in Reference Example 14 is reduced with aluminum lithium hydride. Thereafter, by using the same method as described in Reference Example 39, the subject compound is obtained from the compound obtained hereinabove and 4-chloro-7-ethylamino-6-nitroquinazoline.

¹H-NMR (CDCl₃) δ (ppm): 1.41 (3H, t, J=7.3 Hz), 1.61-1.79 (2H, m), 1.81-1.88 (4H, m), 2.95-3.04 (4H, m), 3.38 (2H, q, J=7.3 Hz), 4.95 (2H, d, J=5.0 Hz), 6.98 (1H, s), 7.10 (1H, dd, J=7.3 Hz, 8.9 Hz), 7.22-7.36 (3H, m), 7.71 (1H, t, J=5.0 Hz), 8.37 (1H, br), 8.53 (1H, s), 8.72 (1H, s).

### REFERENCE EXAMPLE 42

7-Ethylamino-4-[2-(4-methyl-1-piperazinyl)benzylamino]-6-nitroquinazoline

The compound obtained in Reference Example 15 is reduced with aluminum lithium hydride. Thereafter, by using the same method as described in Reference Example 39, the subject compound is obtained from the compound obtained hereinabove and 4-chloro-7-ethylamino-6-nitroquinazoline.

¹H-NMR (CDCl₃) δ (ppm): 1.41 (3H, t, J=7.3 Hz), 2.42 (3H, s), 2.70-2.80 (4H, m), 3.05-3.15 (4H, m), 3.40 (2H, q, J=7.3 Hz), 3.70 (1H, br), 4.95 (2H, s), 6.94 (1H, s), 7.12 (1H, dd, J=7.3 Hz, 7.3 Hz), 7.23-7.36 (3H, m), 7.73 (1H, br), 8.44 (1H, br), 8.89 (1H, s).

### REFERENCE EXAMPLE 43

7-Ethylamino-6-nitro-4-(2-thiomorpholinobenzylamino)quinazoline

The compound obtained in Reference Example 16 is reduced with aluminum lithium hydride. Thereafter, by using the same method as described in Reference Example 39, the subject compound is obtained from the compound obtained hereinabove and 4-chloro-7-ethylamino-6-nitroquinazoline.

¹H-NMR (DMSO-d₆) δ (ppm): 1.31 (3H, t, J=7.1 Hz), 2.70-2.80 (4H, m), 3.10-3.18 (4H, m), 3.35-3.44 (2H, m), 4.83 (2H, d, J=5.3 Hz), 6.86 (1H, s), 7.04 (1H, dd, J=7.3 Hz, 7.6 Hz), 7.15 (1H, d, J=7.9 Hz), 7.21-7.27 (2H, m), 7.73 (1H, t, J=5.3 Hz), 8.32 (1H, s), 9.03 (1H, t, J=5.4 Hz), 9.32 (1H, s).

### REFERENCE EXAMPLE 44

7-Ethylamino-4-(3-morpholinobenzylamino)-6-nitroquinazoline

The compound obtained in Reference Example 17 or Reference Example 18 is reduced with aluminum lithium hydride. Thereafter, by using the same method as described in Reference Example 39, the subject compound is obtained from the compound obtained hereinabove and 4-chloro-7-ethylamino-6-nitroquinazoline.

¹H-NMR (DMSO-d₆) δ (ppm): 1.30 (3H, t, J=7.1 Hz), 3.06-3.15 (4H, m), 3.32-3.39 (2H, m), 3.71-3.78 (4H, m), 4.70 (2H, d, J=5.3 Hz), 6.79-6.85 (3H, m), 6.96 (1H, s), 7.17 (1H, dd, J=7.9 Hz), 7.72 (1H, t, J=5.3 Hz), 8.32 (1H, s), 9.13 (1H, t, J=5.6 Hz), 9.28 (1H, s).

### REFERENCE EXAMPLE 45

7-Ethylamino-6-nitro-4-(3-piperidinobenzylamino)quinazoline

The compound obtained in Reference Example 19 is reduced with aluminum lithium hydride. Thereafter, by using the same method as described in Reference Example 39, the subject compound is obtained from the compound obtained hereinabove and 4-chloro-7-ethylamino-6-nitroquinazoline.

¹H-NMR (CDCl₃) δ (ppm): 1.40 (3H, t, J=7.3 Hz), 1.53-1.73 (6H, m), 3.13-3.18 (4H, m), 3.32-3.40 (2H, m), 4.77 (2H, d, J=5.0 Hz), 6.29 (1H, br), 6.80-6.90 (2H, m), 6.96 (1H, s), 6.98 (1H, s), 7.17-7.27 (1H, m), 7.67 (1 h, t, J=4.6 Hz), 8.54 (1H, s), 8.71 (1H, s).

### REFERENCE EXAMPLE 46

7-Ethylamino-4-[3-(4-methyl-1-piperazinyl)benzylamino]-6-nitroquinazoline

The compound obtained in Reference Example 20 is reduced with aluminum lithium hydride. Thereafter, by using the same method as described in Reference Example 39, the subject compound is obtained from the compound obtained hereinabove and 4-chloro-7-ethylamino-6-nitroquinazoline.

¹H-NMR (CDCl₃) δ (ppm): 1.36 (3H, t, J=7.3 Hz), 2.31(3H, s), 2.49-2.58 (4H, m), 3.14-3.24 (4H, m), 3.29 (2H, q, J=7.3 Hz), 4.77 (2H, d, J=4.3 Hz), 6.73-6.93 (4H, m), 7.16-7.22 (2H, m), 7.61 (1H, t, J=4.3 Hz), 8.50 (1H, s), 8.82 (1H, s).

### REFERENCE EXAMPLE 47

7-Ethylamino-4-(4-morpholinobenzylamino)-6-nitroquinazoline

The compound obtained in Reference Example 21 is reduced with aluminum lithium hydride. Thereafter, by using the same method as described in Reference Example 39, the subject compound is obtained from the compound obtained hereinabove and 4-chloro-7-ethylamino-6-nitroquinazoline.

¹H-NMR (DMSO-d₆) δ (ppm): 1.26 (3H, t, J=7.1 Hz), 3.30-3.07 (4H, m), 3.34 (2H, q, J=7.1 Hz), 3.70-3.73 (4H, m), 4.64 (2H, d, J=5.3 Hz), 6.85 (1H, s), 6.90 (2H, d, J=8.6 Hz), 7.23 (2H, d, J=8.6 Hz), 7.74 (1H, br), 8.33 (1H, s), 9.15 (1H, br), 9.27 (1H, s).

### REFERENCE EXAMPLE 48

7-Ethylamino-6-nitro-4-(4-piperidinobenzylamino)quinazoline

The compound obtained in Reference Example 22 is reduced with aluminum lithium hydride. Thereafter, by using the same method as described in Reference Example 39, the subject compound is obtained from the compound obtained hereinabove and 4-chloro-7-ethylamino-6-nitroquinazoline.

¹H-NMR (CDCl₃) δ (ppm): 1.41 (3H, t, J=7.4 Hz), 1.50-1.80 (6H, m), 3.02-3.19 (4H, m), 3.33-3.43 (2H, m), 4.73 (2H, s), 6.20 (1H, br), 6.80-7.00 (2H, m), 7.21-7.30 (3H, m), 7.69 (1H, br), 8.54 (1H, s), 8.69 (1H, s).

### REFERENCE EXAMPLE 49

7-Ethylamino-4-[4-(4-methyl-1-piperazinyl)benzylamino]-6-nitroquinazoline

The compound obtained in Reference Example 23 is reduced with aluminum lithium hydride. Thereafter, by using the same method as described in Reference Example 39, the subject compound is obtained from the compound obtained hereinabove and 4-chloro-7-ethylamino-6-nitroquinazoline.

¹H-NMR (CDCl₃) δ (ppm) 1.39 (3H, t, J=7.3 Hz), 2.37 (3H, s), 2.55-2.62 (4H, m), 3.18-3.33 (4H, m), 3.38 (2H, q, J=7.3 Hz), 4.74 (2H, d, J=5.0 Hz), 6.15 (1H, br), 6.93 (2H, d, J=8.6 Hz), 6.99 (1H, s), 7.31 (2H, d, J=8.6 Hz), 7.69 (1H, br), 8.55 (1H, s), 8.69 (1H, s).

### REFERENCE EXAMPLE 50

7-Ethylamino-6-nitro-4-[4-(1-pyrrolidinyl)benzylamino]quinazoline

The compound obtained in Reference Example 24 is reduced with aluminum lithium hydride. Thereafter, by using the same method as described in Reference Example 39, the subject compound is obtained from the compound obtained hereinabove and 4-chloro-7-ethylamino-6-nitroquinazoline.

¹H-NMR (DMSO-d₆) δ (ppm): 1.30 (3H, t, J=7.1 Hz), 1.91-2.00 (4H, m), 3.17-3.22 (4H, m), 3.32-3.41(3H, m), 4.62 (2H, d, J=5.6 Hz), 6.48 (2H, d, J=8.6 Hz), 6.84 (1H, s), 7.19 (2H, d, J=8.6 Hz), 7.73 (1H, t, J=5.6 Hz), 8.27 (1H, s), 8.32 (1H, s).

### REFERENCE EXAMPLE 51

7-Ethylamino-6-nitro-4-(4-thiomorpholinobenzylamino)quinazoline

The compound obtained in Reference Example 25 is reduced with aluminum lithium hydride. Thereafter, by using the same method as described in Reference Example 39, the subject compound is obtained from the compound obtained hereinabove and 4-chloro-7-ethylamino-6-nitroquinazoline.

¹H-NMR (CDCl₃) δ (ppm): 1.41 (3H, t, J=7.2 Hz), 2.72-2.77 (4H, m), 3.36-3.43 (2H, m), 3.55-3.61 (4H, m), 4.74 (2H, d, J=4.9 Hz), 5.96 (1H, br), 6.90 (2H, d, J=8.9 Hz), 7.00 (1H, s), 7.30 (2H, d, J=8.9 Hz), 7.69 (1H, br), 8.56 (1H, s), 8.65 (1H, s).

### REFERENCE EXAMPLE 52

2-[N-(2-Hydroxyethyl)methylamino]benzonitrile

By using the same method as described in Reference Example 3, the subject compound is synthesized from 2-fluorobenzonitrile and N-methylethanolamine.

¹H-NMR (CDCl₃) δ (ppm): 2.60 (1H, br), 3.02 (3H, s), 3.49 (2H, t, J=5.8 Hz), 3.87 (2H, t, J=5.8 Hz), 6.89 (1H, dd, J=7.3 Hz, 7.6 Hz), 7.01 (1H, d, J=8.6 Hz), 7.39-7.52 (2H, m).

### REFERENCE EXAMPLE 53

7-Ethylamino-4-{2-[N-(2-hydroxyethyl)methylamino]benzylamino}-6-nitroquinazoline

The compound obtained in Reference Example 52 is reduced with aluminum lithium hydride. Thereafter, by using the same method as described in Reference Example 39, the subject compound is obtained from the compound obtained hereinabove and 4-chloro-7-ethylamino-6-nitroquinazoline.

¹H-NMR (CDCl₃) δ (ppm): 1.37 (3H, t, J=7.1 Hz), 1.95 (1H, br), 2.75 (3H, s), 3.19 (2H, t, J=5.0 Hz), 3.27-3.38 (2H, m), 3.85 (2H, t, J=5.0 Hz), 4.95 (2H, d, J=5.3 Hz), 6.88 (1H, d), 7.14 (1H, dd, J=7.3 Hz, 7.3 Hz), 7.23-7.32 (2H, m), 7.46 (1H, d, J=7.3 Hz), 7.63 (1H, br), 8.20 (1H, br), 8.45 (1H, s), 8.96 (1H, s).

### REFERENCE EXAMPLE 54

4-(3-Hydroxymethylpiperidino)benzonitrile

By using the same method as described in Reference Example 3, the subject compound is synthesized from 4-fluorobenzonitrile and 3-hydroxymethylpiperidine.

¹H-NMR (CDCl₃) δ (ppm): 1.20-1.32 (1H, m), 1.56-1.73 (1H, m), 1.74-1.95 (4H, m), 2.72-2.80 (1H, m), 2.87-2.98 (1H, m), 3.47-3.76 (3H, m), 3.86-3.92 (1H, m), 6.87 (2H, d, J=8.9 Hz), 7.44 (2H, d, J=8.9 Hz).

### REFERENCE EXAMPLE 55

7-Ethylamino-4-[4-(3-hydroxymethylpiperidino)benzylamino]-6-nitroquinazoline

The compound obtained in Reference Example 54 is reduced with aluminum lithium hydride. Thereafter, by using the same method as described in Reference Example 39, the subject compound is obtained from the compound obtained hereinabove and 4-chloro-7-ethylamino-6-nitroquinazoline.

¹H-NMR (DMSO-d₆) δ (ppm): 1.00-1.15 (1H, m), 1.30 (3H, t, J=7.2 Hz), 1.36-1.72 (4H, m), 2.34-2.39 (1H, m), 2.57-2.66 (1H, m), 3.18-3.40 (4H, m), 3.50-3.56 (1H, m), 3.62-3.66 (1H, m), 4.45-4.49 (1H, m), 4.64 (2H, d, J=5.4 Hz), 6.85 (1H, s), 6.86 (2H, d, J=8.9 Hz), 7.21 (2H, d, J=8.9 Hz), 7.71 (1H, br), 8.32 (1H, s), 9.09 (1H, t, J=5.4 Hz), 9.27 (1H, s).

### REFERENCE EXAMPLE 56

2-(1-Imidazolyl)benzonitrile

2-Fluorobenzonitrile (0.70 g, 5.78 millimole) is dissolved with acetonitrile (1 ml), imidazole sodium salt (1.34 g, 148 millimole) is added, and the solution is stirred for 1.5 hours at 100 degrees Celsius. The reaction solution is concentrated under reduced pressure, the obtained residuum is purified using silica gel chromatography (eluted with chloroform/hexane=1/2, then eluted with chloroform at the end by gradually increasing the contents of the chloroform.), and the subject compound (0.96 g, 98%) is obtained.

¹H-NMR (CDCl₃) δ (ppm): 7.26 (1H, s), 7.37 (1H, s), 7.41-7.57 (2H, m), 7.72-7.87 (3H, m).

### REFERENCE EXAMPLE 57

7-Ethylamino-4-[2-(1-imidazolyl)benzylamino]-6-nitroquinazoline

The compound obtained in Reference Example 56 is reduced with aluminum lithium hydride. Thereafter, by using the same method as described in Reference Example 39, the subject compound is obtained from the compound obtained hereinabove through the reduction and 4-chloro-7-ethylamino-6-nitroquinazoline.

¹H-NMR (DMSO-d₆) δ (ppm): 1.28 (3H, t, J=7.1 Hz), 3.35-3.40 (2H, m), 4.56 (2H, d, J=5.3 Hz), 6.86 (1H, s), 7.09 (1H, s), 7.34-7.55 (5H, m), 7.76 (1H, t, J=5.3 Hz), 7.92 (1H, s), 8.27 (1H, s), 9.13 (1H, t, J=5.2 Hz), 9.28 (1H, s).

### REFERENCE EXAMPLE 58

2-(1-Perhydroazocinyl)benzonitrile

By using the same method as described in Reference Example 12, the subject compound is obtained from 2-fluorobenzonitrile and heptamethyleneimine.

¹H-NMR (CDCl₃) δ (ppm): 1.55-1.65 (6H, m), 1.76-1.82 (4H, m), 3.66-3.71 (4H, m), 6.64 (1H, dd, J=6.9 Hz, 7.9 Hz), 6.80 (1H, d, J=8.6 Hz), 7.25-7.34 (1H, m), 7.43 (1H, d, J=7.9 Hz).

### REFERENCE EXAMPLE 59

4-[2-(1-Perhydroazocinyl)benzylamino]-7-ethylamino-6-nitroquinazoline

The compound obtained in Reference Example 58 is reduced with aluminum lithium hydride. Thereafter, by using the same method as described in Reference Example 39, the subject compound is obtained from the compound obtained hereinabove through the reduction and 4-chloro-7-ethylamino-6-nitroquinazoline.

¹H-NMR (CDCl₃) δ (ppm): 1.36 (3H, t, J=7.1 Hz), 1.68-1.72 (1H, m), 3.14-3.15 (4H, m), 3.27-3.32 (2H, m), 4.96 (2H, d, J=5.3 Hz), 6.92 (1H, s), 7.00-7.06 (1H, m), 7.24-7.33 (3H, m), 7.48 (1 h, br), 7.64 (1 h, br), 8.48 (1H, s), 8.77 (1H, s).

### REFERENCE EXAMPLE 60

2-Propylaminobenzonitrile

By using the same method as described in Reference Example 3, the subject compound is synthesized from 2-fluorobenzonitrile and n-propylamine.

¹H-NMR (CDCl₃) δ (ppm): 1.00 (3H, t, J=7.3 Hz), 1.58-1.72 (2H, m), 3.10-3.15 (2H, m), 4.55 (1H, br), 6.58-6.65 (2H, m), 7.32-7.38 (2H, m).

### REFERENCE EXAMPLE 61

7-Ethylamino-6-nitro-4-(2-propylaminobenzylamino)quinazoline

The compound obtained in Reference Example 60 is reduced with aluminum lithium hydride. Thereafter, by using the same method as described in Reference Example 28, the subject compound is obtained from the compound obtained hereinabove through the reduction and 4-chloro-7-ethylamino-6-nitroquinazoline.

¹H-NMR (DMSO-d₆) δ (ppm): 0.88 (3H, t, J=7.3 Hz), 1.28 (3H, t, J=7.1 Hz), 1.51-1.59 (2H, m), 2.98-3.02 (2H, m), 3.33-3.41 (2H, m), 4.64 (2H, d, J=5.6 Hz), 5.61 (1H, br), 6.51 6.58 (2H, m), 6.86 (1H, s), 7.06-7.15 (2H, m), 7.78 (1H, t, J=5.3 Hz), 8.35 (1H, s), 9.10 (1H, t, J=5.6 Hz), 9.25 (1H, s).

### REFERENCE EXAMPLE 62

2-Isopropylaminobenzonitrile

By using the same method as described in Reference Example 3, the subject compound is synthesized from 2-fluorobenzonitrile and isopropylamine.

¹H-NMR (CDCl₃) δ (ppm): 1.23 (6H, d, J=6.3 Hz), 3.61-3.75 (1H, m), 4.40 (1H, br), 6.57-6.66 (2H, m), 7.30-7.37 (2H, m).

### REFERENCE EXAMPLE 63

7-Ethylamino-4-(2-isopropylaminobenzylamino)-6-nitroquinazoline

The compound obtained in Reference Example 62 is reduced with aluminum lithium hydride. Thereafter, by using the same method as described in Reference Example 28, the subject compound is obtained from the compound obtained hereinabove through the reduction and 4-chloro-7-ethylamino-6-nitroquinazoline.

¹H-NMR (DMSO-d₆) δ (ppm): 1.08 (6H, d, J=6.3 Hz), 1.28 (3H, t, J=7.3 Hz), 3.34-3.40 (2H, m), 3.43-3.59 (1H, m), 4.64 (2H, d, J=5.6 Hz), 5.51 (1H, br), 6.51-6.58 (2H, m), 6.86 (1H, s), 7.06-7.17 (2H, m), 7.77 (1H, t, J=5.3 Hz), 8.37 (1H, s), 9.07 (1H, t, J=5.6 Hz), 9.24 (1H, s).

### REFERENCE EXAMPLE 64

2-(3-Hydroxymethylpiperidino)benzonitrile

By using the same method as described in Reference Example 12, the subject compound is synthesized from 2-fluorobenzonitrile and 3-(hydroxymethyl)piperidine.

¹H-NMR (CDCl₃) δ (ppm): 1.23-1.33 (1H, m), 1.72-1.85 (3H, m), 1.79-2.17 (2H, m), 2.80-2.94 (2H, m), 3.35-3.52 (2H, m), 3.59-3.74 (2H, m), 6.90-7.05 (2H, m), 7.43-7.56 (2H, m).

### REFERENCE EXAMPLE 65

7-Ethylamino-4-[2-(3-hydroxymethylpiperidino)benzylamino]-6-nitroquinazoline

The compound obtained in Reference Example 64 is reduced with aluminum lithium hydride. Thereafter, by using the same method as described in Reference Example 39, the subject compound is obtained from the compound obtained hereinabove through the reduction and 4-chloro-7-ethylamino-6-nitroquinazoline.

¹H-NMR (DMSO-d₆) δ (ppm): 1.07-1.02 (1H, m), 1.30 (3H, t, J=7.1 Hz), 1.64-1.90 (4H, m), 2.42-2.51 (1H, m), 2.60-2.68 (1H, m), 2.99-3.04 (1H, m), 3.06-3.02 (1H, m), 3.31-3.45 (4H, m), 4.48 (1H, t, J=5.3 Hz), 4.85 (2H, d, J=5.3 Hz), 6.87 (1H, s), 6.99 (1H, dd, J=7.3 Hz, 7.3 Hz), 7.11 (1H, d, J=6.9 Hz), 7.17-7.24 (2H, m), 7.75 (1H, t, J=5.3 Hz), 8.32 (1H, s), 9.11 (1H, t, J=5.6 Hz), 9.32 (1H, s).

### REFERENCE EXAMPLE 66

3-(4-Ethoxycarbonylpiperidino)benzonitrile

By using the same method as described in Reference Example 17, the subject compound is synthesized from 3-fluorobenzonitrile and ethyl isonipecotate.

¹H-NMR (CDCl₃) δ (ppm): 1.28 (3H, t, J=7.3 Hz), 1.77-1.92 (2H, m), 1.95-2.07 (2 h, m), 2.42-2.54 (1H, m), 2.81-2.91 (2H, m), 3.62-3.70 (2H, m), 4.15 (2H, q, J=7.3 Hz), 7.05-7.14 (3H, m), 7.27-7.34 (1H, m).

### REFERENCE EXAMPLE 67

7-Ethylamino-4-[3-(4-hydroxymethylpiperidino)benzylamino]-6-nitroquinazoline

The compound obtained in Reference Example 66 is reduced with aluminum lithium hydride. Thereafter, by using the same method as described in Reference Example 39, the subject compound is obtained from the compound obtained hereinabove through the reduction and 4-chloro-7-ethylamino-6-nitroquinazoline.

¹H-NMR (DMSO-d₆) δ (ppm):1.20-1.50 (5H, m), 1.71-1.77 (2H, m), 2.58-2.66 (2H, m), 3.26-3.43 (5H, m), 3.64-3.69 (2H, m), 4.45 (1H, t, J=5.3 Hz), 4.69 (2H, d, J=5.6 Hz), 6.72-6.86 (3H, m), 6.95 (1H, s), 7.13 (1H, dd, J=7.9 Hz, 7.9 Hz), 7.75 (1H, t, J=5.6 Hz), 8.32 (1H, s), 9.17 (1H, t, J=5.8 Hz), 9.30 (1H, s).

### REFERENCE EXAMPLE 68

2-[4-(2-Hydroxyethyl)piperidino]benzonitrile

By using the same method as described in Reference Example 12, the subject compound is obtained from 2-fluorobenzonitrile and 4-(2-hydroxyethyl)piperidine.

¹H-NMR (CDCl₃) δ (ppm): 1.49-1.62 (6H, m), 1.82-1.87 (2H, m), 2.75-2.83 (2H, m), 3.56-3.61 (2H, m), 3.72-3.77 (2H, m), 6.93-7.01 (2H, m), 7.42-7.56 (2H, m).

### REFERENCE EXAMPLE 69

7-Ethylamino-4-{2-[4-(2-hydroxyethyl)piperidino]benzylamino}-6-nitroquinazoline

The compound obtained in Reference Example 68 is reduced with aluminum lithium hydride. Thereafter, the subject compound is obtained from the compound obtained hereinabove through the reduction and 4-chloro-7-ethylamino-6-nitroquinazoline.

¹H-NMR (DMSO-d₆) δ (ppm): 1.26-1.60 (7H, m), 1.74-1.79 (2H, m), 2.62-2.70 (2H, m), 3.05-3.13 (2H, m), 3.32-3.52 (5H, m), 4.33 (1H, t, J=5.2 Hz), 4.84 (2H, d, J=5.4 Hz), 6.87 (1H, s), 6.93-7.02 (1H, m), 7.12 (1H, d, J=7.4 Hz), 7.19-7.28 (2H, m), 7.74 (1H, t, J=5.2 Hz), 8.32 (1H, s), 9.07 (1H, t, J=5.4 Hz), 9.31 (1H, s).

### REFERENCE EXAMPLE 70

2-(4-Ethoxycarbonyl-1-piperazinyl)benzaldehyde

2-Fluorobenzaldehyde (3.0 g, 24.2 millimole), 1-(ethoxycarbonyl)piperazine (4.2 ml, 28.7 millimole), and calcium carbonate (2.46 g, 24.6 millimole) were reacted in dimethylsulfoxide (12 ml) for 7.5 hours at 120 degrees Celsius. After completion of the reaction, the material which is not dissolved is separated through filtration, water and ethyl acetate were added to the obtained filtrate, and extraction is performed. The organic layer is dried with magnesium sulfate anhydride, concentrated, and oily material is obtained. The obtained oily material is purified using silica gel chromatography (eluted with hexane/ethyl acetate=4/1 to hexane/ethyl acetate=1/1), and the subject compound (4.29 g, 68%) is obtained.

¹H-NMR (CDCl₃) δ (ppm): 1.29 (3H, t, J=7.3 Hz), 3.03-3.07 (4H, m), 3.67-3.71 (4H, m), 4.14 (2H, q, J=7.3 Hz), 7.10-1.19 (2H, m), 7.52 (1H, dd, J=7.6 Hz, 7.9 Hz), 7.83 (1H, d, J=7.6 Hz), 10.35 (1H, s).

### REFERENCE EXAMPLE 71

4-[2-(4-Ethoxycarbonyl-1-piperazinyl)benzylamino]-7-ethylamino-6-nitroquinazoline

The compound obtained in Reference Example 70 (2.5 g, 9.54 millimole) is dissolved with ethanol (25 ml), hydroxylamine hydrochloride (0.862 g, 12.4 millimole) and sodium carbonate (1.32 g, 12.5 millimole) were added, and the solution as stirred for one night at room temperature. After completion of the reaction, the material which is not dissolved is filtered, the filtrate is concentrated, and oily material is obtained. 10%-palladium carbon catalyst (2.30 g) is added to the obtained oily material in methanol (100 ml), and the mixture is stirred under a hydrogen environment at room temperature for one night. After completion of the reaction, the catalyst is separated through filtration using a filter aid. The obtained filtrate is concentrated. The obtained compound (2.1 g), 4-chloro-7-ethylamino-6-nitroquinazoline (2.00 g, 7.92 millimole), and triethylamine (5.50 ml, 40.3 millimole) were stirred for one night in tetrahydrofuran (50 ml). After completion of the reaction, the reaction solution is concentrated, the obtained oily material is purified using silica gel column chromatography (eluted with chloroform/methanol=30/1), and the subject compound (1.53 g, 40%) is obtained.

¹H-NMR (DMSO-d₆) δ (ppm): 1.21(3H, t, J=7.1 Hz), 1.29 (3H, t, J=7.1 Hz), 2.85-2.89 (4H, m), 3.38 (2H, q, J=7.1 Hz), 3.43-3.57 (4H, m), 4.07 (2H, q, J=7.1 Hz), 4.87 (2H, d, J=5.3 Hz), 6.87 (1H, s), 7.05 (1H, dd, J=6.9 Hz, 7.9 Hz), 7.16 (1H, d, J=7.9 Hz), 7.22-7.28 (2H, m), 7.77 (1H, t, J=5.3 Hz), 8.33 (1H, s), 9.14 (1H, t, J=5.3 Hz), 9.32 (1H, s).

### REFERENCE EXAMPLE 72

2-Ethylaminobenzonitrile

By using the same method as described in Reference Example 12, the subject compound is obtained from 2-fluorobenzonitrile and a 70%-ethylamine aqueous solution.

¹H-NMR (CDCl₃) δ (ppm): 1.25 (3H, t, J=7.3 Hz), 3.13-3.21 (2H, m), 4.55 (1H, br), 6.58-6.64 (2H, m), 7.28-7.37 (2H, m).

### REFERENCE EXAMPLE 73

7-Ethylamino-4-(2-ethylaminobenzylamino)-6-nitroquinazoline

The compound obtained in Reference Example 72 is reduced with aluminum lithium hydride. Thereafter, the subject compound is obtained from the compound obtained hereinabove through the reduction and 4-chloro-7-ethylamino-6-nitroquinazoline.

¹H-NMR (DMSO-d₆) δ (ppm) 1.17 (3H, t, J=7.1 Hz), 1.28 (3H, t, J=7.3 Hz), 3.04-3.17 (2H, m), 3.33-3.43 (2H, m), 4.64 (2H, d, J=5.9 Hz), 5.62 (1H, br), 6.52-6.59 (2H, m), 6.87 (1H, s), 7.07-7.15 (2H, m), 7.78 (1H, t, J=5.3 Hz), 8.36 (1H, s), 9.11(1H, t, J=5.9 Hz), 9.25 (1H, s).

### REFERENCE EXAMPLE 74

2-Cyclopentylaminobenzonitrile

By using the same method as described in Reference Example 12, the subject compound is synthesized from 2-fluorobenzonitrile and cyclopentylamine.

¹H-NMR (CDCl₃) δ (ppm): 1.45-1.81 (6H, m), 1.98-2.15 (2H, m), 3.78-3.87 (1H, m), 4.52 (1 h, br), 6.59-6.69 (2H, m), 7.27-7.38 (2H, m).

### REFERENCE EXAMPLE 75

4-(2-Cyclopentylaminobenzylamino)-7-ethylamino-6-nitroquinazoline

The compound obtained in Reference Example 74 is reduced with aluminum lithium hydride. Thereafter, the subject compound is obtained from the compound obtained hereinabove through the reduction and 4-chloro-7-ethylamino-6-nitroquinazoline.

¹H-NMR (DMSO-d₆) δ (ppm): 1.28 (3H, t, J=7.1 Hz), 1.39-1.65 (6H, m), 1.85-1.90 (2H, m), 3.33-3.43 (2H, m), 3.72-3.80 (1H, m), 4.65 (2H, d, J=5.9 Hz), 5.60 (1H, d, J=5.9 Hz), 6.52-6.58 (2H, m), 6.86 (1H, s), 7.07-7.17 (2H, m), 7.76 (1H, t, J=5.3 Hz), 8.35 (1H, s), 9.06 (1H, t, J=5.9 Hz), 9.24 (1H, s).

### REFERENCE EXAMPLE 76

2-Butylaminobenzonitrile

By using the same method as described in Reference Example 12, the subject compound is synthesized from 2-fluorobenzonitrile and butylamine.

¹H-NMR (CDCl₃) δ (ppm): 0.95 (3H, t, J=7.3 Hz), 1.35-1.48 (2H, m), 1.56-1.67 (2H, m), 3.13-3.18 (2H, m), 4.52 (1H, br), 6.59-6.64 (2H, m), 7.31-7.38 (2H, m).

### REFERENCE EXAMPLE 77

4-(2-Butylaminobenzylamino)-7-ethylamino-6-nitroquinazoline

The compound obtained in Reference Example 76 is reduced with aluminum lithium hydride. Thereafter, the subject compound is obtained from the compound obtained hereinabove through the reduction and 4-chloro-7-ethylamino-6-nitroquinazoline.

¹H-NMR (DMSO-d₆) δ (ppm): 0.85 (3H, t, J=7.3 Hz), 1.25-1.37 (5H, m), 1.46-1.57 (2H, m), 2.95-3.05 (2H, m), 3.38 (2H, q, J=6.9 Hz), 4.64 (2H, d, J=5.9 Hz), 5.53 (1H, br), 6.51-6.59 (2H, m), 6.87 (1H, s), 7.06-7.15 (2H, m), 7.77 (1H, t, J=5.3 Hz), 8.35 (1H, s), 9.08 (1H, t, J=5.9 Hz), 9.25 1H, s).

### REFERENCE EXAMPLE 78

2-(1-Pyrrolidinyl)benzonitrile

By using the same method as described in Reference Example 12, the subject compound is synthesized from 2-fluorobenzonitrile and pyrrolidine.

¹H-NMR (CDCl₃) δ (ppm): 1.95-2.02 (4H, m), 3.35-3.61 (4H, m), 6.56-6.66 (2H, m), 7.28-7.34 (1H, m), 7.40-7.44 (1H, m).

### REFERENCE EXAMPLE 79

7-Ethylamino-6-nitro-4-[2-(1-pyrrolidinyl)benzylamino]quinazoline

The compound obtained in Reference Example 78 is reduced with aluminum lithium hydride. Thereafter, the subject compound is obtained from the compound obtained hereinabove through the reduction and 4-chloro-7-ethylamino-6-nitroquinazoline.

¹H-NMR (DMSO-d₆) δ (ppm): 1.29(3H, t, J=7.1 Hz), 1.87-1.92(4H, m), 3.13-3.18(4H, m), 3.34-3.44 (2H, m), 4.76 (2H, d, J=5.3 Hz), 6.82-6.87 (2H, m), 6.98 (1 h, d, J=7.6 Hz), 7.12-7.18 (2H, m), 7.75 (1H, t, J=5.4 Hz), 8.32 (1H, s), 9.11 (1H, t, J=5.3 Hz), 9.33 (1H, s).

### REFERENCE EXAMPLE 80

2-Cyclohexylaminobenzonitrile

By using the same method as described in Reference Example 12, the subject compound is synthesized from 2-fluorobenzonitrile and cyclohexylamine.

¹H-NMR (CDCl₃) δ (ppm): 1.16-1.45 (5H, m), 1.62-1.67 (1H, m), 1.75-1.82 (2H, m), 1.99-2.04 (2H, m), 3.30-3.37 (1H, m), 4.45 (1H, m), 6.57-6.67 (2H, m), 7.30-7.39 (2H, m).

### REFERENCE EXAMPLE 81

4-(2-Cyclohexylaminobenzylamino)-7-ethylamino-6-nitroquinazoline

The compound obtained in Reference Example 80 is reduced with aluminum lithium hydride. Thereafter, the subject compound is obtained from the compound obtained hereinabove through the reduction and 4-chloro-7-ethylamino-6-nitroquinazoline.

¹H-NMR (DMSO-d₆) δ (ppm): 1.05-1.18 (3H, m), 1.20-1.37 (5H, m), 1.55-1.69 (3H, m), 1.85-1.89 (2H, m), 3.15-3.24 (1H, m), 3.33-3.43 (2H, m), 4.64 (2H, d, J=5.6 Hz), 5.50 (1H, d, J=7.3 Hz), 6.50-6.58 (2H, m), 6.87 (1H, s), 7.05-7.18 (2H, m), 7.77 (1H, t, J=5.1 Hz), 8.37 (1H, s), 9.06 (1H, t, J=5.6 Hz), 9.24 (1H, s).

### REFERENCE EXAMPLE 82

2-(4-Ethyl-1-piperazinyl)benzonitrile

By using the same method as described in Reference Example 12, the subject compound is synthesized from 2-fluorobenzonitrile and 4-ethylpiperazine.

¹H-NMR (CDCl₃) δ (ppm): 1.13 (3H, t, J=7.3 Hz), 2.50 (2H, q, J=7.3 Hz), 2.66-2.70 (4H, m), 3.24-3.28 (4H, m), 6.96-7.03 (2H, m), 7.45-7.60 (2H, m).

### REFERENCE EXAMPLE 83

7-Ethylamino-4-[2-(4-ethyl-1-piperazinyl)benzylamino]-6-nitroquinazoline

The compound obtained in Reference Example 82 is reduced with aluminum lithium hydride. Thereafter, the subject compound is obtained from the compound obtained hereinabove through the reduction and 4-chloro-7-ethylamino-6-nitroquinazoline.

¹H-NMR (DMSO-d₆) δ (ppm): 1.03 (3H, t, J=7.3 Hz), 1.28 (3H, t, J=7.1 Hz), 2.39 (2H, q, J=7.3 Hz), 2.50-2.60 (4H, m), 2.85-2.93 (4H, m), 3.36-3.44 (2H, m), 4.84 (2H, d, J=5.3 Hz), 6.87 (1H, s), 7.02 (1H, dd, J=6.6 Hz, 6.9 Hz), 7.15 (1H, d, J=6.9 Hz), 7.16-7.24 (2 h, m), 7.75 (1H, t, J=5.3 Hz), 8.32 (1H, s), 9.08 (1H, br), 9.31 (1H, s).

### REFERENCE EXAMPLE 84

2-(2-Methylpropylamino)benzonitrile

By using the same method as described in Reference Example 12, the subject compound is synthesized from 2-fluorobenzonitrile and isobutylamine.

¹H-NMR (CDCl₃) δ (ppm): 0.99 (6H, d, J=7.6 Hz), 1.80-1.97 (1H, m), 2.97-3.00 (2H, m), 4.61 (1H, br), 6.58-6.64 (2H, m), 7.31-7.37 (2H, m).

### REFERENCE EXAMPLE 85

7-Ethylamino-4-[2-(2-methylpropylamino)benzylamino]-6-nitroquinazoline

The compound obtained in Reference Example 84 is reduced with aluminum lithium hydride. Thereafter, the subject compound is obtained from the compound obtained hereinabove through the reduction and 4-chloro-7-ethylamino-6-nitroquinazoline.

¹H-NMR (DMSO-d₆) δ (ppm): 0.86 (6H, d, J=6.6 Hz), 1.28 (3H, t, J=7.1 Hz), 1.75-1.86 (1h, m), 2.86-2.88 (2H, m), 3.34-3.43 (2H, m), 4.66 (2H, d, J=5.9 Hz), 5.63 (1H, t, J-5.1 Hz), 6.49-6.55 (2H, m), 6.85 (1H, s), 7.05-7.17 (2H, m), 7.76 (1H, t, J=5.3 Hz), 8.35 (1H, s), 9.07 (1H, t, J=5.9 Hz), 9.24 (1H, s).

### REFERENCE EXAMPLE 86

2-(1-Perhydroazepinyl)benzonitrile

By using the same method as described in Reference Example 12, the subject compound is synthesized from 2-fluorobenzonitrile and hexamethyleneimine.

¹H-NMR (CDCl₃) δ (ppm): 1.59-1.64 (4H, m), 1.86-1.87 (4H, m), 3.52, 3.65 (4H, m), 6.61-6.72 (1H, m), 6.84 (1H, d, J=8.6 Hz), 7.29-7.36 (1H, m), 7.45 (1H, d, J=7.9 Hz).

### REFERENCE EXAMPLE 87

4-[2-(1-Perhydroazepinyl)benzylamino]-7-ethylamino-6-nitroquinazoline

The compound obtained in Reference Example 86 is reduced with aluminum lithium hydride. Thereafter, the subject compound is obtained from the compound obtained hereinabove through the reduction and 4-chloro-7-ethylamino-6-nitroquinazoline.

¹H-NMR (DMSO-d₆) δ (ppm): 1.29 (3H, t, J=7.2 Hz), 1.61-1.90 (8H, m), 3.06-3.14 (4H, m), 3.33-3.44 (2H, m), 4.85 (2H, d, J=5.4 Hz), 6.87 (1H, s), 6.93-6.99 (1H, m), 7.09-7.22=(3H, m), 7.74 (1H, t, J=5.4 Hz), 8.32 (1H, s), 9.06 (1H, t, J=5.4 Hz), 9.32 (1H, s).

### REFERENCE EXAMPLE 88

2-(1-Tricyclo[3.3.1.1^{3,7}]decyl)aminobenzonitrile

By using the same method as described in Reference Example 12, the subject compound is synthesized from 2-fluorobenzonitrile and 1-adamantaneamine.

¹H-NMR (CDCl₃) δ (ppm): 1.67-1.76 (7H, m), 1.99-2.01 (6H, m), 2.15-222 (2 h, m), 4.38 (1H, br), 6.64 (1H, dd, J=7.6 Hz), 7.01 (1H, d, J=8.6 Hz), 7.26-7.38 (2H, m).

### REFERENCE EXAMPLE 89

7-Ethylamino-6-nitro-4-[2-(1-tricyclo[3.3.1.1 ³7]decyl)aminobenzylamino]quinazoline

The compound obtained in Reference Example 88 is reduced with aluminum lithium hydride. Thereafter, the subject compound is obtained from the compound obtained hereinabove through the reduction and 4-chloro-7-ethylamino-6-nitroquinazoline.

¹H-NMR (DMSO-d₆) δ (ppm): 1.27 (3H, t, J=7.3 Hz), 1.56-1.70 (6H, m), 1.80-1.90 (6H, m), 2.00-2.09 (3H, m), 3.32-3.43 (2H, m), 4.68 (2H, d, J=5.9 Hz), 4.95 (1H, br), 6.62 (1H, dd, J=7.3 Hz, 7.6 Hz), 6.87 (1H, s), 6.92 (1H, d, J=7.6 Hz), 7.07 (1H, dd, J=7.3 Hz, 7.6 Hz), 7.19 (1H, d, J=7.6 Hz), 7.77 (1H, t, J=5.3 Hz), 8.36 (1H, s), 9.04 (1H, t, J=5.9 Hz), 9.26 (1H, s).

### REFERENCE EXAMPLE 90

2-(4-Hydroxybutylamino)benzonitrile

By using the same method as described in Reference Example 12, the subject compound is synthesized from 2-fluorobenzonitrile and 4-hydroxybutylamine.

¹H-NMR (CDCl₃-CD₃OD)-δ (ppm): 1.62-1.79 (4H, m), 3.20-3.25 (2H, m), 3.67-3.72 (2H, m), 6.61-6.67 (2H, m), 7.33-7.40 (2H, m).

### REFERENCE EXAMPLE 91

7-Ethylamino-4-[2-(4-hydroxybutylamino)benzylamino]-6-nitroquinazoline

The compound obtained in Reference Example 90 is reduced with aluminum lithium hydride. Thereafter, the subject compound is obtained from the compound obtained hereinabove through the reduction and 4-chloro-7-ethylamino-6-nitroquinazoline.

¹H-NMR (DMSO-d₆) δ (ppm): 1.27 (3H, t, J=6.9 Hz), 1.45-1.64 (4H, m), 3.00-3.08 (2H, m), 3.33-3.47 (4H, m), 4.40 (1H, t, J=5.0 Hz), 4.63 (2H, d, J=5.6 Hz), 5.62 (1H, br), 6.52-6.55 (2H, m), 6.85 (1H, s), 7.06-7.05 (2H, m), 7.76 (1H, t, J=5.3 Hz), 8 36 (1H, s), 9.08 (1H, t, J=5.6 Hz), 9.24 (1H, s).

### REFERENCE EXAMPLE 92

6-Nitro-7-propylamino-4(3H)-quinazoline

7-Chloro-6-nitro-4(3H)-quinazoline (5 g, 22.2 millimole) is heated in dimethylsulfoxide (15 ml) at 140 degrees Celsius. At the same temperature, n-propylamine (5 ml, 60.8 millimole) is added, and the solution is stirred for 30 minutes. After completion of the reaction, the solution is cooled. Precipitated crystal is obtained through filtration, washed with methanol, dried, and the subject compound is obtained.

¹H-NMR (DMSO-d₆) δ (ppm): 0.98 (3H, t, J=7.4 Hz), 1.63-1.74 (2H, m), 3.31-3.39 (2H, m), 6.91 (1H, s), 8.08 (1H, s), 8.15 (1H, t, J=5.3 Hz), 8.73 (1H, s), 12.00 (1H, br).

### REFERENCE EXAMPLE 93

4-[2-(4-Hydroxymethylpiperidino)benzylamino]-6-nitro-7-propylaminoquinazoline

The compound obtained in Reference Example 92 (1.2 g, 4.84 millimole) is suspended with phosphorus oxychloride (11 ml, 118 millimole), and the solution is heated under an argon gas environment for 2 hours at 110 degrees Celsius to make a uniform solution. After dissipation of the source materials for the reaction is confirmed, the phosphorus oxychloride which did not react is removed under reduced pressure. After the solution is azeotropically boiled with toluene, the obtained oily material is dissolved with the minimum necessary amount of tetrahydrofuran. The obtained tetrahydrofuran solution is poured over ice water in which a sufficient amount of sodium hydrogencarbonate is added, and extraction is performed with ethyl acetate. The organic layer is dried with the desiccant (magnesium sulfate anhydride), and then the desiccant is separated through filtration. The filtrate is concentrated under reduced pressure, and an orange color solid (1.39 g) is obtained.

The compound obtained in Reference Example 13 is reduced with aluminum lithium hydride. Thereafter, the subject compound is obtained from the compound obtained hereinabove through the reduction and the above orange color solid (1.39 g).

¹H-NMR (DMSO-d₆) δ (ppm): 1.00 (3H, t, J=7.3 Hz), 1.26-1.63 (3H, m), 1.66-1.79 (4H, m), 2.62-2.70 (2H, m), 3.06-3.15 (2H, m), 3.29-3.36 (4H, m), 4.47 (1H, t, J=5.1 Hz), 4.84 (2H, d, J=5.3 Hz), 6.87 (1H, s), 6.96-7.09 (1H, m), 7.11-7.25 (3H, m), 7.83 (1H, t, J=5.4 Hz), 8.33 (1H, s), 9.11 (1H, t, J=5.3 Hz), 9.32 (1H, s).

### REFERENCE EXAMPLE 94

2-(4-Methyl-1-homopiperazinyl)benzonitrile

By using the same method as described in Reference Example 12, the subject compound is synthesized from 2-fluorobenzonitrile and 4-methylhomopiperazine.

¹H-NMR (CDCl₃) δ (ppm): 2.01-2.10 (2H, m), 2.41 (3H, s), 2.63-2.67 (2H, m), 2.80-2.84 (2H, m), 3.59-3.68 (2H, m), 3.69-3.71 (2H, m), 6.71-6.78 (1H, m), 6.85 (1H, d, J=8.6 Hz), 7.33-7.45 (1H, m), 7.47 (1H, d, J=7.9 Hz).

### REFERENCE EXAMPLE 95

7-Ethylamino-4-[2-(4-methyl-1-homopiperazinyl)benzylamino]-6-nitroquinazoline

The compound obtained in Reference Example 94 is reduced with aluminum lithium hydride. Thereafter, the subject compound is obtained from the compound obtained hereinabove through the reduction and 4-chloro-7-ethylamino-6-nitroquinazoline.

¹H-NMR (DMSO-d₆) δ (ppm): 1.31 (3H, t, J=7.1 Hz), 2.10-2.27 (2H, m), 2.81 (3H, s), 3.09-3.18 (2H, m), 3.20-3.42 (8H, m), 4.87 (2H, d, J=4.3 Hz), 6.87 (1H, s), 7.04-7.10 (1H, m), 7.21-7.30 (3H, m), 7.76 (1H, t, J=5.1 Hz), 8.33 (1H, s), 9.17 (1H, t, J=4.3 Hz), 9.36 (1H, s).

### REFERENCE EXAMPLE 96

2-[2-(2-Hydroxyethoxy)ethylamino]benzonitrile

By using the same method as described in Reference Example 12, the subject compound is synthesized from 2-fluorobenzonitrile and 2-(2-aminoethoxy)ethanol.

¹H-NMR (CDCl₃) δ (ppm): 2.50 (1H, br), 3.34-3.47 (3H, m), 3.60-3.69 (2H, m), 3.70-3.80 (4H, m), 6.67-6.70 (2H, m), 7.35-7.42 (2H, m).

### REFERENCE EXAMPLE 97

7-Ethylamino-4-{2-[2-(2-hydroxyethoxy)ethylamino]benzylamino}-6-nitroquinazoline

The compound obtained in Reference Example 96 is reduced with aluminum lithium hydride. Thereafter, the subject compound is obtained from the compound obtained hereinabove through the reduction and 4-chloro-7-ethylamino-6-nitroquinazoline.

¹H-NMR (DMSO-d₆) δ (ppm): 1.27 (3H, t, J=7.1 Hz), 3.21-3.30 (2H, m), 3.36-3.45 (6H, m), 3.58 (2 h, t, J=5.8 Hz), 4.56 (1H, br), 4.63 (2H, d, J=5.8 Hz), 5.67 (1H, br), 6.55-6.61 (2H, m), 6.87 (1H, s), 7.08-7.16 (2H, m), 7.77 (1H, t, J=5.3 Hz), 8.38 (1H, s), 9.10 (1H, t, J=5.9 Hz), 9.26 (1H, s).

### REFERENCE EXAMPLE 98

7-Methylamino-6-nitro-4(3H)-quinazoline

7-Chloro-6-nitro-4(3H)-quinazoline (6.06 g, 26.9 millimole) is heated in dimethylsulfoxide (20 ml) at 110 degrees Celsius. At the same temperature, a 40%-methylamineaqueous solution (80 ml) is added, and the solution is stirred for 30 minutes. After completion of the reaction, the solution is cooled. Precipitated crystal is obtained through filtration, washed with methanol, and then dried (5.38 g).

¹H-NMR (DMSO-d₆) δ (ppm): 3.04 (3H, d, J=4.0 Hz), 6.90 (1H, s), 8.24 (1H, s), 8.28 (1H, br), 8.81 (1H, s), 12.00 (1H, br).

### REFERENCE EXAMPLE 99

4-[2-(4-Hydroxymethylpiperidino)benzylamino]-7-methylamino-6-nitroquinazoline

The compound obtained in Reference Example 98 (7.0 g, 31.8 millimole) and N,N-diisopropylethylamine (10 ml, 57.4 millimole) were suspended with phosphorus oxychloride (80 ml, 858 millimole), and the suspension is heated for 2 hours under an argon gas environment at 110 degrees Celsius to make a uniform solution. After dissipation of the source materials for the reaction is confirmed, the phosphorus oxychloride which does not react is removed under reduced pressure. After the solution is azeotropically boiled with toluene, the obtained oily material is dissolved with the minimum necessary amount of tetrahydrofuran. The obtained tetrahydrofuran solution is poured into ice water in which a sufficient amount of sodium hydrogencarbonate is added, and extraction is performed with ethyl acetate. After the organic layer is dried with the desiccant (magnesium sulfate anhydride), the desiccant is separated through filtration. The filtrate is concentrated under reduced pressure, and an orange colored solid is obtained.

The compound which is obtained in Reference Example 13 is reduced with aluminum lithium hydride, then the subject compound is obtained from the compound obtained hereinabove through the reduction and the above-obtained orange colored solid.

¹H-NMR (DMSO-d₆) δ (ppm): 1.28-1.56 (3H, m), 1.71-1.80 (2H, m), 2.52-2.73 (2H, m), 2.99 (3H, d, J=4.6 Hz), 3.01-3.12 (2H, m), 3.23-3.42 (2H, m), 4.47 (1H, br), 4.84 (2H, d, J=5.3 Hz), 6.82 (1H, s), 6.96-7.02 (1H, m), 7.11-7.25 (3H, m), 7.93 (1H, br), 8.28 (1H, s), 9.08 (1H, t, J=5.3 Hz), 9.31 (1H, s).

### REFERENCE EXAMPLE 100

2-(2-Furilmethylamino)benzonitrile

By using the same method as described in Reference Example 12, the subject compound is synthesized from 2-fluorobenzonitrile and 2-(aminomethyl)furan.

¹H-NMR (CDCl₃) δ (ppm): 4.41 (2H, s), 4.60 (1H, br), 6.25-6.27 (1H, m), 6.32-6.34 (1H, m), 6.68-6.77 (2H, m), 7.35-7.41 (3H, m).

### REFERENCE EXAMPLE 101

7-Ethylamino-4-[2-(2-furilmethylamino)benzylamino]-6-nitroquinazoline

The compound obtained in Reference Example 100 is reduced with aluminum lithium hydride. Thereafter, the subject compound is obtained from the compound obtained hereinabove through the reduction and 4-chloro-7-ethylamino-6-nitroquinazoline.

¹H-NMR (CDCl₃) δ (ppm): 1.39 (3H, t, J=7.1 Hz), 3.31-3.41(2H, m), 4.28 (2H, s), 4.84 (2H, s), 6.18-6.20 (1H, m), 6.9-6.32 (1H, m), 6.68-6.74 (2H, m), 7.03 (1H, s), 7.19-7.31 (4H, m), 7.73 (1H, br), 8.24 (1H, s), 8.78 (1H, s), 9.26 (1H, s).

### REFERENCE EXAMPLE 102

2-(1,2,3,4-Tetrahydroisoquinoline-2-yl)benzonitrile

By using the same method as described in Reference Example 12, the subject compound is synthesized from 2-fluorobenzonitrile and 1,2,3,4-tetrahydroisoquinoline.

¹H-NMR (CDCl₃) δ (ppm): 3.07-3.11 (2H, m), 3.65-3.69 (2H, m), 4.41 (2H, s), 6.93-6.99 (1H, m), 7.05-7.21 (5H, m), 7.43-7.50 (1H, m), 7.58 (1H, dd, J=1.7 Hz, 7.6 Hz).

### REFERENCE EXAMPLE 103

7-Ethylamino-6-nitro-4-[2-(1,2,3,4-tetrahydroisoquinoline-2-yl]benzylamino)quinazoline

The compound obtained in Reference Example 102 is reduced with aluminum lithium hydride. Thereafter, the subject compound is obtained from the compound obtained hereinabove through the reduction and 4-chloro-7-ethylamino-6-nitroquinazoline.

¹H-NMR (CDCl₃) δ (ppm): 1.38 (3H, t, J=7.3 Hz), 3.10-3.15 (2H, m), 3.31-3.42 (4H, m), 4.21 (2H, s), 5.01 (2H, d, J=4.0 Hz), 6.98 (1H, s), 7.00-7.20 (5H, m), 7.25-7.39 (3H, m), 7.65 (1H, br), 8.11 (1H, t, J=4.0 Hz), 8.41 (1H, s), 8.47 (1H, s).

### REFERENCE EXAMPLE 104

2-(2-Tetrahydrofurilmethylamino)benzonitrile

By using the same method as described in Reference Example 12, the subject compound is synthesized from 2-fluorobenzonitrile and 2-(tetrahydrofuril)methylamine.

¹H-NMR (CDCl₃) δ (ppm): 1.60-1.73 (1H, m), 1.86-2.11 (3 h, m), 3.17-3.24 (1H, m), 3.31-3.47 (1H, m), 3.75-3.81 (1H, m), 3.83-3.96 (1H, m), 4.09-4.19 (1H, m), 4.39 (1H, br), 6.63-6.72 (2H, m), 7.27-7.40 (2H, m).

### REFERENCE EXAMPLE 105

7-Ethylamino-6-nitro-4-[2-(2-tetrahydrofurilmethylamino)benzylamino]quinazoline

The compound obtained in Reference Example 104 is reduced with aluminum lithium hydride. Thereafter, the subject compound is obtained from the compound obtained hereinabove through the reduction and 4-chloro-7-ethylamino-6-nitroquinazoline.

¹H-NMR (CDCl₃) δ (ppm): 1.40 (3H, t, J=7.3Hz), 1.53-1.63 (1H, m), 1.80-2.01 (3H, m), 3.03-3.11 (1H, m), 3.20-3.26 (1H, m), 3.32-3.43 (2H, m), 3.64-3.78 (2H, m), 4.02-4.09 (1H, m), 4.83 (2H, br), 5.20 (1H, s), 6.66-6.73 (2H, m), 6.80 (1H, s), 7.02 (1H, s), 7.19-7.26 (2H, m), 7.72 (1H, br), 8.53 (1H, s), 8.79 (1H, s).

### REFERENCE EXAMPLE 106

2-(2-Thienylmethylamino)benzonitrile

By using the same method as described in Reference Example 12, the subject compound is synthesized from 2-fluorobenztolyl and 2-aminomethylthiophene.

¹H-NMR (CDCl₃) δ (ppm): 4.61 (2H, s), 5.60 (1H, br), 6.69-6.75 (2H, m), 6.95-7.03 (2H, m), 7.24 (1H, d, J=7.6 Hz), 7.34-7.43 (2H, m).

### REFERENCE EXAMPLE 107

7-Ethylamino-6-nitro-4-[2-(2-thienylmethylamino)benzylamino]quinazoline

The compound obtained in Reference Example 106 is reduced with aluminum lithium hydride. Thereafter, the subject compound is obtained from the compound obtained hereinabove through the reduction and 4-chloro-7-ethylamino-6-nitroquinazoline.

¹H-NMR (CDCl₃) δ (ppm): 1.39 (3H, t, J=7.3 Hz), 3.29-3.39 (2H, m), 4.47 (2H, d, J=4.3 Hz), 4.83 (2H, d. J=5.9 Hz), 6.08 (1H, br), 6.40 (1H, t, J=4.3 Hz), 6.69-6.75 (2H, m), 6.91-6.95 (3H.m), 7.17-7.26 (3H.m), 7.68 (1H, t, J=5.9 Hz), 8.11 (1H, s), 8.79 (1H, s).

### REFERENCE EXAMPLE 108

2-(2-Phenylaminoethylamino)benzonitrile

By using the same method as described in Reference Example 12, the subject compound is synthesized from 2-fluorobenzonitrile and N-phenylethylenediamine.

¹H-NMR (CDCl₃, ) δ (ppm): 3.41-3.65 (4H, m), 3.80 (1H, br), 4.79 (1H, br), 6.67-6.80 (5H, m), 7.16-7.24 (2H, m), 7.34-7.41 (2H, m).

### REFERENCE EXAMPLE 109

7-Ethylamino-6-nitro-4-[2-(2-phenylaminoethylamino)benzylamino]quinazoline

The compound obtained in Reference Example 108 is reduced with aluminum lithium hydride. Thereafter, the subject compound is obtained from the compound obtained hereinabove through the reduction and 4-chloro-7-ethylamino-6-nitroquinazoline.

¹H-NMR (CDCl₃,) δ (ppm): 1.36 (3H, t, J=7.3HZ), 3.23-3.43 (7H. M), 4.80 (2H, d, J=4.6 Hz), 5.60 (1H, br), 6.45-6.49 (2H, M), 6.59-6.69 (3H, m), 6.80-6.92 (2H, m).7.02-7.09 (2H, m), 7.17-7.25 (2H, m), 7.65 (1H, t, J=4.6HZ), 8.35 (1H, s), 8.70(1H, s).

### REFERENCE EXAMPLE 110

2-(4-Hydroxymethylpiperidino)benzonitrile

A solution is prepared in which aluminum lithium hydride (0.90 g, 23.7 millimole) is suspended with tetrahydrofuran (20 ml). The solution is stirred under ice-chilled conditions under an argon gas environment. A solution, in which 2-(4-ethoxycarbonylpiperidino)benzonitrile (6.00 g, 23.3 millimole) obtained in Reference Example 13 is dissolved with tetrahydrofuran (50 ml) solution, is dripped little by little to this solution. After completion of dripping, the solution is stirred for 1 hour at the same temperature. After completion of the reaction, the reaction solution is cooled, sodium sulfate decahydrate is added little by little until foaming stopped. Thereafter, the material which is not dissolved is separated through filtration, the filtrate is concentrated under reduced pressure, and the subject compound (5.0 g) is obtained.

¹H-NMR (CDCl₃) δ (ppm): 1.43-1.74 (4H, m), 1.83-1.92 (2H, m), 2.77-2.93 (2H, m), 3.53-3.69 (4H, m), 6.94-7.04 (2H, m), 7.43-7.53 (1H, m), 7.55 (1H, d, J=7.9 Hz).

### REFERENCE EXAMPLE 111

2-(4-Methoxymethylpiperidino)benzonitrile

The compound (2.94 g, 13.6 millimole) obtained in Reference Example 110 is dissolved with N,N-dimethylformamide (14 ml) under ice-chilled conditions, sodium hydride (40%-in oil, 0.83 g, 21 millimole) is added, and the solution is stirred for 2 hours at the same temperature. Thereafter, methyl iodide (1.3 ml, 20.9 millimole) is added. It is then stirred for 20 minutes at 60-degrees Celsius. After completion of the reaction, the reaction solution is poured into ice water, and extraction is performed with ether. The organic layer is dried (magnesium sulfate anhydride). Thereafter, the organic layer is concentrated, purified using column chromatography (chloroform/ethyl acetate=10/1), and the subject compound (3.01 g, 96%) is obtained.

¹H-NMR (CDCl₃) δ (ppm): 1.43-1.58 (2H, m), 1.68-1.79(1H, m), 1.80-1.90(2H, m), 2.75-2.84(2H, m), 3.29 (2H, d, J=6.3 Hz), 3.36 (3H, s), 3.53-3.62(2H, m), 6.93-7.12 (2H, m), 7.41-7.50 (1H, m), 7.5 3 (1H, d, J=7.6 Hz).

### REFERENCE EXAMPLE 112

7-Ethylamino-4-[2-(4-methoxymethylpiperidino)benzylamino]-6-nitroquinazoline

The compound obtained in Reference Example 111 is reduced with aluminum lithium hydride. Thereafter, the subject compound is obtained from the compound obtained hereinabove through the reduction and 4-chloro-7-ethylamino-6-nitroquinazoline.

¹H-NMR (DMSO-d₆) δ (ppm) 1.29 (3H, t, J=7.1 Hz), 1.31-1.42 (2H, m), 1.67-1.77 (3H, m), 2.62-2.72(2H, m), 3.05-3.10(2H, m), 3.18(2H, d, J=4.3 Hz), 3.25(3H, s), 3.34-3.44 (2H, m), 4.83 (2H, d, J=5.3 Hz), 6.87(1H, s), 7.00(1H, dd, J=7.3 Hz, 7.3 Hz), 7.13(1H, d, J=7.9 Hz), 7.19-7.25(2H.m), 7.76(1H, J=5.4 Hz), 8.33(1H, s), 9.10(1H, t, J=5.3 Hz), 9.31(1H, s).

### REFERENCE EXAMPLE 113

2-(4-Ethoxymethylpiperidino)benzonitrile

By using the same method as described in Reference Example 111, the subject compound is obtained from the compound obtained in Reference Example 110 and ethyl iodide.

¹H-NMR (CDCl₃) δ (ppm): 1.18 (3H, t, J=7.1 Hz), 1.43-1.57 (2H, m), 1.70-1.81 (1H, m), 1.86-1.92 (2H, m), 2.74-2.84 (2H, m), 3.33 (2H, d, J=6.6 Hz), 3.49 (2H, q, J=7.1 Hz), 3.57-3.76 (2H, m), 6.92-7.02 (2H, m), 7.44(1H, dd, J=7.3 Hz, 8.6 Hz), 7.53 (1H, d, J=7.6 Hz).

### REFERENCE EXAMPLE 114

4-[2-(4-Ethoxymethylpiperidino)benzylamino]-7-ethylamino-6-nitroquinazoline

The compound obtained in Reference Example 113 is reduced with aluminum lithium hydride. Thereafter, the subject compound is obtained from the compound obtained hereinabove through the reduction and 4-chloro-7-ethylamino-6-nitroquinazoline.

¹H-NMR (DMSO-d₆) δ (ppm): 1.1 2 (3H, t, J=6.9 Hz), 1.26-1.42 (5H, m), 1.60-1.79 (3H, m), 2.62-2.72 (2H, m), 3.06-3.15 (2H, m), 3.27 (2H, d, J=5.9 Hz), 3.34-3.46 (4H, m), 4.84 (2H, d, J=5.0 Hz), 6.87 (1H, s), 7.00 (1H, dd, J=7.3 Hz, 7.6 Hz), 7.13 (1H, d, J=7.9 Hz), 7.1 9-7.25 (2H, m), 7.75 (1H, t, J=5.3 Hz), 8.33 (1H, s), 9.09 (1H, t, J=5.0 Hz), 9.31 (1H, s).

### REFERENCE EXAMPLE 115

4-(2-Aminobenzylamino)-7-ethylamino-6-nitroquinazoline

4-Chloro-7-ethylamino-6-nitroquinazoline (5.00 g, 19.8 millimole), triethylamine (8.3 ml, 60.9 millimole), and 2-aminobenzylamine (2.9 g, 23.8 millimole) were stirred in tetrahydrofuran for one night at room temperature. After completion of the reaction, the solution is concentrated under reduced pressure, the obtained solid is washed with methanol, and the subject compound (5.23 g, 78%) is obtained.

¹H-NMR (DMSO-d₆) δ (PPM): 1.28 (3H, t, J=7.1 Hz), 3.33-3.45 (4H, m), 4.59 (2H, br), 5.27(1H, br), 6.52(1H, dd, J=7.3 Hz, 7.6 Hz), 6.64(1H, d, J=7.9 Hz), 6.85(1H, s), 6.96(1H, dd, J=7.3 Hz, 7.9 Hz), 7.08(1H, d.J=7.6 Hz), 7.77(1H, t, J=5.3 Hz), 8.34 (1H, s), 9.27 (1H, s).

### REFERENCE EXAMPLE 116

4-[2-(tert-Butoxycarbonylamino)benzylamino]-7-ethylamino-6-nitroquinazoline

The compound (2.50 g, 7.40 millimole) obtained in Reference Example 115 is suspended with tetrahydrofuran (150 ml). Then, di-tert-butyldicharbonate (7.2 ml, 31.3 millimole) and triethylamine (3.80 ml, 27.3 millimole) were added, and the solution is stirred for one night at room temperature. After completion of the reaction, the reaction solution is concentrated under reduced pressure, the obtained solid is purified with column chromatography (chloroform/ethyl acetate 5/1), and the subject compound (2.80 g, 87%) is obtained.

¹H-NMR (CDCl₃) δ (ppm): 1.37 (3H, 1, J=7.3 Hz), 1.56 (9H, s), 3.31-3.39(2H, m), 4.81 (2H, d, J=5.6 Hz), 5.62 (1H, br), 6.95-7.10 (2H, m), 7.15-7.30 (2H, m), 7.38 (1H, d, J=7.3 Hz), 7.73-7.88 (2H, m), 8.57 (1H, s), 8.93 (1H, s).

### REFERENCE EXAMPLE 117

8-[2-(tert-Butoxycarbonylamino)benzylamino]-3-ethyl-2,3-dihydro-1H-imidazo[4.5-g]quinazoline-2-thione

The compound (1.50 g, 3.42 millimole) obtained in Reference Example 116 is suspended with the mixture solvent of methanol (50 ml) and tetrahydrofuran (75 ml). Then, 10%-palladium-carbon catalyst (0.2 g) is added, and the solution is stirred for one night under a hydrogen gas environment. After completion of the reaction, the catalyst is separated through filtration by using filter aid. Carbon disulfide (10 ml, 166 millimole) and triethylamine (10.0 ml, 71.8 millimole) were added to the filtrate, and the solution is stirred for one night at room temperature. After completion of the reaction, the reaction solution is concentrated under reduced pressure. The obtained solid is separated through filtration, washed with methanol, then purified through re-crystallization (N,N-dimethylformamide-water), and the subject compound (1.04 g, 68%) is obtained.

¹H-NMR (DMSO-d₆) δ (ppm): 1.30 (3H, t, J=6.9 Hz), 1.50 (9H, s), 4.35 (2H, d, J=6.9 Hz), 4.69 (2H.d, J=5.9 Hz), 7.03 (1H, dd, J=7.3 Hz, 7.6 Hz), 7.23 (1H, dd, J=7.3 Hz, 8.2 Hz), 7.37 (1H, d, J=7.6 Hz), 7.62 (1H, s), 7.73 (1H, d, J=8.2 Hz), 8.01 (1H, s), 8.44 (1H, s), 8.98 (1H, t, J=5.9 Hz), 10.28 (1H, br), 13.30 (1H, s).

### REFERENCE EXAMPLE 118

2-Cyclobutylaminobenzonitrile

By using the same method as described in Reference Example 12, the subject compound is synthesized from 2-fluorobenzonitrile and cyclobutylamine.

¹H-NMR (CDCl₃) δ (ppm): 1.75-1.99 (4H, M), 2.40-2.50(2H, m), 3.91-4.01 (1H, m), 4.50 (1H, br), 6.57(1H, d, J=8.6 Hz), 6.65(1H, dd, J=7.3 Hz, 7.9 Hz), 7.32-7.40 (2H, m).

### REFERENCE EXAMPLE 119

4-(2-Cyclobutylaminobenzylamino)-7-ethylamino-6-nitroquinazoline

The compound obtained in Reference Example 118 is reduced with aluminum lithium hydride. Thereafter, the subject compound is obtained from the compound obtained hereinabove through the reduction and 4-chloro-7-ethylamino-6-nitroquinazoline.

¹H-NMR (CDCl₃) δ (ppm): 1.38 (3H, t, J=7.3 Hz), 1.71-1.89 (4H, m), 2.30-2.41 (2H, m), 3.33-3.38 (2H, m), 3.81-3.89 (1H, m), 4.84 (2H, br), 5.65 (1H, br), 6.51 (1H, d, J=7.9 Hz), 6.65 (1H, dd, J=7.3 Hz, 7.6 Hz), 6.80 (1H, br), 7.01 (1H, s), 7.15-7.24 (2H, m), 7.73 (1H, 1, J=4.8 Hz), 8.53 (1H, s), 8.80 (1H, s).

### REFERENCE EXAMPLE 120

2-(exo-2-Bicyclo[2.2.1]heptylamino)benzonitrile

By using the same method as described in Reference Example 12, the subject compound is synthesized from 2-fluorobenzonitrile and exo-2-aminonorbornane.

¹H-NMR (CDCl₃) 6 (PPM): 1.13-1.32 (4H, m), 1.46-1.61 (3H, m), 1.81-1.90 (1H, m), 2.26-2.38 (2H, m), 3.26-3.30 (1H, m), 4.39 (1H, br), 6.60-6.67 (2H, m), 7.2 8-7.39 (2H, m).

### REFERENCE EXAMPLE 121

4-[2-(Exo-2-bicyclo[2.2.1 ]heptylamino)benzylamino]-7-ethylamino-6-nitroquinazoline

The compound obtained in Reference Example 120 is reduced with aluminum lithium hydride. Thereafter, the subject compound is obtained from the compound obtained hereinabove through the reduction and 4-chloro-7-ethylamino-6-nitroquinazoline.

¹H-NMR (CDCl₃) δ (ppm): 1.04-1.27 (5H, m), 1.34-1.55 (5H, m), 1.71-1.78 (1H, m), 2.17-2.23 (2H, m), 2.40 (1H, br), 3.19-3.21 (1H, m), 3.28-3.35 (2H, m), 4.73-4.91 (2H, m), 6.50-6.64 (2H, m), 6.85 (1H, br), 6.96 (1H, s), 7.16-7.21 (2H, m), 7.70 (1H, t, J=4.6 Hz), 8.49(1H, s), 8.78(1H, s).

### EXAMPLE 1

3-Ethyl-8-(2-methylaminobenzylamino)-2,3-dihydro-1H-imidazo[4,5-g]quinazoline-2-one 2 Hydrochloride (Compound 1)

7-Ethylamino-4-(2-methylaminobenzylamino)-6-nitroquinazoline (4.00 g, 11.4 millimole) obtained in Reference Example 28 is suspended with the mixture solvent of methanol (100 ml) and tetrahydrofuran (150 ml). 10%-Palladium carbon catalyst (0.40 g) is added to this suspension, and the solution is stirred for one night at room temperature under a hydrogen gas environment. After the reaction is completed, the catalyst is separated through filtration by using filter aid, and the obtained filtrate is divided into halves. One of the divided halves of the filtrate is concentrated under reduced pressure. The obtained concentrated residuum is dissolved with acetonitrile (100 ml), and N,N′-carbonyldiimidazole (2.80 g, 17.3 millimole) is added. Then, the solution is stirred for 5 hours while the solvent is refluxed. After the reaction is completed, the solvent is removed under reduced pressure, and the obtained residuum is purified with silica gel column chromatography (eluted with chloroform/methanol=100). Thus, the free base of the subject compound (1.31 g) is obtained. The obtained free base is suspended with methanol (50 ml), and a sufficient amount of 4N hydrochloric acid-ethyl acetate is added under ice-chilled conditions. The solution is concentrated to half under reduced pressure, the precipitated crystal is washed with ether-ethanol, and the subject compound (1.48 g, 62%) is obtained.

¹H-NMR (DMSO-d₆) δ (ppm): 1.28 (3H, t, J=7.2 Hz), 2.92 (3H, s), 3.95(2H, J=7.2 Hz), 4.60 (1H, br), 4.90 (2H, d, J=4.0 Hz), 6.94-7.05 (2H, m), 7.26-7.35 (2H, m), 7.49 (1H, s), 8.22 (1H, s), 8.84 (1H, s), 10.61(1H, 1, J=4.0 Hz), 11.98 (1H, s).

### EXAMPLE 2

3-Ethyl-8-(4-methylaminobenzylamino)-2,3-dihydro-1H-imidazo[4,5-g]quinazoline-2-one 2 hydrochloride (Compound 2)

Using the compound obtained in Reference Example 29 as source material, the subject compound is obtained by using the same method as described in Example 1.

¹H-NMR (DMSO-d₆) δ (ppm): 1.24 (3H, t, J=7.3 Hz), 2.80 (3H, s), 3.60 (1H, br), 3.91 (2H, q, J=7.31 Hz), 4.98 (2H, d, J=5.6 Hz), 7.29 (2H, d, J=8.3 Hz), 7.45 (2H, d, J=8.3 Hz), 7.50 (1H, s), 8.19 (1H, s), 8.78 (1H, s), 10.62 (1H, t, J=5.6 Hz), 12.02 (1H, s).

### EXAMPLE 3

8-(4-Benzylaminobenzylamino)-3-ethyl-2,3-dihydro-1H-imidazo[4,5-g]quinazoline-2-one 2 Hydrochloride (Compound 3)

Using the compound obtained in Reference Example 30 as source material, the subject compound is obtained by using the same method as described in Example 1.

¹H-NMR (DMSO-d₆) δ (ppm): 1.27 (3H, t, J=7.2 Hz), 3.94 (2H, q, J=7.2 Hz), 4.37(2H, s), 4.83 (2H, d, J=5.4 Hz), 6.90-7.00 (2H, m), 7.20-7.45 (8H, m), 7.48 (1H, s), 8.15 (1H, s), 8.78 (1H, S), 10.46 (1H, 1, J=5.4 Hz), 11.98 (1H, s).

### EXAMPLE 4

3-Ethyl-8-(4-isopropylaminobenzylamino)-2,3-dihydro-1H-imidazo[4,5-g]quinazoline-2-one 2 Hydrochloride (Compound 4)

Using the compound obtained in Reference Example 31 as source material, the subject compound is obtained by using the same method as described in Example 1.

¹H-NMR (DMSO-d₆) δ (ppm): 1.23-1.31 (9H, m), 3.61-3.66 (2H, m), 3.95 (2H, q, J=7.3 Hz), 4.94 (2H, d, J=5.6 Hz), 7.41-7.53 (5H, m), 8.17 (1H, s), 8.81 (1H.s), 10.53 (1H, t, J=5.6 Hz), 11.99 (1H, s).

### EXAMPLE 5

3-Ethyl-8-(4-propylaminobenzylamino)-2,3-dihydro-1H-imidazo[4,5-g]quinazoline-2-one 2 Hydrochloride (Compound 5)

Using the compound obtained in Reference Example 32 as source material, the subject compound is obtained by using the same method as described in Example 1.

¹H-NMR (DMSO-d₆) δ (ppm): 0.93 (3H, t, J=7.4 Hz), 1.27 (3H, t, J=7.1 Hz), 1.58-1.71 (2H, m), 3.10-3.16 (2H, m), 3.60 (1H, br), 3.94 (2H, q, J=7.1 Hz), 4.90 (2H, d, J=5.6 Hz), 7.29-7.35 (2H, m), 7.43-7.48 (3H, m), 8.17 (1H, s), 8.81 (1H.s), 10.54(1H, t, J=5.6 Hz), 12.01 (1H, s).

### EXAMPLE 6

3-Ethyl-8-(4-ethylaminobenzylamino)-2,3-dihydro-1H-imidazo[4,5-g]quinazoline-2-one 2 Hydrochloride (Compound 6)

Using the compound obtained in Reference Example 33 as source material, the subject compound is obtained by using the same method as described in Example 1.

¹H-NMR (DMSO-d₆) δ (ppm): 1.20-1.30 (6H, m), 3.23 (2H, q, J=6.9 Hz), 3.61 (1H, br), 3.95 (2H, q, J=7.3 Hz), 4.91 (2H, d, J=5.6 Hz), 7.29-7.40 (2H, m), 7.44-7.50 (3H, m), 8.15 (1H, s), 8.81 (1H, s), 10.49 (1H, 1, J=5.6 Hz), 11.99 (1H, s).

### EXAMPLE 7

3-Ethyl-8-[2-(2-morpholinoethylamino)benzylamino]-2,3-dihydro-1H-imidazo[4,5-g]quinazoline-2-one 3 Hydrochloride (Compound 7)

Using the compound obtained in Reference Example 34 as source material, the subject compound is obtained by using the same method as described in Example 1.

¹H-NMR (DMSO-d₆) δ (ppm): 1.27 (3H, t, J=6.9 Hz), 3.10-3.30 (2H, m), 3.35-3.60 (6H, m), 3.80-4.00 (6H, m), 4.81 (2H, d, J=5.4 Hz), 5.70 (1H, br), 6.63 (1H, dd, J=7.4 Hz, 7.4 Hz), 6.74 (1H, d, J=7.9 Hz), 7.14 (1H, dd, J=7.4 Hz, 7.9 Hz), 7.22 (1H, d, J=7.4 Hz), 7.51 (1H, s), 8.31(1H, s), 8.83 (1H, s), 10.65 (1H, t, J=5.4 Hz), 12.00 (1H, s).

### EXAMPLE 8

3-Ethyl-8-[2-(3-morpholinopropylamino)benzylamino]-2,3-dihydro-1H-imidazo[4,5-g]quinazoline-2-one 3 Hydrochloride (Compound 8)

Using the compound obtained in Reference Example 35 as source material, the subject compound is obtained by using the same method as described in Example 1.

¹H-NMR (DMSO-d₆) δ (ppm): 1.29 (3H, t, J=7.2 Hz), 2.20-2.28 (2H, m) 3.05-3.20 (2H, m), 3.27-3.51 (6H, m), 3.85-4.00 (6H, m), 4.89 (2H, d, J=5.4 Hz), 5.70 (1H, br), 6.82 (1%, dd, J=6.9 Hz, 6.9 Hz), 6.94 (1H, d, J=7.9 Hz), 7.2 (1H, dd, J=6.9 Hz, 7.9 Hz), 7.35 (1H, d, J=6.9 Hz), 7.51 (1H, s), 8.30 (1H, s), 8.90 (1H, s), 10.74(1H, t, J=5.4 Hz), 11.97(1H, s).

### EXAMPLE 9

3-Ethyl-8-[2-(2-hydroxyethylamino)benzylamino]-2,3-dihydro-1H-imidazo[4,5-g]quinazoline-2-one 2 Hydrochloride (Compound 9)

The compound (0.91 g, 2.38 millimole) obtained in Reference Example 36 is suspended with the mixture solvent of methanol (90 ml) and tetrahydrofuran (72 ml), and 10%-palladium carbon (50% hydration, 0.50 g) is added. Then, the solution is stirred for one night under a hydrogen gas environment. After the reaction is completed, the catalyst is separated through filtration by using filter aid, and the obtained filtrate is concentrated under reduced pressure. The obtained concentrated residuum is dissolved with N,N-dimethylacetoamide (37 ml). Then, urea (1.21 g, 20.1 millimole) is added, and the solution is stirred for 4 hours at 120 degrees Celsius, and then for 6 hours at 160 degrees Celsius. After the reaction is completed, the solvent is removed under reduced pressure. The obtained residuum is purified with silica gel column chromatography (eluted with chloroform/methanol=20), and the free base of the subject compound (0.45 g) is obtained. The obtained free base is suspended with methanol (10 ml), and a sufficient amount of 4N hydrochloric acid-ethyl acetate is added under ice-chilled conditions. The solution is concentrated to half under reduced pressure, the precipitated crystal is washed with ether-methanol, and the subject compound (0.28 g, 26%) is obtained.

¹H-NMR (DMSO-d₆,) δ (ppm): 1.27 (3H, t, J=7.2 Hz), 3.37-3.42 (2H, m), 3.74-3.78 (2H, m), 3.93 (2H, q, J=7.2 Hz), 4.91 (2H, d, J=5.0 Hz), 4.98 (1H, br), 6.30-6.70 (1H, br), 7.05 (1H, dd, J=6.9 Hz, 7.3 Hz), 7.18(1H, d, J=7.6 Hz), 7.31 (1H, dd, J=7.3 Hz, 7.6 Hz), 7.44 (1H, d, J=6.9 Hz), 7.53 (1H, s), 8, 25(1H, s), 8.85 (1H, s), 10.83 (1H.br), 12.04 (1H, s).

### EXAMPLE 10

3-Ethyl-8-[2-(2-methoxyethylamino)benzylamino]-2,3-dihydro-1H-imidazo[4,5-g]quinazoline-2-one 2 Hydrochloride (Compound 10)

Using the compound obtained in Reference Example 38 as source material, the subject compound is obtained by using the same method as described in Example 1.

¹H-NMR (DMSO-d₆) δ (ppm): 1.28 (3H, t, J=7.2 Hz), 3.27 (3H, s), 3.42-3.49 (2H, m), 3.63 (2H, t, J=5.4 Hz), 3.94 (2H, q, J=7.2 Hz), 4.88 (2H, d, J=5.4 Hz), 6.00-6.40 (1H, m), 6.93 (1H, dd, J=7.4 Hz, 7.4 Hz), 7.03 (1H, d, J=7.9 Hz), 7.26 (1H, dd, J=7.4 Hz, 7.9 Hz), 7.38 (1H, d, J=7.4 Hz), 7.54 (1H, s), 8.2 3 (1H, s), 8.82(1H, s), 10.68 (1H, br), 12.00 (1H, s).

### EXAMPLE 11

3-Ethyl-8-(2-morpholinobenzylamino)-2,3-dihydro-1H-imidazo[4,5-g]quinazoline-2-one 1 Hydrochloride (Compound 11)

The 7-ethylamino-4-(2-morpholinobenzylamino)-6-nitroquinazoline (3.10 g, 7.60 millimole) obtained in Reference Example 39 is suspended with the mixture solvent of methanol (100 ml) and tetrahydrofuran (150 ml). 10%-Palladium carbon catalyst (0.31 g) is added to this suspension, and the suspension is stirred for 8 hours at room temperature under a hydrogen gas environment. After the reaction is completed, the catalyst is separated through filtration by using filter aid, and the obtained filtrate is divided into halves. One of the halves of the filtrate is concentrated under reduced pressure. The obtained concentrated residuum is dissolved with acetonitrile (100 ml). Then, N,N′-carbonyldiimidazole (1.80 g, 11.1 millimole) is added, and the solution is stirred for 6 hours while the solvent is refluxed. After the reaction is completed, the solvent is removed under reduced pressure. The obtained solid is separated through filtration, and washed with water and methanol. In addition, it is dried, and the free base of the subject compound (1.27 g) is obtained. The obtained free base is suspended with methanol (50 ml), and a sufficient amount of 4N hydrochloric acid-ethyl acetate is added under ice-chilled conditions. The solution is concentrated to half under reduced pressure, precipitated crystal is washed with ether-ethanol, and the subject compound (1.50 g, 89%) is obtained.

¹H-NMR (DMSO-d₆) δ (ppm): 1.28 (3H, t, J=7.1 Hz), 2.89-2.92 (4H, m), 3.73-3.77 (4H, m), 3.96 (2H, q, J=7.1 Hz), 5.05 (2H, d, J=5.3 Hz), 7.15 (1H, dd, J=7.3 Hz, 7.9 Hz), 7.31-7.43 (3H, m), 7.58 (1H, s), 8.26 (1H, s), 8.91 (1H, s), 10.37 (1H, br), 12.06(1H, s).

### EXAMPLE 12

3-Ethyl-8-[2-(4-hydroxymethylpiperidino)benzylamino]-2,3-dihydro-1H-imidazo[4,5-g]quinazoline-2-one 2 Hydrochloride (Compound 12)

Using the compound obtained in Reference Example 40, the subject compound is obtained by using the same method as described in Example 9.

¹H-NMR (DMSO-d₆) δ (ppm): 1.30 (3H, t, J=7.2 Hz), 1.70-2.05 (5H, m), 3.20-3.60 (6H, m), 3.94 (2H, q, J=7.2 Hz), 4.90 (1H, br), 5.12 (2H, s), 7.27-7.60 (4H, m), 7.56 (1H, s), 8.21 (1H, s), 8.91 (1H, s), 11.70 (1H, br), 11.98 (1H, s).

### EXAMPLE 13

3-Eethyl-8-(2-piperidinobenzylamino)-2,3-dihydro-1H-imidazo[4,5-g]quinazoline-2-one 2 Hydrochloride (Compound 13)

Using the compound obtained in Reference Example 41, the subject compound is obtained by using the same method as described in Example 11.

¹H-NMR (DMSO-d₆) δ (ppm) 1.28 (3H, t, J=7.1 Hz), 1.62-1.75 (2H, m), 1.80-1.98 (4H, m), 4.00 (2H, q, J=7.1 Hz), 4.20-4.43 (4H, m), 5.08 (2H, d, J=5.3 Hz), 7.23-7.34 (1H, m), 7.36-7.58 (3H, m), 7.62 (1H, s), 8.28 (1H, s), 8.92 (1H, s), 10.60 (1H, br), 12.08 (1H, s).

### EXAMPLE 14

3-Eethyl-8-[2-(4-methyl-1-piperazinyl)benzylamino]-2,3-dihydro-1H-imidazo[4,5-g]quinazoline-2-one 3 Hydrochloride (Compound 14)

Using the compound obtained in Reference Example 42, the subject compound is obtained by using the same method as described in Example 11.

¹H-NMR (DMSO-d₆) δ (ppm): 1.28 (3H, t, J=7.1 Hz), 2.85 (3H, s), 3.23-3.44 (6H, m), 3.50-3.54 (2H, m), 3.95 (2H, q, J=7.1 Hz), 5.03 (2H, d, J=5.3 Hz), 7.10 (1H, dd, J=6.6 Hz, 6.9 Hz), 7.22 (1H, d, J=7.6 Hz), 7.27-7.33 (2H, m), 7.57(1H, s), 8.27 (1H, s), 8.79 (1H, s), 10.51 (1H, t, J=5.3 Hz), 12.04 (1H, s).

### EXAMPLE 15

3-Ethyl-8-(2-thiomorpholinobenzylamino)-2,3-dihydro-1H-imidazo[4,5-g]quinazoline-2-one 1 Hydrochloride (Compound 15)

Using the compound obtained in Reference Example 43, the subject compound is obtained by using the same method as described in Example 11.

¹H-NMR (DMSO-d₆) δ (ppm): 1.30 (3H, t, J=7.2 Hz), 2.80-2.95 (4H, m), 3.15-3.28 (4H, m), 3.93 (2H, q, J=7.2 Hz), 5.02 (2H, d, J=5.4 Hz), 7.07(1H, dd, J=6.9 Hz), 7.4 Hz), 7.21-7.32 (3H, m).7.56 (1H, s), 8.21 (1H, s), 8.77 (1H, s), 10.36 (1H, t, J=5.4 Hz), 11.97 (1H, s).

### EXAMPLE 16

3-Ethyl-8-(3-morpholinobenzylamino)-2,3-dihydro-1H-imidazo[4,5-g]quinazoline-2-one 2 Hydrochloride (Compound 16)

Using the compound obtained in Reference Example 44, the subject compound is obtained by using the same method as described in Example 11.

¹H-NMR (DMSO-d₆) δ (ppm): 1.29 (3H, t, J=7.2 Hz), 3.23-3.31 (4H, m), 3.85-4.00 (6H, m), 4.93 (2H, d, J=5.4 Hz), 711 (1H, d, J=7.4 Hz), 7.23 (1H, d, J=7.4 Hz), 7.32 (1H, dd, J=7.4 Hz, 7.4 Hz), 7.42 (1H, s), 7.53 (1H, s), 8.21 (1H, s), 8.79 (1H, s), 10.57 (1H, t, J=5.4 Hz), 11.98 (1H, s).

### EXAMPLE 17

3-Ethyl-8-(3-piperidinobenzylamino)-2,3-dihydro-1H-imidazo[4,5-g]quinazoline-2-one 2 Hydrochloride (Compound 17)

Using the compound obtained in Reference Example 45, the subject compound is obtained by using the same method as described in Example 11.

¹H-NMR (DMSO-d₆) δ (ppm): 1.29 (3H, t, J=7.1 Hz), 1.66-1.70 (2H, m), 1.80-2.11 (4H, m), 3.35-3.49 (4H, m), 3.96 (2H, q, J=7.1 Hz), 4.96 (2H, d, J=5.3 Hz), 7.45-7.55 (3H, m), 7.66-7.75 (1H, m), 7.87-7.90 (1H, m), 8.18 (1H, s), 8.82 (1H, s), 10.57 (1H, br), 12.00 (1H, s).

### EXAMPLE 18

3-Ethyl-8-[3-(4-methyl-1-piperazinyl)benzylamino]-2,3-dihydro-1H-imidazo[4,5-g] quinazoline-2-one 3 Hydrochloride (Compound 18)

Using the compound obtained in Reference Example 46, the subject compound is obtained by using the same method as described in Example 11.

¹H-NMR (DMSO-d₆) δ (ppm): 1.30 (3H, t, J=7.1 Hz), 2.82 (3H, 3.15-3.23 (4H, m), 3.43-3.51 (2H, m), 3.74-3.82 (2H, m), 3.93 (2H, J=7.1 Hz), 4.90 (2H, d, J=5.6 Hz), 6.88-6.95 (2H, m), 7.10 (1H, s), 7.23 (1H, dd, J=7.9 Hz, 7.9 Hz), 7.54 (1H, s), 8.24 (1H, s), 8.82 (1H, s), 10.57 (1H, t, J=5.6 Hz), 12.00 (1H, s).

### EXAMPLE 19

3-Ethyl-8-(4-piperidinobenzylamino)-2,3-dihydro-1H-imidazo[4,5-g]quinazoline-2-one 2 Hydrochloride (Compound 19)

Using the compound obtained in Reference Example 48, the subject compound is obtained by using the same method as described in Example 11.

¹H-NMR (DMSO-d₆) δ (ppm): 1.29 (3H, t, J=6.9 Hz), 1.60-1.79 (2H.m), 1.81-2.11(4H, m), 3.35-3.49 (4H, m), 3.96 (2H, q, J=6.9 Hz), 4.95 (2H, d, J=5, 6 Hz), 7.49 (1H, s), 7.50-7.55 (2H, m), 7.72-7.77 (2H, m), 8.18 (1H, s), 8.81 (1H, s), 10.58 (1H, br), 12.00 (1H, s).

### EXAMPLE 20

3-Ethyl-8-[4-(4-methyl-1-piperazinyl)benzylamino]-2,3-dihydro-1H-imidazo[4,5-g]quinazoline-2-one 3 Hydrochloride (Compound 20)

Using the compound obtained in Reference Example 49, the subject compound is obtained by using the same method as described in Example 11.

¹H-NMR (DMSO-d₆) δ (ppm): 1.28 (3H, t, J=7.1 Hz), 2.80 (3H, s), 3.06-3.21 (411,m), 3.41-3.50 (2H, m), 3.75-3.85 (2H, m), 3.94 (2H, q, J=7.1 Hz), 4.84 (2H, d, J=5.6 Hz), 6.97 (2H, d, J=8.9 Hz), 7.31 (2H, d, J=8.9 Hz), 7.53 (1H, s), 8.20 (1H, s), 8.90 (1H, s), 10.57 (1H, t, J=5.6 Hz), 12.00 (1H, s).

### EXAMPLE 21

3-Ethyl-8-(4-thiomorpholinobenzylamino)-2,3-dihydro-1H-imidazo[4,5-g]quinazoline-2-one 2 Hydrochloride (Compound 21)

Using the compound obtained in Reference Example 51, the subject compound is obtained by using the same method as described in Example 11.

¹H-NMR (DMSO-d₆) δ (ppm): 1.29 (3H, t, J=7.2 Hz), 2.90-3.10(4H, m), 3.55-3.70 (4H, m), 3.95 (2H, q, J=7.2 Hz), 4.90 (2H, d, J=5.5 Hz), 7.35-7.47 (4H, m), 7.50 (1H, s), 8.19 (1H, s), 8.79 (1H, s), 10.55(1H, t, J=5.5 Hz), 11.98 (1H, s).

### EXAMPLE 22

3-Ethyl-8-(2-methylaminobenzylamino)-2,3-dihydro-1H-imidazo[4,5-g]quinazoline-2-thione 2 Hydrochloride (Compound 22)

7-Ethylamino-4-(2-methylaminobenzylamino)-6-nitroquinazoline (4.00 g, 11.4 millimole) obtained in Reference Example 28 is suspended with the mixture solvent of methanol (100 ml) and tetrahydrofuran (150 ml). 10%-Palladium carbon catalyst (0.40 g) is added to this suspension, and the suspension is stirred for one night at room temperature under a hydrogen gas environment. After the reaction is completed, the catalyst is separated through filtration by using filter aid, and the obtained filtrate is divided into halves. Triethylamine (1.60 ml, 11.5 millimole) and carbon disulfide (5.10 ml, 84.7 millimole) is added to one of these halves solution (not concentrated), and the solution is stirred for one night at room temperature. After the reaction is completed, the solvent is removed under reduced pressure, and ether is added to the obtained residuum. Precipitated crystal is separated through filtration, dried, and the free base of the subject compound (2.40 g) is obtained. The obtained free base is suspended with methanol (80 ml), and a sufficient amount of 4N hydrochloric acid-ethyl acetate is added under ice-chilled conditions. The solution is concentrated to half under reduced pressure, the precipitated crystal is washed with ether-ethanol, and the subject compound (2.15 g, 86%) is obtained.

¹H-NMR (DMSO-d₆) δ (ppm): 1.32 (3H, t, J=7.2 Hz), 2.91 (3H, s), 4.10 (1H, br), 4.36 (2H, q, J=7.2 Hz), 4.92 (2H, br), 6.91-7.02 (2H, m), 7.25-7.33 (2H, m), 7.7 2 (1H, s) 8.43 (1H, s), 8.89 (1H, s), 10.77 (1H, br), 13.73 (1H, s).

### EXAMPLE 23

3-Ethyl-8-(4-methylaminobenzylamino)-2,3-dihydro-1H-imidazo[4,5-g]quinazoline-2-thione 2 Hydrochloride (Compound 23)

The compound (5.40 g, 15.3 millimole) obtained in Reference Example 29 is suspended with the mixture solvent of methanol (700 ml) and tetrahydrofuran (900 ml). 10%-Palladium carbon catalyst (0.81 g) is added to this suspension, and the suspension is stirred for 3.5 hours at room temperature under a hydrogen gas environment. After the reaction is completed, the catalyst is separated through filtration by using filter aid. Triethylamine (13.6 ml, 97.7 millimole) and carbon disulfide (51.0 ml, 847 millimole) were added to ¾ of the obtained filtrate, and the solution is stirred for one night at room temperature. In order to complete the reaction, carbon disulfide (50.0 ml, 830 millimole) is added, and the solution is stirred for one night at room temperature. After the reaction is completed, the solvent is removed under reduced pressure. The obtained residuum is purified with silica gel column chromatography (eluted with chloroform/methanol=80), and the free base of the subject compound (1.05 g) is obtained. The obtained free base is suspended with methanol (100 ml), and a sufficient amount of 4N hydrochloric acid-ethyl acetate is added under ice-chilled conditions. The solution is concentrated to half under reduced pressure, the precipitated crystal is washed with ether-ethanol, and the subject compound (9.780 g, 16%) is obtained.

¹H-NMR (DMSO-d₆) δ (ppm): 1.30 (3H, t, J=6.9 Hz), 2.82 (3H, s), 3.60 (1H, br), 4.35 (2H, q, J=6.9 Hz), 4.93 (2H, d, J=5.6 Hz), 7.32 (2H, d, J=8.3 Hz), 7.49 (2H, d, J=8.3 Hz), 7.75 (1H, s), 8.41 (1H, s), 8.86 (1H, s), 10.83(1H, J=5.6 Hz), 13.79(1H, s).

### EXAMPLE 24

8-(4-Benzylaminobenzylamino)-3-ethyl-2,3-dihydro-1H-imidazo[4,5-g]quinazoline-2-thione 2 Hydrochloride (Compound 24)

Using the compound obtained in Reference Example 30, the subject compound is obtained by using the same method as described in Example 22.

¹H-NMR (DMSO-d₆) δ (ppm): 1.32 (3H, t, J=7.2 Hz), 4.31-4.37 (4H, m), 4.60 (1H, br), 4.86 (2H, d, J=5.4 Hz), 6.90-7.02 (2H, m), 7.20-7.43 (7H, m), 7.70 (1H, s), 8.36 (1H, s), 8.83 (1H, s), 10.83 (1H, t, J=5.4 Hz), 13.70 (1H, s).

### EXAMPLE 25

3-Ethyl-8-(4-isopropylaminobenzylamino)-2,3-dihydro-1H-imidazo[4,5-g]quinazoline-2-thione 2 Hydrochloride (Compound 25)

Using the compound obtained in Reference Example 31, the subject compound is obtained by using the same method as described in Example 22.

¹H-NMR (DMSO-d₆) δ (ppm): 1.24-1.34 (9H, m), 3.52-3.68 (2H, m), 4.36 (2H, q, J=7.3 Hz), 4.97 (2H, d, J=5.3 Hz), 7.43-7.54 (4H, m), 7.73 (1H, s), 8.39(1H, s), 8.86 (1H, s), 10.78 (1H, t, J=5.3 Hz), 13.75 (1H, s).

### EXAMPLE 26

3-Eethyl-8-(4-propylaminobenzylamino)-2,3-dihydro-1H-imidazo[4,5-g]quinazoline-2-thione 2 Hydrochloride (Compound 26)

Using the compound obtained in Reference Example 32, the subject compound is obtained by using the same method as described in Example 22.

¹H-NMR (DMSO-d₆) δ (ppm): 0.93 (3H, t, J=7.3 Hz), 1.32 (3H, t, J=6.9 Hz), 1.60-1.70 (2H, m), 3.12 (2H, t, J=7.4 Hz), 3.80 (1H, br), 4.36 (2H, q, J=6.9 Hz), 4.92 (2H, d, J=5.3 Hz), 7.21-7.24 (2H, m), 7.40-7.44 (2H, m), 7.68 (1H, s), 8.36 (1H, s), 8.87 (1H, s), 10.68 (1H, br), 13.76 (1H, s).

FAB-MASS: m/z calculated for C₂₁H₂₄N₅S 392, observed 393 (M+1).

### EXAMPLE 27

3-Ethyl-8-(4-ethylaminobenzylamino)-2,3-dihydro-1H-imidazo[4,5-g]quinazoline-2-thione 2 Hydrochloride (Compound 27)

Using the compound obtained in Reference Example 33, the subject compound is obtained by using the same method as described in Example 22.

¹H-NMR (DMSO-d₆) δ (ppm): 1.22-1.35 (6H, m), 3.25 (2H, q, J=7.3 Hz), 3.60 (1H, br), 4.36 (2H, q, J=7.3 Hz), 4.95 (2H, d, J=5.3 Hz), 7.37-7.40 (2H, m), 7.47-7.51 (2H, m), 7.70 (1H, s), 8.38 (1H, s), 8.86 (1H, s), 10.74 (1H, t, J=5.3 Hz), 13.74(1H, s).

### EXAMPLE 28

3-Ethyl-8-[2-(2-morpholinoethylamino)benzylamino]-2,3-dihydro-1H-imidazo[4,5-g]quinazoline-2-thione 3 Hydrochloride (Compound 28)

Using the compound obtained in Reference Example 34, the subject compound is obtained by using the same method as described in Example 22.

¹H-NMR (DMSO-d₆) δ (ppm): 1.32 (3H, t, J=7.2 Hz), 3.10-3.30 (2H, m), 3.35-3.60 (6H, m), 3.85-4.00 (4H, m), 4.35 (2H, q, J=7.2 Hz), 4.60 (1H, br), 4.86 (2H, d, J=4.9 Hz), 6.64 (1H, dd, J=7.4 Hz, 7.9 Hz), 6.76 (1H, d, J=7.9 Hz), 7.15(1H, dd, J=7.4 Hz, 8.4 Hz), 7.24 (1H, d, J=8.4 Hz), 7.77 (1H, s), 8.53 (1H, s), 8.89 (1H, s), 10.85 (1H, t, J=4.9 Hz), 13.75(1H, s).

### EXAMPLE 29

3-Ethyl-8-[2-(3-morpholinopropylamino)benzylamino]-2,3-dihydro-1H-imidazo[4,5-g]quinazoline-2-thione 3 Hydrochloride (Compound 29)

Using the compound obtained in Reference Example 35, the subject compound is obtained by using the same method as described in Example 22.

¹H-NMR (DMSO-d₆) δ (ppm): 1.33 (3H, t, J=7.1 Hz), 2.20-2.24 (211, m), 3.00-3.15 (2H, m), 3.25-3.48 (6H, m), 3.90-3.97 (4H, m), 4.35 (2H, q, J=7.1 Hz), 4.88 (2H, d, J=4.6 Hz), 5.00 (1H, br), 6.73 (1H, dd, J=7.3 Hz, 7.6 Hz), 6.82 (1H, d, J=7.9 Hz), 7.18 (1H, dd, J=7.6 Hz, 7.9 Hz), 7.30 (1H, d, J=7.3 Hz), 7.74 (1H, s), 8.51 (1H, s), 8.96 (1H, s), 10.89 (1H, t, J=4.6 Hz), 13.72 (1H, s).

### EXAMPLE 30

3-Ethyl-8-[2-(2-hydroxyethylamino)benzylamino]-2,3-dihydro-1H-imidazo[4,5-g]quinazoline-2-thione 2 Hydrochloride (Compound 30)

Using the compound obtained in Reference Example 36, the subject compound is obtained by using the same method as described in Example 22.

¹H-NMR (DMSO-d₆) δ (ppm): 1.32 (3H, t, J=6.9 Hz), 3.30-3.42 (2H, m), 3.71-3.77 (2H, m), 4.35 (2H, q, J=6.9 Hz), 4.91 (2H, br), 6.50 (1H, br), 6.70 (1H, br), 6.85-7.02 (2H, m), 7.23-7.38 (2H, m), 7.71 (1H, s), 8.39 (1H, s), 8.90 (1H, s), 10.75 (1H, br), 13.73 (1H, s).

### EXAMPLE 31

3-Eethyl-8-[4-(2-hydroxyethylamino)benzylamino]-2,3-dihydro-1H-imidazo[4,5-g]quinazoline-2-thione 2 Hydrochloride (Compound 31)

Using the compound obtained in Reference Example 37, the subject compound is obtained by using the same method as described in Example 22.

¹H-NMR (DMSO-d₆) δ (ppm): 1.32 (3H, t, J=6.9 Hz), 3.25-3.28 (2H, m), 3.66 (2H, t, J=5.9 Hz), 4.36 (2H, q, J=6.9 Hz), 4.94 (2H, d, J=5.4 Hz), 5.10 (1H, br), 6.70 (1H, br), 7.34 (2H, d, J=8.4 Hz), 7.47 (2H, d, J=8.4 Hz), 7.73 (1H, s), 8.40 (1H, s), 8.84 (1H, s), 10.77 (1H, t, J=5.4 Hz), 13.72 (1H, s).

### EXAMPLE 32

3-Ethyl-8-[2-(2-methoxyethylamino)benzylamino]-2,3-dihydro-1H-imidazo[4,5-g]quinazoline-2-thione 2 Hydrochloride (Compound 32)

Using the compound obtained in Reference Example 38, the subject compound is obtained by using the same method as described in Example 22.

¹H-NMR (DMSO-d₆) δ (ppm): 1.32 (3H, J=7.1 Hz), 3.25 (3H, s), 3.38-3.47 (2H, m), 3.60 (2H, t, J=5.3 Hz), 4.35 (2H, q, J=7.1 Hz), 4.90 (2H, d, J=4.6 Hz), 5.80 (1H, br), 6.84-6.97 (2H, m), 7.22-7.24 (2H, m), 7.75 (1H, s), 8.41 (1H, s), 8.88 (1H, s), 10.82 (1H, br), 13.20 (1H, s).

### EXAMPLE 33

3-Ethyl-8-(2-morpholinobenzylamino)-2,3-dihydro-1H-imidazo[4,5-g]quinazoline-2-thione 1 Hydrochloride (Compound 33)

The 7-ethylamino-4-(2-morpholinobenzylamino)-6-nitroquinazoline (2.00 g, 4.90 millimole) obtained in Reference Example 39 is suspended with the mixture solvent of methanol (100 ml) and tetrahydrofuran (150 ml). 10%-Palladium carbon catalyst (0.20 g) is added to this suspension, and the suspension is stirred for one night at room temperature under a hydrogen gas environment. After the reaction is completed, the catalyst is separated through filtration by using filter aid. Triethylamine (1.40 ml, 10.1 millimole) and carbon disulfide (3.00 ml, 49.9 millimole) were added to the obtained filtrate, and the solution is stirred for one night at room temperature. In addition, in order to complete the reaction, the solution is stirred for 2 hours at 50 degrees Celsius. After the reaction is completed, the solvent is removed under reduced pressure. Then, the precipitated solid is separated through filtration. This solid is re-crystallized (twice) with N,N-dimethylformamide-water. Furthermore, it is purified with silica gel column chromatography (eluted with chloroform/methanol=100), and the free base of the subject compound is obtained. The obtained free base is suspended with methanol (50 ml), and a sufficient amount of 4N hydrochloric acid-ethyl acetate is added under ice-chilled conditions. The solution is concentrated to half under reduced pressure, the precipitated crystal is separated through filtration, and the subject compound (1.66 g, 74%) is obtained.

¹H-NMR (DMSO-d₆) δ (ppm): 1.32 (3H, t, J=6.9 Hz), 2.91-2.94 (4H, m), 3.75-3.78 (4H, m), 4.36 (2H, q, J=6.9 Hz), 5.08 (2H, d, J=5.3 Hz), 7.08 (1H, dd, J=7.3 Hz, 8.9 Hz), 7.22-7.34 (3H, m), 7.77 (1H, s), 8.42 (1H, s), 8.85 (1H, s), 10.58 (1H, t, J=5.3 Hz), 13.72 (1H, s).

FAB-MASS: m/z calculated for C₂₂H₂₄N₆OS 420, observed 421 (M+1).

### EXAMPLE 34

3-Ethyl-8-[2-(4-hydroxymethylpiperidino)benzylamino]-2,3-dihydro-1H-imidazo[4,5-g]quinazoline-2-thione 2 Hydrochloride (Compound 34)

Using the compound obtained in Reference Example 40, the subject compound is obtained by using the same method as described in Example 33.

¹H-NMR (DMSO-d₆) δ (ppm): 1.33 (3H, t, J=6.9 Hz), 1.50-1.94 (5H, m), 3.20-3.50 (6H, m), 4.33 (2H, q, J=6.9 Hz), 4.80 (1H, br), 5.16 (2H, d, J=4.6 Hz), 7.16-7.60 (4H, m), 7.80 (1H, s), 8.43 (1H, s), 8.87 (1H, s), 10.89(1H, br), 13.71 (1H, s).

FAB-MASS: m/z calculated for C₂₄H₂₈N₆OS 448, observed 449 (M+1).

### EXAMPLE 35

3-Ethyl-8-(2-piperidinobenzylamino)-2,3-dihydro-1H-imidazo[4,5-g]quinazoline-2-thione 2 Hydrochloride (Compound 35)

Using the compound obtained in Reference Example 41, the subject compound is obtained by using the same method as described in Example 33.

¹H-NMR (DMSO-d₆) δ (ppm): 1.32 (3H, t, J=7.1 Hz), 1.64-1.91 (6H, m), 4.32-4.50 (6H, m), 5.12 (2H, d, J=4.3 Hz), 7.10-7.35 (1H, m), 7.40-7.60 (3H, m), 7.77 (1H, s), 8.41 (1H, s), 8.88 (1H, s), 10.76 (1H, br), 13.73 (1H, s).

FAB-MASS: m/z calculated for C₂₃H₂₆N₆S 418, observed 419(M+1).

### EXAMPLE 36

3-Ethyl-8-[2-(4-methyl-1-piperazinyl)benzylamino]-2,3-dihydro-1H-imidazo[4,5-g]quinazoline-2-thione 3 Hydrochloride (Compound 36)

Using the compound obtained in Reference Example 42, the subject compound is obtained by using the same method as described in Example 33.

¹H-NMR (DMSO-d₆) δ (ppm): 1.32 (3H, t, J=7.1 Hz), 2.85 (3H, s), 3.23-3.30 (6H, m), 3.46 3.52 (2H, m), 4.36 (2H, q, J=7.1 Hz), 5.06 (2H, d, J=5.3 Hz), 7.11 (1H, dd, J=6.9 Hz, 7.9 Hz), 7.23 (1H, d, J=7.6 Hz), 7.29-7.35 (2H, m), 7.80(1H, s), 8.47 (1H, s), 8.85 (1H, s), 10.69 (1H, t, J=5.3 Hz), 13.75 (1H, s).

### EXAMPLE 37

3-Ethyl-8-(2-thiomorpholinobenzylamino)-2,3-dihydro-1H-imidazo[4,5-g]quinazoline-2-thione 1 Hydrochloride (Compound 37)

Using the compound obtained in Reference Example 43, the subject compound is obtained by using the same method as described in Example 33.

¹H-NMR (DMSO-d₆) δ (ppm): 1.33 (3H, t, J=6.9 Hz), 2.72-2.91 (4H, m), 3.15-3.20 (4H, m), 4.36 (2H, q, J=6.9 Hz), 5.04 (2H, d, J=5.4 Hz), 7.07 (1H, dd, J=7.4 Hz, 7.4 Hz), 7.21-7.33 (3H, m), 7.76 (1H, s), 8.42 (1H, s), 8.84 (1H, s), 10.55 (1H, t, J=5.4 Hz), 13.70 (1H, s).

### EXAMPLE 38

3-Ethyl-8-(3-morpholinobenzylamino)-2,3-dihydro-1H-imidazo[4,5-g]quinazoline-2-thione 2% Hydrochloride (Compound 38)

Using the compound obtained in Reference Example 44, the subject compound is obtained by using the same method as described in Example 33.

¹H-NMR (DMSO-d₆) δ (ppm): 1.33 (3H, t, J=7.2 Hz), 3.20-3.25 (4H, m), 3.80-3.90 (4H, m), 4.36 (2H, q, J=7.2 Hz), 4.94 (2H, d, J=5.4HZ), 7.01 (1H, d, J=7.4 Hz), 7.10 (1H, d, J=7.9 Hz), 7.22-7.31 (2H, m), 7.73 (1H, s), 8.40 (1H, s), 8.85 (1H, s), 10.70 (1H, t, J=5.4 Hz), 13.70 (1H, s).

FAB-MASS: m/z calculated for C₂₂H₂₄N₆OS 420, observed 421 (M+1).

### EXAMPLE 39

3-Ethyl-8-(3-piperidinobenzylamino)-2,3-dihydro-1H-imidazo[4,5-g]quinazoline-2-thione 2 Hydrochloride (Compound 39)

Using the compound obtained in Reference Example 45, the subject compound is obtained by using the same method as described in Compound 33.

¹H-NMR (DMSO-d₆) δ (ppm): 1.32 (3H, t, J=7.1 Hz), 1.66-2.13 (6H, m) 3.44-3.62 (4H, m), 4.36 (2H, q, J=7.1 Hz), 4.98 (2H, d, J=5.3 Hz), 7.36-7.50 (2H, m), 7.55-7.73 (2H, m), 7.78-7.90 (1H, m), 8.40 (1H, s), 8.88 (1H, s), 10.80 (1H, br), 13.75 (1H, s).

FAB-MASS: m/z calculated for C₂₃H₂₆N₆OS 418, observed 419 (M+1).

### EXAMPLE 40

3-Ethyl-8-[3-(4-methyl-1-piperazinyl)benzylamino]-2,3-dihydro-1H-imidazo[4,5-g]quinazoline-2-thione 3 Hydrochloride (Compound 40)

Using the compound obtained in Reference Example 46, the subject compound is obtained by using the same method as described in Example 33.

¹H-NMR (DMSO-d₆) δ (ppm):1.23 (3H, 1, J=7.1 Hz), 2.72 (3H, s), 3.06-3.13 (4H, m), 3.39-3.42 (2H, m), 3.68-3.71 (2H, m), 4.27 (2H, J=7.1 Hz), 4.83 (2H, d, J=5.3 Hz), 6.79-6.86 (2H, m), 7.00 (1H, s), 7.14 (1H, dd, J=7.9 Hz, 7.9 Hz), 7.65 (1H, s), 8.33 (1H.s), 8.77 (1H, s), 10.65 (1H, t, J=5.3 Hz), 13.65 (1H, s).

### EXAMPLE 41

3-Ethyl-8-(4-morpholinobenzylamino)-2,3-dihydro-1H-imidazo[4,5-g]quinazoline-2-thione 2 Hydrochloride (Compound 41)

Using the compound obtained in Reference Example 47, the subject compound is obtained by using the same method as described in Example 33.

¹H-NMR (DMSO-d₆) δ (ppm): 1.30 (3H, t, J=7.1 Hz), 3.14-3.16 (4H, m), 3.76-3.80 (4H, m), 4.34 (2H, q, J=7.1 Hz), 4.87 (2H, d, J=5.6 Hz), 7.07 (2H, d, J=8.4 Hz), 7.34 (2H, d, J=8.4 Hz), 7.72 (1H, s), 8.38 (1H, s), 8.86 (1H, s), 10.70 (1H, br), 13.76(1H, s).

### EXAMPLE 42

3-Ethyl-8-(4-piperidinobenzylamino)-2,3-dihydro-1H-imidazo[4,5-g]quinazoline-2-thione 2 Hydrochloride (Compound 42)

Using the compound obtained in Reference Example 48, the subject compound is obtained by using the same method as described in Example 33.

¹H-NMR (DMSO-d₆) δ (ppm): 1.31 (3H, t, J=7.1 Hz), 1.50-1.79 (2H, m), 1.80-2.10 (4H, m), 3.50-3.90(4H, m), 4.36(2H, q, J=7.1 Hz), 4.97(2H, d, J=5.3 Hz), 7.56(2H, d, J=7.9 Hz), 7.70-7.87 (3H, m), 8.41 (1H, s), 8.86 (1H, s), 10.83 (1H, br), 13.76 (1H, s).

### EXAMPLE 43

3-Ethyl-8-[4-(4-methyl-1-piperazinyl)benzylamino]-2,3-dihydro-1H-imidazo[4,5-g]quinazoline-2-thione 3 Hydrochloride (Compound 43)

Using the compound obtained in Reference Example 49, the subject compound is obtained by using the same method as described in Example 33.

¹H-NMR (DMSO-d₆) δ (ppm):1.31(3H, t, J=7.1 Hz), 2.80(3H, s), 3.06-3.21(4H, m), 3.41-3.49 (2H, m), 3.75-3.80 (2H, m), 4.33 (2H, q, J=7.1 Hz), 4.87 (2H, d, J=5.6 Hz), 6.97 (2H, d, J=8.6 Hz), 7.32 (2H, d, J=8.6 Hz), 7.74 (H1, s), 8.39 (1H, s), 8.86 (1H, s), 10.72 (1H, J=5.6 Hz), 13.75(1H, s).

### EXAMPLE 44

3-Ethyl-8-[4-(1-pyrrolidinyl)benzylamino]-2,3-dihydro-1H-imidazo[4,5-g]quinazoline-2-thione 2 Hydrochloride (Compound 44)

Using the compound obtained in Reference Example 50, the subject compound is obtained by using the same method as described in Example 33.

¹H-NMR (DMSO-d₆) δ (ppm): 1.31 (3H, t, J=7.1 Hz), 1.99-2.05 (4H, m), 3.20-3.30 (4H, m), 4.34 (2H, q, J=7.1 Hz), 4.85 (2H, d, J=5.6 Hz), 6.70-6.78 (2H, m), 7.29-7.39 (2H, m).7.73 (1H, s), 8.38 (1H, s), 8.85 (1H, s), 10.68 (1H, t, J=5.6 Hz), 13.72(1H, s).

### EXAMPLE 45

3-Ethyl-8-(4-thiomorpholinobenzylamino)-2,3-dihydro-1H-imidazo[4,5-g]quinazoline-2-thione 2 Hydrochloride (Compound 45)

Using the compound obtained in Reference Example 51 the subject compound is obtained by using the same method as described in Example 33.

¹H-NMR (DMSO-d₆) δ (ppm): 1.31 (3H, t, J=7.2 Hz), 2.85-3.05 (4H, m), 3.55-3.70 (4H, m), 4.35 (2H, q, J=7.2 Hz), 4.91 (2H, d, J=5.4 Hz), 7.30-7.52 (4H, m), 7.74 (1H, s), 8.39 (1H, s), 8.86 (1H, s), 10.75 (1H, t, J=5.4 Hz), 13.74 (1H.s).

### EXAMPLE 46

3-Ethyl-8-{2-[N-(2-hydroxyethyl)methylamino]benzylamino}-2,3-dihydro-1H-imidazo[4,5-g]quinazoline-2-thione 2 Hydrochloride (Compound 46)

Using the compound obtained in Reference Example 53, the subject compound is obtained by using the same method as described in Example 33.

¹H-NMR (DMSO-d₆) δ (ppm): 1.31 (3H, t, J=7.1 Hz), 3.25 (3H, s), 3.80-3.90 (4H, m), 4.20 (1H, br), 4.35 (2H, q, J=7.1 Hz), 5.13 (2H, br), 7.35-7.60 (3H, 7.75-7.80 (2H, m), 8.45 (1H, s), 8.99 (1H, s), 11.07 (1H, s), 13.70 (1H, s).

### EXAMPLE 47

3-Ethyl-8-[4-(3-hydroxymethylpiperidino)benzylamino]-2,3-dihydro-1H-imidazo[4,5-g]quinazoline-2-thione 2 Hydrochloride (Compound 47)

Using the compound obtained in Reference Example 55, the subject compound is obtained by using the same method as described in Example 33.

¹H-NMR (DMSO-d₆) δ (ppm): 1.31 (3H, t, J=7.1 Hz), 1.80-2.15 (3H, m), 2.20-2.60 (3H, m), 3.40-4.00 (6H, m), 4.34 (2H, q, J=7.1 Hz), 4.96 (2H, d, J=5.6 Hz), 7.53-7.79 (5H, m), 8.29 (1H, s), 8.87 (1H, s), 10.75 (1H, br), 13.70 (1H, s).

### EXAMPLE 48

3-Ethyl-8-(4-morpholinobenzylamino)-2,3-dihydro-1H-imidazo[4,5-g]quinazoline-2-one 2 Hydrochloride (Compound 48)

Using the compound obtained in Reference Example 47 as source material, the subject compound is obtained by using the same method as described in Example 1.

¹H-NMR (DMSO-d₆) δ (ppm): 1.26 (3H, t, J=6.9 Hz), 3.17-3.20 (4H, m), 3.84-3.97 (6H, m), 4.87 (2H, d, J=5.3 Hz), 7.24-7.27 (2H, m), 7.39-7.43 (2H, m), 7.53 (1H, s), 8.22 (1H, s), 8.79 (1H, s), 10.02 (1H, br), 12.03 (1H, s).

### EXAMPLE 49

3-Ethyl-8-[4-(2-hydroxyethylamino)benzylamino]-2,3-dihydro-1H-imidazo[4,5-g]quinazoline-2-one 2 Hydrochloride (Compound 49)

Using the compound obtained in Reference Example 37 as source material, the subject compound is obtained by using the same method as described in Example 9.

¹H-NMR (DMSO-d₆) δ (ppm): 1.26 (3H, t, J=7.1 Hz), 3.26-3.30 (2H, m), 3.64-3.68 (2H, m), 3.93 (2H, q, J=7.1 Hz), 4.07 (1H, br), 4.91 (2H, d, J=5.6 Hz), 6.50 (1H, br), 7.38 (2H, d, J=8.2 Hz), 7.49 (2H, d, J=8.2 Hz), 7.52 (1H, s), 8.23 (1H, s), 8.80 (1H, s), 10.70 (1H, br), 12.04 (1H, s).

### EXAMPLE 50

3-Ethyl-8-[2-(1-imidazolyl)benzylamino]-2,3-dihydro-1H-imidazo[4,5-g]quinazoline-2-one 3 Hydrochloride (Compound 50)

Using the compound obtained in Reference Example 57 as source material, the subject compound is obtained by using the same method as described in Example 1.

¹H-NMR (DMSO-d₆) δ (ppm): 1.26 (3H, t, J=7.3 Hz), 3.93 (2H, q, J=7.3 Hz), 4.74 (2H, d, J=5.3 Hz), 7.54-7.65 (4H, m), 7.80 (1H, d, J=7.6 Hz), 7.90 (1H, s), 8.12 (1H, s), 8.25 (1H, s), 8.71 (1H, s), 9.61 (1H, s), 10.86 (1H, t, J=5.3 Hz), 12.05 (1H, s).

### EXAMPLE 51

3-Ethyl-8-[2-(1-imidazolyl)benzylamino]-2,3-dihydro-1H-imidazo[4,5-g]quinazoline-2-thione 3 Hydrochloride (Compound 51)

Using the compound obtained in Reference Example 57 as source material, the subject compound is obtained by using the same method as described in Example 22.

¹H-NMR (DMSO-d₈) δ (ppm): 1.30 (3H, t, J=7.1 Hz), 4.34 (2H, q, J=7.1 Hz), 4.78 (2H, d, J=5.0 Hz), 7.53-7.67 (3H, m), 7.77-7.82 (2H, m), 7.90 (1H, s), 8.10 (1H, s)., 8.41(1H, s), 8.77 (1H, s), 9.60 (1H, s), 10.94 (1H, br), 13.78 (1H, s).

### EXAMPLE 52

8-[2-(1-Perhydroazocinyl)benzylamino]-3-ethyl-2,3-dihydro-1H-imidazo[4,5-g]quinazoline-2-one 2 Hydrochloride (Compound 52)

Using the compound obtained in Reference Example 59 as source material, the subject compound is obtained by using the same method as described in Example 1.

¹H-NMR (DMSO-d₆) δ (ppm): 1.28(3H, t, J=7.1 Hz), 1.60-1.90(10H, m), 3.10-3.30 (4H, m), 3.96(2H, q, J=7.1 Hz), 5.09(2H, d, J=5.3 Hz), 7.00-7.40(4H, m), 7.56(1H, s), 8.25 (1H, s), 8.79(1H, s), 10.54(1H, br), 12.03 (1H.s).

### EXAMPLE 53

8-[2-(1-Perhydroazocinyl)benzylamino]-3-ethyl-2,3-dihydro-1H-imidazo[4,5-g]quinazoline-2-thione 2Hydrochloride (Compound 53)

Using the compound obtained in Reference Example 59 as source material, the subject compound is obtained by using the same method as described in Example 22.

¹H-NMR (DMSO-d₆) δ (ppm): 1.31(3H, t, J=7.1 Hz), 1.60-1.90(10H, m), 3.15-3.30 (4H, m), 4.35 (2H, q, J=7.1 Hz), 5.13 (2H, d, J=5.3 Hz), 7.05-7.42 (4H, m), 7.82 (1H, s), 8.48 (1H, s), 8.85 (1H, s), 10.81 (1H, br), 13.80 (1H, s).

### EXAMPLE 54

3-Ethyl-8-(2-propylaminobenzylamino)-2,3-dihydro-1H-imidazo[4,5-g]quinazoline-2-one 2 Hydrochloride (Compound 54)

From the compound obtained in Reference Example 61, the subject compound is obtained by using the same method as described in Example 1.

¹H-NMR (DMSO-d₆) δ (ppm): 0.94 (3H, t, J=7.4 Hz), 1.25 (3H, t, J=7.1 Hz), 1.70-1.78 (2H, m), 3.20-3.26 (2H, m), 3.91 (2H, q, J=7.1 Hz), 4.80 (1H.br), 4.93 (2H, d, J=4.3 Hz), 6.95-7.05 (1H, m), 7.10-7.20 (1H, m), 7.29 (1H, dd, J=5.9 Hz, 7.3 Hz), 7.39 (1H, d, J=7.3 Hz), 7.53 (1H, s), 8.23 (1H, s), 8.82 (1H, s), 10.74 (1H, br), 12.01 (1H, s).

### EXAMPLE 55

3-Ethyl-8-(2-propylaminobenzylamino)-2,3-dihydro-1H-imidazo[4,5-g]quinazoline-2-thione 2 Hydrochloride (Compound 55)

From the compound obtained in Reference Example 61, the subject compound is obtained by using the same method as described in Example 22.

¹H-NMR (DMSO-d₆) δ (ppm): 0.96 (3H, t, J=7.4 Hz), 1.30 (3H, 1, J=7.1 Hz), 1.69-1.78 (2H, m), 3.20-3.26 (2H, m), 4.34 (2H, q, J=7.1 Hz), 4.50 (1H, br), 4.96 (2H, br), 6.90-7.00 (1H, m), 7.01-7.15 (1H, m), 7.26-7.38 (2H, m), 7.78 (1H, s), 8.45 (1H, s), 8.90 (1H, s), 10.88 (1H, b r), 13.78 (1H, s).

### EXAMPLE 56

3-Ethyl-8-(2-isopropylaminobenzylamino)-2,3-dihydro-1H-imidazo[4,5-g]quinazoline-2-thione 2 Hydrochloride (Compound 56)

From the compound obtained in Reference Example 63, the subject compound is obtained by using the same method as described in Example 22.

¹H-NMR (DMSO-d₆) δ (ppm): 1.27-1.35 (9H, m), 3.60-4.00 (2H, m), 4.35 (2H, q, J=7.1 Hz), 5.01 (2H, br), 7.10-7.22 (1H, m), 7.23-7.48 (3H.m), 7.77 (1H, s), 8.44 (1H, s), 8.91 (1H, s), 10.90 (1H, br), 13.81 (1H, s).

### EXAMPLE 57

3-Ethyl-8-[2-(3-hydroxymethylpiperidino)benzylamino]-2,3-dihydro-H-imidazo[4,5-g]quinazoline-2-one 2 Hydrochloride (Compound 57)

Using the compound obtained in Reference Example 65 as source material, the subject compound is obtained by using the same method as described in Example 9.

¹H-NMR (DMSO-d₆) δ (ppm): 1.27 (3H, t, J=6.9 Hz), 1.75-2.20 (3H, m), 2.80-3.20 (3H, m), 3.30-3.55 (4H, m), 3.85-4.20 (4H, m), 5.08 (2H, br), 7.17-7.50 (4H, m), 7.56 (1H, S), 8.22 (1H, s), 8.81 (1H.s), 10.60 (1H, br), 12.03 (1H, s).

### EXAMPLE 58

3-Ethyl-8-[3-(4-hydroxymethylpiperidino)benzylamino]-2,3-dihydro-1H-imidazo[4,5-g]quinazoline-2-thione 2 Hydrochloride (Compound 58)

Using the compound obtained in Reference Example 67 as source material, the subject compound is obtained by using the same method as described in Example 33.

¹H-NMR (DMSO-d₆) δ (ppm): 1.30 (3H, t, J=6.9 Hz), 1.70-2.00 (4H, m), 3.40-3.80 (8H, m), 4.35 (2H, q, J=6.9 Hz), 4.98 (2H, d, J=5.6 Hz), 7.46-7.49 (2H, m), 7.60-7.95 (3H, m), 8.43 (1H, s), 8.88 (1H, s), 10.87 (1H, br), 13.79 (1H, s).

### EXAMPLE 59

3-Ethyl-8-{2-[4-(2-hydroxyethyl)piperidino]benzylamino}-2,3-dihydro-1H-imidazo[4,5-g]quinazoline-2-thione 2 Hydrochloride (Compound 59)

Using the compound obtained in Reference Example 69 as source material, the subject compound is obtained by using approximately the same method as described in Example 22.

¹H-NMR (DMSO-d₆) δ (ppm): 1.31 (3H, t, J=6.9 Hz), 1.46-1.56 (2H, m), 1.60-2.00 (4H, m), 3.10-3.4 3 (5H, m), 3.45-3.52 (2H, m), 4.35 (2H, q, J=6.9), 4.50 (1H, br), 5.10 (2H, br), 7.20-7.52 (4H, m), 7.79 (1H, s), 8.42 (1H, s), 8.88 (1H, s), 10.70 (1H, br), 13.76 (1H, s).

### EXAMPLE 60

3-Ethyl-8-(2-isopropylaminobenzylamino)-2,3-dihydro-1H-imidazo[4,5-g]quinazoline-2-one 2 Hydrochloride (Compound 60)

Using the compound obtained in Reference Example 63 as source material, the subject compound is obtained by using approximately the same method as described in Example 1.

¹H-NMR (DMSO-d₆) δ (ppm): 1.25-1.33 (9H, m), 3.75-3.90 (1H, m), 3.95 (2H, q, J=7.2 Hz), 4.27 (1H, br), 4.97 (2H, br), 7.00-7.48 (4H, m), 7.49 (1H, s), 8.18 (1H, s), 8.86 (1H, s), 10.60 (1H, br), 12.00 (1H, s).

### EXAMPLE 61

3-Ethyl-8-(2-ethylaminobenzylamino)-2,3-dihydro-1H-imidazo[4,5-g]quinazoline-2-one 2 Hydrochloride (Compound 61)

Using the compound obtained in Reference Example 73 as source material, the subject compound is obtained by using approximately the same method as described in Example 1.

¹H-NMR (DMSO-d₆) δ (ppm): 1.25-1.37 (6H, m), 3.35-3.46 (2H, m), 3.95 (2H, q, J=7.3 Hz), 4.60 (1H, br), 4.98 (2H, br), 7.05-7.15 (1H, m), 7.15-7.37 (2H, m), 7.44 (1H, d, J=7.6 Hz), 7.52 (1H, s), 8.23 (1H, s), 8.85 (1H, s), 10.71 (1H, s), 12.01 (1H, s).

### EXAMPLE 62

3-Ethyl-8-(2-ethylaminobenzylamino)-2,3-dihydro-1H-imidazo[4,5-g]quinazoline-2-thione 2 Hydrochloride (Compound 62)

Using the compound obtained in Reference Example 73 as source material, the subject compound is obtained by using approximately the same method as described in Example 22.

¹H-NMR (DMSO-d₆) δ (ppm): 1.28-1.34 (6H, m), 3.18-3.31 (2H, m), 4.20 (1H, br), 4.36 (2H, q, J=7.0 Hz), 4.95 (2H, br), 6.85-7.20 (2H, m), 7.25-7.40 (2H, m), 7.72 (1H, s), 8.41 (1H, s), 8.90 (1H, s), 10.70 (1H, br), 13.76 (1H, s).

### EXAMPLE 63

3-Ethyl-8-[2-(3-hydroxymethylpiperidino)benzylamino]-2,3-dihydro-1H-imidazo[4,5-g]quinazoline-2-thione 2 Hydrochloride (Compound 63)

Using the compound obtained in Reference Example 65 as source material, the subject compound is obtained by using approximately the same method as described in Example 22.

¹H-NMR (DMSO-d₆) δ (ppm): 1.32 (3H, t, J=7.1 Hz), 1.72-2.15 (3H, m), 2.70-3.00 (2H, m), 3.15-3.46(4H, m), 4.10-4.50(5H.m), 5.11(2H, d, J=5.0 Hz), 7.10-7.50(4H, m), 7.79 (1H, s), 8.43 (1H, s), 8.87 (1H, s), 10.84 (1H.br), 13.75 (1H, s).

### EXAMPLE 64

8-(2-Cyclopentylaminobenzylamino)-3-ethyl-2,3-dihydro-1H-imidazo[4,5-g]quinazoline-2-thione 2 Hydrochloride (Compound 64)

Using the compound obtained in Reference Example 75 as source material, the subject compound is obtained by using approximately the same method as described in Example 22.

¹H-NMR (DMSO-d₆) δ (ppm): 1.31 (3H, t, J=7.1 Hz), 1.51-1.90 (6H, m), 1.90-2.05 (2H, m), 3.90-4.00 (1H, m), 4.36 (2H, q, J=7.1 Hz), 4.50 (1H, br), 4.98 (2H, br), 6.95-7.15 (2H, m), 7.28-7.35 (2H, m), 7.74 (1H, s), 8.41 (1H, s), 8.91 (1H.s), 10.77 (1H, S), 13.77 (1H, s).

### EXAMPLE 65

8-(2-Butylaminobenzylamino)-3-ethyl-2,3-dihydro-1H-imidazo[4,5-g]quinazoline-2-thione 2 Hydrochloride (Compound 65)

Using the compound obtained in Reference Example 77 as source material, the subject compound is obtained by using approximately the same method as described in Example 22.

¹H-NMR (DMSO-d₆) δ (ppm): 0.91 (3H, t, J=7.3 Hz), 1.28-1.42 (5H, m), 1.64-1.72 (2H, m), 3.21-3.27 (2H, m), 4.35 (2H, q, J=7.1 Hz), 4.50 (1H, br), 4.94 (2H, br), 6.90-7.05 (2H, m), 7.25-7.35 (2H, m), 7.74 (1H, s), 8.42 (1H, s), 8.90 (1H, s), 10.79 (1H, b r), 13.78 (1H, s).

### EXAMPLE 66

3-Ethyl-8-[2-(1-pyrrolidinyl)benzylamino]-2,3-dihydro-1H-imidazo[4,5-g]quinazoline-2-one 2 Hydrochloride (Compound 66)

Using the compound obtained in Reference Example 79 as source material, the subject compound is obtained by using approximately the same method as described in Example 1.

¹H-NMR (DMSO-d₆) δ (ppm): 1.28 (3H, t, J=7.1 Hz), 2.05-2.17 (4H, m), 3.45-3.60 (2H, m), 3.96 (2H, q, J=7.1 Hz), 3.96-4.00 (2H, m), 5.08 (2H, br), 7.07-7.45 (4H, m), 7.50 (1H, s), 8.19 (1H, s), 8.84 (1H, s), 10.60 (1H, br), 11.99 (1H, s).

### EXAMPLE 67

8-(2-Butylaminobenzylamino)-3-ethyl-2,3-dihydro-1H-imidazo[4,5-g]quinazoline-2-one 2 Hydrochloride (Compound 67)

Using the compound obtained in Reference Example 77 as source material, the subject compound is obtained by using approximately the same method as described in Example 1.

¹H-NMR (DMSO-d₆) δ (ppm): 0.92 (3H, t, J=7.4 Hz), 1.28 (3H, t, J=7.1 Hz), 1.33-1.47 (2H, m), 1.68-1.80 (2H, m), 3.26-3.33 (2H, m), 3.95 (2H, q, J=7.1 Hz), 4.60 (1H, b r), 4.97 (2H, d, J=4.3 Hz), 7.00-7.10 (1H, m), 7.12-7.20 (1H, m), 7.25-7.35 (1H, m), 7.42 (1H, d, J=7.3 Hz), 7.52 (1H, s), 8.22 (1H, s), 8.84 (1H, s), 10.68 (1H, t, J=4.3 Hz), 12.00(1H, s).

### EXAMPLE 68

8-(2-Cyclohexylaminobenzylamino)-3-ethyl-2,3-dihydro-1H-imidazo[4,5-g]quinazoline-2-thione 2 Hydrochloride (Compound 68)

Using the compound obtained in Reference Example 81 as source material, the subject compound is obtained by using approximately the same method as described in Example 22.

¹H-NMR (DMSO-d₆) δ (ppm): 1.24-1.46 (7H, m), 1.48-1.67 (2H, m), 1.70-1.8 (2H, m), 2.03-2.08 (2H, m), 3.44-3.52 (1H, m), 4.20 (1H, br), 4.36 (2H, q, J=7.3 Hz), 5.02 (2H, s), 7.11-7.33 (3H, m), 7.44-7.48 (1H, m), 7.74 (1H, s), 8.41(1H, s), 8.91(1H, s), 10.80 (1H, s), 13.75 (1H, s).

### EXAMPLE 69

8-(2-Cyclohexylaminobenzylamino)-3-ethyl-2,3-dihydro-1H-imidazo[4,5-g]quinazoline-2-one 2 Hydrochloride (Compound 69)

Using the compound obtained in Reference Example 81 as source material, the subject compound is obtained by using approximately the same method as described in Example 1.

¹H-NMR (DMSO-d₆) δ (ppm): 1.24-1.31 (6H, m), 1.54-1.63 (3H, m), 1.77-1.82 (2H, m), 2.06-2.09 (2H, m), 3.45-3.60 (1H, m), 3.95 (2H, q, J=6.9 Hz), 4.45 (1H, br), 5.02 (2H, d, J=4.6 Hz), 7.21-7.45 (3H, m), 7.51-7.54 (2H, m), 8.22 (1H, s), 8.85 (1H.s), 10.72 (1H, 1, J=4.6 Hz), 12.01 (1H, s).

### EXAMPLE 70

3-Ethyl-8-[2-(1-pyrrolidinyl)benzylamino]-2,3-dihydro-1H-imidazo[4,5-g]quinazoline-2-thione 2 Hydrochloride (Compound 70)

Using the compound obtained in Reference Example 79, the subject compound is obtained by using approximately the same method as described in Example 22.

¹H-NMR (DMSO-d₆) δ (ppm): 1.31 (3H, t, J=7.1 Hz), 2.05-2.25 (4H, m), 3.60-3.85 (4H, m), 4.36 (2H, q, J=7.1 Hz), 5.17 (2H, br), 7.27-7.45 (1H, m), 7.48-7.65 (3H, m), 7.76 (1H, s), 8.43 (1H, s), 8.90 (1H, s), 10.92 (1H, br), 13.86 (1H, s).

### EXAMPLE 71

3-Ethyl-8-[2-(4-ethyl-1-piperazinyl)benzylamino]-2,3-dihydro-1H-imidazo[4,5-g] quinazoline-2-one 3 Hydrochloride (Compound 71)

Using the compound obtained in Reference Example 83, the subject compound is obtained by using approximately the same method as described in Example 1.

¹H-NMR (DMSO-d₆) δ (ppm) 1.25-1.36 (6H, m), 3.18-3.46 (8H, m), 3.54-3.58 (2H, m), 3.95 (2H, q, J=7.2 Hz), 5.03 (2H, d, J=5.6 Hz), 7.10 (1H, dd, J=7.6 Hz, 8.2 Hz), 7.22 (1H, d, J=6.9 Hz), 7.28-7.34 (2H, m), 7.57 (1H, s), 8.26 (1H, s), 8.80 (1H, s), 10.51 (1H, t, J=5.6 Hz), 12.05 (1H, s).

### EXAMPLE 72

3-Ethyl-8-[2-(2-methylpropylamino)benzylamino]-2,3-dihydro-1H-imidazo[4,5-g]quinazoline-2-one 2 Hydrochloride (Compound 72)

Using the compound obtained in Reference Example 85, the subject compound is obtained by using approximately the same method as described in Example 1.

¹H-NMR (DMSO-d₆) δ (ppm): 0.99 (6H, d, J=6.9 Hz), 1.28 (3H, t, J=7.1 Hz), 2.04-2.10 (1H, m), 3.06-3.09 (2H, m), 3.94 (2H, q, J=7.1 Hz), 4.90 (2H, d, J=5.3 Hz), 6.40(1H, br), 6.88-6.92 (1H, m), 6.95-7.02 (1H, m), 7.24 (1H, dd, J=7.6 Hz, 7.6 Hz), 7.35 (1H, d, J=7.3 Hz), 7.54 (1H, s), 8.24 (1H, s), 8.84 (1H, s), 10.70 (1H, t, J=5.3 Hz), 12.02 (1H, s).

### EXAMPLE 73

8-[2-(1-Perhydroazevinyl)benzylamino]-3-ethyl-2,3-dihydro-1H-imidazo[4,5-g]quinazoline-2-thione 2 Hydrochloride (Compound 73)

Using the compound obtained in Reference Example 87, the subject compound is obtained by using approximately the same method as described in Example 22.

¹H-NMR (DMSO-d₆) δ (ppm): 1.32 (3H, t, J=7.1 Hz), 1.75-1.86 (4H, m), 1.90-2.15 (4H, m), 3.50-3.70 (2H, m), 4.35 (2H, q, J=7.1 Hz), 4.60-4.85 (2H, m), 5.16 (2H, br), 7.27-7.50 (4H, m), 7.80 (1H, s), 8.46 (1H, s), 8.87 (1H, s), 11.00 (1H, s), 13.76 (1H, s).

### EXAMPLE 74

8-(2-Cyclopentylaminobenzylamino)-3-ethyl-2,3-dihydro-1H-imidazo[4,5-g]quinazoline-2-one 2 Hydrochloride (Compound 74)

Using the compound obtained in Reference Example 75, the subject compound is obtained by approximately using the same method as described in Example 1.

¹H-NMR (DMSO-d₆) δ (ppm): 1.27 (3H, t, J=7.1 Hz), 1.65-1.72 (2H, m), 1.75-1.90 (4H, m), 1.93-2.00 (2H, m), 3.94 (2H, q, J=7.1 Hz), 3.95-4.00 (1H, m), 5.00 (2H, d, J=4.6 Hz), 5.20 (1H, br), 7.09-7.20 (1H, m), 7.27-7.40 (2H.m), 7.42-7.50 (1H, m), 7.54 (1H, s), 8.23 (1H, s), 8.85 (1H, s), 10.72 (1H, t, J=4.6 Hz), 12.01 (1H, s).

### EXAMPLE 75

3-Ethyl-8-[2-(1-tricyclo[3.3.1.1^{3.7}]decyl)aminobenzylamino]-2,3-dihydro-1H-imidazo[4,5-g]quinazoline-2-thione 2 Hydrochloride (Compound 75)

Using the compound obtained in Reference Example 89, the subject compound is obtained by using approximately the same method as described in Example 22.

¹H-NMR (DMSO-d₆) δ (ppm): 1.33 (3H, t, J=7.1 Hz), 1.66-1.74 (6H, m), 2.12-2.52 (9H, m), 3.06 (1H, br), 4.37 (2H, q, J=7.1 Hz), 5.20 (2H, d, J=5.0 Hz), 7.46-7.54 (3H, m), 7.65-7.69 (1H, m), 7.78 (1H, s), 8.41 (1H, s), 8.95 (1H, s), 10.94 (1H, t, J=5.0 Hz), 13.76 (1H, s).

### EXAMPLE 76

3-Ethyl-8-{2-[4-(2-hydroxyethyl)piperidino]benzylamino}-2,3-dihydro-1H-imidazo[4,5-g]quinazoline-2-one 2 Hydrochloride (Compound 76)

Using the compound obtained in Reference Example 69, the subject compound is obtained by using approximately the same method as described in Example 9.

¹H-NMR (DMSO-d₆) δ (ppm): 1.30 (3H, t, J=7.1 Hz), 1.46-1.72 (4H, m), 1.75-1.90 (2H, m), 2.70-3.40 (7H, m), 3.97 (2H, q, J=7.1 Hz), 5.08 (2H, br), 6.40 (1H, br), 7.10-7.40 (4H, m), 7.48 (1H, s), 8.24 (1H, s), 8.81 (1H, s), 10.30 (1H, br), 11.95 (1H, s).

### EXAMPLE 77

3-Ethyl-8-[2-(4-ethyl-1-piperazinyl)benzylamino]-2,3-dihydro-1H-imidazo[4,5-g]quinazoline-2-thione 3 Hydrochloride (Compound 77)

Using the compound obtained in Reference Example 83, the subject compound is obtained by using approximately the same method as described in Example 22.

¹H-NMR (DMSO-d₆) δ (ppm): 1.31-1.39 (6H, m), 3.19-3.32 (8H, m), 3.50-3.61 (2H, m), 4.37 (2H, q, J=7.3 Hz), 5.07 (2H, d, J=5.6 Hz), 7.12 (1H, dd, J=6.9 Hz, 7.9 Hz), 7.23-7.35 (3H, m), 7.71 (1H, s), 8.42 (1H, s), 8.85 (1H, s), 10.57 (1H, t, J=5.6 Hz), 13.71 (1H, s).

### EXAMPLE 78

3-Ethyl-8-[2-(2-methylpropylamino)benzylamino]-2,3-dihydro-1H-imidazo[4,5-g]quinazoline-2-thione 2 Hydrochloride (Compound 78)

Using the compound obtained in Reference Example 85, the subject compound is obtained by using approximately the same method as described in Example 22.

¹H-NMR (DMSO-d₆) δ (ppm): 0.96 (6H, d, J=6.6 Hz), 1.32 (3H, t, J=7.1 Hz), 1.97-2.02 (1H, m), 2.99-3.03 (2H, m), 4.36 (2H, q, J=7.1 Hz), 4.90 (2H, d, J=4.6 Hz), 5.00 (1H, br), 6.75-6.84 (2H, m), 7.16-7.28 (2H, m), 7.72 (1H, s), 8.40 (1H, s), 8.89 (1H, s), 10.69 (1H, t, J=4.6 Hz), 13.73 (1H, s).

### EXAMPLE 79

3-Ethyl-8-[2-(4-hydroxybutylamino)benzylamino]-2,3-dihydro-1H-imidazo[4,5-g]quinazoline-2-thione 2 Hydrochloride (Compound 79)

Using the compound obtained in Reference Example 91, the subject compound is obtained by using approximately the same method as described in Example 22.

¹H-NMR (DMSO-d₆) δ (ppm): 1.32 (3H, t, J=6.9 Hz), 1.50-1.60 (2H, M), 1.75-1.86 (2H, m), 3.29-3.32 (2H, m), 3.41-3.50 (2H, m), 4.36 (2H, q, J=6.9 Hz), 4.99 (2H, br), 5.00 (1H, br), 5.30 (1H, br), 7.06-7.29 (2H, m), 7.32 (1H, dd, J=6.9 Hz, 8.6 Hz), 7.41 (1H, d, J=7.3 Hz), 7.73 (1H, s), 8.41 (1H, s), 8.90 (1H, s), 1 0.82 (1H, s), 13.74 (1H, s).

### EXAMPLE 80

8-[2-(4-Hydroxymethylpiperidino)benzylamino]-3-propyl-2,3-dihydro-1H-imidazo[4,5-g]quinazoline-2-thione 2 Hydrochloride (Compound 80)

Using the compound obtained in Reference Example 93, the subject compound is obtained by using approximately the same method as described in Example 22.

¹H-NMR (DMSO-d₆) δ (ppm): 0.95 (3H, t, J=7.4 Hz), 1.50-2.00 (7H, m), 3.20-3.70 (6H, m), 4.28 (2H, t, J=7.1 Hz), 5.15 (2H, br), 5.70 (1H, br), 7.20-7.55 (4H, m), 7.82 (1H, s), 8.43 (1H, s), 8.88 (1H, s), 10.45 (1H, br), 13.76 (1H, s).

### EXAMPLE 81

8-[2-(1-Perhydroazevinyl)benzylamino]-3-ethyl-2,3-dihydro-1H-imidazo[4,5-g]quinazoline-2-one 2 Hydrochloride (Compound 81)

Using the compound obtained in Reference Example 87, the subject compound is obtained by using approximately the same method as described in Example 1.

¹H-NMR (DMSO-d₆) δ (ppm): 1.30 (3H, t, J=7.3 Hz), 1.70-1.90 (4H, m), 1.95-2.10 (4H, m), 3.50-3.70 (4H, m), 3.96 (2H, q, J=7.3 Hz), 5.14 (2H, br), 7.26-7.80 (5H, m), 8.22 (1H, s), 8.81 (1H, s), 10.60 (1H, s), 11.99 (1H, s).

### EXAMPLE 82

3-Ethyl-8-[2-(4-methyl-1-homopiperazinyl)benzylamino]-2,3-dihydro-1H-imidazo[4,5-g]quinazoline-2-thione 3 Hydrochloride (Compound 82)

From the compound obtained in Reference Example 95, the subject compound is obtained by using approximately the same method as described in Example 22.

¹H-NMR (DMSO-d₆) δ (ppm): 1.34 (3H, t, J=7.1 Hz), 2.09-2.15 (1H, m), 2.27-2.33 (1H, m), 2.88 (3H, s), 3.14-3.20 (2H, m), 3.29-3.46 (2H, m), 3.47-3.59(4H, m), 4.37 (2H, q, J=7.1 Hz), 5.09 (2H, d, J=5.6HZ), 7.02-7.09 (1H, m), 7.28-7.31 (3H, m), 7.74 (1H, s), 8.47 (1H, s), 8.85 (1H, s), 10.65 (1H, t, J=5.6 Hz), 13.7 3 (1H, s).

### EXAMPLE 83

3-Ethyl-8-{2-[2-(2-hydroxyethoxy)ethylamino]benzylamino}-2,3-dihydro-1H-imidazo[4,5-g]quinazoline-2-thione 2 Hydrochloride (Compound 83)

From the compound obtained in Reference Example 97, the subject compound is obtained by using approximately the same method as described in Example 22.

¹H-NMR (DMSO-d₆) δ (ppm): 1.33 (3H, t, J=7.3 Hz), 3.41-3.52 (6H, m), 3.72 (2H, t, J=5.3 Hz), 4.36 (2H, q, J=7.3 Hz), 4.91 (2H, d, J=5.3 Hz), 5.55 (2H, br), 6.86-7.00 (2H, m), 7.25 (1H, dd, J=7.3 Hz, 7.9 Hz), 7.35 (1H, d, J=7.9 Hz), 7.70 (1H, s), 8.39 (1H, s), 8.92 (1H, s), 10.72 (1H, t, J=5.3 Hz), 13.72 (1H, s).

### EXAMPLE 84

3-Ethyl-8-[2-(4-methyl-1-homopiperazinyl)benzylamino]-2,3-dihydro-1H-imidazo[4,5-g]quinazoline-2-one 3 Hydrochloride (Compound 84)

From the compound obtained in Reference Example 95, the subject compound is obtained by using approximately the same method as described in Example 1.

¹H-NMR (DMSO-d₆) δ m): 1.28 (3H, t, J=7.3 Hz), 2.12-2.17 (1H, m), 2.25-2.32 (1H, m), 2.84 (3H, s), 3.12-3.18 (1H, m), 3.31-3.61 (6H, m), 3.94 (2H, q, J=7.3 Hz), 5.05 (2H, d, J=5.6 Hz), 7.04-7.10 (1H, m), 7.27-7.33 (3H, m), 7.56 (1H, s), 8.28 (1H, s), 8.79 (1H, s), 10.51 (1H, t, J=5.6 Hz), 12.02 (1H, s).

### EXAMPLE 85

8-[2-(4-Hydroxymethylpiperidino)benzylamino]-3-methyl-2,3-dihydro-1H-imidazo[4,5-g]quinazoline-2-thione 2 Hydrochloride (Compound 85)

Using the compound obtained in Reference Example 99, the subject compound is obtained by using approximately the same method as described in Example 22.

¹H-NMR (DMSO-d₆) δ (ppm): 1.56-1.87 (5H, m), 3.00-3.50 (7H, m), 3.74 (3H, s), 5.10 (2H, br), 7.13-7.37 (4H, m), 7.69 (1H, s), 8.39 (1H, s), 8.87 (1H, s), 10.60 (1H, br), 13.72 (1H, s).

### EXAMPLE 86

3-Ethyl-8-[2-(2-furilmethylamino)benzylamino]-2,3-dihydro-1H-imidazo[4,5-g]quinazoline-2-thione 2 Hydrochloride (Compound 86)

From the compound obtained in Reference Example 101, the subject compound is obtained by using approximately the same method as described in Example 22.

¹H-NMR (DMSO-d₆) δ (ppm): 1.30 (3H, t, J=7.1 Hz), 4.30-4.46 (4H, m), 4.83 (2H, d, J=5.3 Hz), 6.03 (1H, br), 6.30-6.37 (2H, m), 6.66 (1H, dd, J=7.3 Hz, 8.6 Hz), 6.77 (1H, d, J=8.2 Hz), 7.12 (1H, d, J=8.2 Hz, 8.6 Hz), 7.20 (1H, d, J=7.3 Hz), 7.50 (1H, s), 7.78 (1H, s), 8.44 (1H, s), 8.81 (1H, s), 10.79 (1H, t, J=5.3 Hz), 13.81 (1H, s).

### EXAMPLE 87

3-Ethyl-8-[2-(1,2,3,4-tetrahydroisoquinoline-2-yl)benzylamino]-2,3-dihydro-1H-imidazo[4,5-g]quinazoline-2-thione 1 Hydrochloride (Compound 87)

From the compound obtained in Reference Example 103, the subject compound is obtained by using approximately the same method as described in Example 22.

¹H-NMR (DMSO-d₆) δ (ppm): 1.31 (3H, t, J=7.1 Hz), 2.95-3.03 (2H, m), 3.25-3.37 (2H.m), 4.18 (2H, br), 4.34 (2H, q, J=7.1 Hz), 5.10 (2H, d, J=5.0 Hz), 7.10-7.15 (5H, m), 7.32-7.40 (3H, m), 7.76 (1H, s), 8.42 (1H, s), 8.76 (1H, s), 10.68 (1H, tt, J=5.0 Hz), 13.77 (1H, s).

### EXAMPLE 88

3-Ethyl-8-[2-(2-tetrahydrofurilmethylamino)benzylamino]-2,3-dihydro-1H-imidazo[4,5-g]quinazoline-2-thione 2 Hydrochloride (Compound 88)

From the compound obtained in Reference Example 105, the subject compound is obtained by using approximately the same method as described in Example 22.

¹H-NMR (DMSO-d₆) δ (ppm): 1.37 (3H, t, J=7.1 Hz), 1.51-1.70 (1H, m), 1.75-2.00 (3H, m), 3.05-3.35 (2H, m), 3.60-3.80 (2H, m), 4.02-4.15 (1H, m), 4.35 (2H, q, J=7.1 Hz), 4.90 (2H, br), 5.10 (1H, br), 6.70-6.83 (2H, m), 7.15-7.25 (2H, m), 7.75 (1H, s), 8.42 (1H, s), 8.82 (1H, s), 10.50 (1H, s), 13.77(1H, s).

### EXAMPLE 89

3-Ethyl-8-[2-(2-thienyl)methylamino)benzylamino]-2,3-dihydro-1H-imidazo[4,5-g]quinazoline-2-thione 2 Hydrochloride (Compound 89)

From the compound obtained in Reference Example 107, the subject compound is obtained by using approximately the same method as described in Example 22.

¹H-NMR (DMSO-d₆) δ (ppm) 1.32 (3H, t, J=7.3 Hz), 4.36 (2H, q, J=7.3 Hz), 4.57 (2H, br), 4.85 (2H, d, J=4.6 Hz), 5.60 (1H, br), 6.60-6.78 (2H, m), 6.91-6.95 (1H, m), 7.04-7.50 (4H, m), 7.69 (1H, s), 8.39 (1H, s), 8.74 (1H, s), 10.60 (1H, t, J=4.6 Hz), 13.73(1H, s).

### EXAMPLE 90

3-Ethyl-8-[2-(2-phenylaminoethylamino)benzylamino]-2,3-dihydro-1H-imidazo[4,5-g]quinazoline-2-thione 3 Hydrochloride (Compound 90)

From the compound obtained in Reference Example 109, the subject compound is obtained by using approximately the same method as described in Example 22.

¹H-NMR (DMSO-d₆) δ (ppm): 1.33 (3H, t. J=7.0 Hz), 3.45-3.55 (4H, m), 4.36 (2H, q, J=7.0 Hz), 4.89 (2H, d, J=5.3 Hz), 5.20 (2H, br), 6.63-6.75 (2H, m), 7.12-7.36 (7H, m), 7.66 (1H, s), 8.46 (1H, s), 8.86 (1H, s), 10.70 (1H, br), 13.71 (1H, s).

### EXAMPLE 91

3-Ethyl-8-[2-(4-methoxymethylpiperidino)benzylamino]-2,3-dihydro-1H-imidazo[4,5-g]quinazoline-2-thione 2 Hydrochloride (Compound 91)

Using the compound obtained in Reference Example 112, the subject compound is obtained by using approximately the same method as described in Example 22.

¹H-NMR (DMSO-d₆) δ (ppm): 1.32 (3H, t, J=7.1 Hz,), 1.50-2.00 (5H, m), 2.60-3.20 (2H, m), 3.25-3.50 (7H, m).4.35 (2H, q, J=7.1 Hz), 5.10 (2H, br), 7.05-7.50 (4H, m), 7.77 (1H, s), 8.41 (1H, s), 8.87 (1H, s), 10.60 (1H, br), 13.76 (1H, s).

### EXAMPLE 92

8-[2-(4-Ethoxymethylpiperidino)benzylamino]-3-ethyl-2,3-dihydro-1H-imidazo[4,5-g]quinazoline-2-thione 2 Hydrochloride (Compound 92)

Using the compound obtained in Reference Example 114, the subject compound is obtained by using approximately the same method as described in Example 22.

¹H-NMR (DMSO-d₆) δ (ppm): 1.15 (3H, t, J=6.9 Hz), 1.33 (3H, t, J=7.1 Hz), 1.50-2.00 (5H, m), 3.10-3.50 (8H, m), 4.36 (2H, q, J=7.1 Hz), 5.13 (2H, br), 7.19-7.39 (4H, m), 7.77 (1H, s), 8.42 (1H, s), 8.87 (1H, s), 10.77 (1H, br), 13.73 (1H, s).

### EXAMPLE 93

8-(2-Aminobenzylamino)-3-ethyl-2,3-dihydro-1H-imidazo[4,5-g]quinazoline-2-thione 2 Hydrochloride (Compound 93)

The compound (0.58 g, 1.29 millimole) obtained in Reference Example 117 is suspended with methanol (40 ml), and 4N hydrochloric acid-ethyl acetate (10 ml) is dripped into the suspension under ice-chilled conditions. After dripping is completed, the temperature is gradually increased and the solution is stirred for 1 hour at room temperature. After the reaction is completed, the solution is concentrated, the precipitated solid is separated through filtration, washed with methanol-ether, dried, and the subject compound is obtained.

¹H-NMR (DMSO-d₆) δ (ppm): 1.32 (3H, t, J=7.1 Hz), 3.93 (2H, br), 4.36 (2H, q, J=7.1 Hz), 5.03 (2H, s), 7.14-7.19 (1H, m), 7.29-7.41 (3H, m), 7.71 (1H, s), 8.42 (1H, s), 8.91 (1H, s), 10.75 (1H.br), 13.76(1H, s).

### EXAMPLE 94

8-(2-Cyclobutylaminobenzylamino)-3-ethyl-2,3-dihydro-1H-imidazo[4,5-g]quinazoline-2-thione 2 Hydrochloride (Compound 94)

Using the compound obtained in Reference Example 119, the subject compound is obtained by using approximately the same method as described in Example 22.

¹H-NMR (DMSO-d₆) δ (ppm) -1.31 (3H, t, J=7.1 Hz), 1.74-1.85 (2H, m), 2.16-2.33 (4H, m), 4.01-4.08 (1H, m), 4.35 (2H, q, J=7.1 Hz), 4.95 (2H, d, J=4.6 Hz), 5.20 (1H, br), 6.90-7.00 (2H, m), 7.24 (1H, dd, J=7.6 Hz, 7.9 Hz), 7.32 (1H, d, J=7.6 Hz), 7.77 (1H, s), 8.45 (1H, s), 8.91 (1H, s), 10.85 (1H, t, J=4.6 Hz), 13.76 (1H, s).

### EXAMPLE 95

8-[2-exo-(2-Bicyclo[2,2,1]heptylamino)benzylamino]-3-ethyl-2,3-dihydro-1H-imidazo[4,5-g]quinazoline-2-thione 2 Hydrochloride (Compound 95)

Using the compound obtained in Reference Example 121, the subject compound is obtained by using approximately the same method as described in Example 22.

¹H-NMR (DMSO-d₆) δ (ppm): 1.08-1.21 (4H, m), 1.32 (3H, t, J=7.1 Hz), 1.49-1.60 (4H, m), 1.70-1.78 (1H, m), 2.25-2.32 (2H, m), 3.34-3.38 (1H, m), 4.36 (2H, q, J=7.1 Hz), 4.92 (2H, d, J=4.6 Hz), 6.79-6.85 (2H, m), 7.21 (1H, dd, J=7.6 Hz, 7.6 Hz), 7.28 (1H, d, J=7.3 Hz), 7.72 (1H.s), 8.38 (1H, s), 8.90 (1H, s). 10.6 8 (1H, t, J=4.6 Hz), 13.73(1H, s).

### EXAMPLE 96

8-[2-(4-Ethoxycarbonyl-1-piperazinyl)benzylamino]-3-ethyl-2,3-dihydro-1H-imidazo[4,5-g]quinazoline-2-thione 2 Hydrochloride (Compound 96)

Using the compound obtained in Reference Example 71, the subject compound is obtained by using approximately the same method as described in Example 22.

¹H-NMR-(As free base, DMSO-d₆) δ (ppm): 1.24 (3H, t, J=7.1 Hz), 1.34 (3H, t, J=7.1 Hz), 2.88-2.93 (4H, m), 3.50-3.60 (4H, m), 4.09 (2H, q, J=7.1 Hz), 4.36 (2H, q, J=7.1 Hz), 4.93 (2H, d, J=5.3 Hz), 7.02 (1H, dd, J=7.3 Hz, 7.3 Hz), 7.13 (1H, d, J=7.3 Hz), 7.20-7.27 (2H, m), 7.56 (1H, s), 8.14 (1H, s), 8.42 (1H, s), 8.85 (1H, t, J=5.3 Hz), 13.22(1H, s).

It will be understood that various changes and modifications can be made in the details of procedure, formulation and use without departing from the spirit of the invention, especially as defined in the following claims.

We claim:

1. A method of treating a mammal having precancerous lesions comprising administering a pharmacologically effective amount of a compound of Formula I or pharmaceutically acceptable salt thereof: 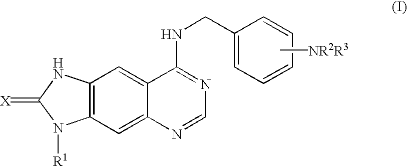
wherein
R′ is selected from the group consisting of hydrogen, a substituted or unsubstituted lower alkyl, a substituted or unsubstituted cycloalkyl, a substituted or unsubstituted bicycloalkyl, a substituted or unsubstituted tricycloalkyl, a substituted or unsubstituted benzocycloalkenyl, a substituted or unsubstituted lower alkenyl, a substituted or unsubstituted aralkyl group, a substituted or unsubstituted aryl group, or a substituted or unsubstituted hetero-aryl group;
R² is selected from the group consisting of hydrogen, a substituted or unsubstituted lower alkyl, a substituted or unsubstituted cycloalkyl, a substituted or unsubstituted bicycloalkyl, a substituted or unsubstituted tricycloalkyl, a substituted or unsubstituted benzocycloalkenyl, a substituted or unsubstituted lower alkenyl, a substituted or unsubstituted aralkyl group, a substituted or unsubstituted aryl group, or a substituted or unsubstituted heteroaryl group;
R³ is selected from the group consisting of hydrogen, a substituted or unsubstituted lower alkyl, a substituted or unsubstituted cycloalkyl, a substituted or unsubstituted bicycloalkyl, a substituted or unsubstituted tricycloalkyl, a substituted or unsubstituted lower alkenyl, a substituted or unsubstituted aralkyl, a substituted or unsubstituted aryl group or a substituted or unsubstituted hetero-aryl group; or R² and R³ may, together with N to which they are attached, form a substituted or unsubstituted heterocyclic group; and
X represents O or S.

2. The method of claim 1, wherein R³ is a hydrogen.

3. The method of claim 1 wherein R² is selected from a substituted or unsubstituted lower alkyl and R³ is a lower hydroxy alkyl.

4. The method of claim 1 wherein R² and R³ together with N to which they are attached form a substituted or unsubstituted heterocyclic ring.

5. The method of claim 1 wherein R¹ is selected from a substituted or unsubstituted lower alkyl, and R³.

6. The method of claim 1 wherein X is sulfur.

7. The method of claim 6, wherein R² represents a substituted or non-substituted lower alkyl and R³.

8. The method of claim 7 wherein R² is selected from the group consisting of a hydrogen, a substituted or unsubstituted lower alkyl, a substituted or unsubstituted cycloalkyl, a substituted or un substituted bicycloalkyl, a substituted or unsubstituted tricycloalkyl, a substituted or unsubstituted lower alkenyl, or a substituted or unsubstituted aralkyl, R³ represents a hydrogen, a substituted or unsubstituted lower alkyl, a substituted or unsubstituted cycloalkyl, a substituted or unsubstituted bicycloalkyl, a substituted or unsubstituted tricycloalkyl, a substituted or unsubstituted lower alkenyl, or a substituted or un substituted aralkyl.

9. The method of claim 7 wherein R² and R³ combine together with the N to which they are attached to form a substituted or unsubstituted heterocyclic ring.

10. A method for inhibiting the growth of neoplastic cells comprising exposing the cells to a growth inhibiting effective amount of a compound of Formula I or pharmaceutically acceptable salt thereof: 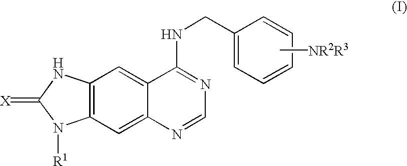
wherein
R′ is selected from the group consisting of hydrogen, a substituted or unsubstituted lower alkyl a substituted or unsubstituted cycloalkyl, a substituted or unsubstituted bicycloalkyl, a substituted or unsubstituted tricycloalkyl, a substituted or unsubstituted benzocycloalkenyl, a substituted or unsubstituted lower alkenyl, a substituted or unsubstituted aralkyl group, a substituted or unsubstituted aryl group, or a substituted or unsubstituted hetero-aryl group;
R² is selected from the group consisting of hydrogen, a substituted or unsubstituted lower alkyl, a substituted or unsubstituted cycloalkyl, a substituted or unsubstituted bicycloalkyl, a substituted or unsubstituted tricycloalkyl, a substituted or unsubstituted benzocycloalkenyl, a substituted or unsubstituted lower alkenyl, a substituted or unsubstituted aralkyl group, a substituted or unsubstituted aryl group, or a substituted or unsubstituted heteroaryl group;
R³ is selected from the group consisting of hydrogen, a substituted or unsubstituted lower alkyl, a substituted or unsubstituted cycloalkyl, a substituted or unsubstituted bicycloalkyl, a substituted or unsubstituted tricycloalkyl, a substituted or unsubstituted lower alkenyl, a substituted or unsubstituted aralkyl, a substituted or unsubstituted aryl group or a substituted or unsubstituted hetero-aryl group; or R² and R³ may, together with N to which they are attached, form a substituted or unsubstituted heterocyclic group; and
X represents O or S.

11. The method of claim 10, wherein R¹ is a hydrogen.

12 The method of claim 10 wherein R² is selected from a substituted or unsubstituted lower alkyl and R³ is a lower hydroxy alkyl.

13. The method of claim 10 wherein R² and R³ together with N to which they are attached form a substituted or unsubstituted heterocyclic ring.

14. The method of claim 10 wherein R¹ is selected from a substituted or unsubstituted lower alkyl, and R³.

15. The method of claim 10 wherein X is sulfur.

16. The method of claim 15, wherein R² represents a substituted or non-substituted lower alkyl and R³.

17. The method of claim 16 wherein R² is selected from the group consisting of a hydrogen, a substituted or unsubstituted lower alkyl, a substituted or unsubstituted cycloalkyl, a substituted or unsubstituted bicycloalkyl, a substituted or unsubstituted tricycloalkyl, a substituted or unsubstituted lower alkenyl, or a substituted or unsubstituted aralkyl, R³ represents a hydrogen, a substituted or unsubstituted lower alkyl, a substituted or unsubstituted cycloalkyl, a substituted or unsubstituted bicycloalkyl, a substituted or unsubstituted tricycloalkyl, a substituted or unsubstituted lower alkenyl, or a substituted or unsubstituted aralkyl. The method of claim 7 wherein R² and R³ combine together with the N to which they are attached to form a substituted or unsubstituted heterocyclic ring.

18. A method for regulating apoptosis in human cells comprising exposing said cells to an effective amount of a compound of the formula: 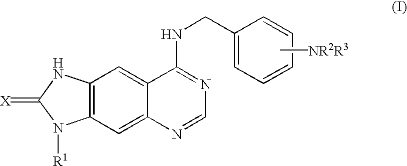
wherein
R′ is selected from the group consisting of hydrogen, a substituted or unsubstituted lower alkyl, a substituted or unsubstituted cycloalkyl, a substituted or unsubstituted bicycloalkyl, a substituted or unsubstituted tricycloalkyl, a substituted or unsubstituted benzocycloalkenyl, a substituted or unsubstituted lower alkenyl, a substituted or unsubstituted aralkyl group, a substituted or unsubstituted aryl group, or a substituted or unsubstituted hetero-aryl group;
R² is selected from the group consisting of hydrogen, a substituted or unsubstituted lower alkyl, a substituted or unsubstituted cycloalkyl, a substituted or unsubstituted bicycloalkyl, a substituted or unsubstituted tricycloalkyl, a substituted or unsubstituted benzocycloalkenyl, a substituted or unsubstituted lower alkenyl, a substituted or unsubstituted aralkyl group, a substituted or unsubstituted aryl group, or a substituted or unsubstituted heteroaryl group;
R³ is selected from the group consisting of hydrogen, a substituted or unsubstituted lower alkyl, a substituted or unsubstituted cycloalkyl, a substituted or unsubstituted bicycloalkyl, a substituted or unsubstituted tricycloalkyl, a substituted or unsubstituted lower alkenyl, a substituted or unsubstituted aralkyl, a substituted or unsubstituted aryl group or a substituted or unsubstituted hetero-aryl group; or R² and R³ may, together with N to which they are attached, form a substituted or unsubstituted heterocyclic group; and
X represents O or S.

19. The method of claim 18 wherein R³ is a hydrogen.

20. The method of claim 18 wherein R² is selected from a substituted or unsubstituted lower alkyl and R³ is a lower hydroxy alkyl.

21. The method of claim 18 wherein R² and R³ together with N to which they are attached form a substituted or unsubstituted heterocyclic ring.

22. The method of claim 18 wherein R¹ is selected from a substituted or unsubstituted lower alkyl, and R³.

23. The method of claim 18 wherein X is sulfur.

24. The method of claim 23, wherein R² represents a substituted or non-substituted lower alkyl and R³.

25. The method of claim 24 wherein R² is selected from the group consisting of a hydrogen, a substituted or unsubstituted lower alkyl, a substituted or unsubstituted cycloalkyl, a substituted or unsubstituted bicycloalkyl, a substituted or unsubstituted tricycloalkyl, a substituted or unsubstituted lower alkenyl, or a substituted or unsubstituted aralkyl, R represents a hydrogen, a substituted or unsubstituted lower alkyl, a substituted or unsubstituted cycloalkyl, a substituted or unsubstituted bicycloalkyl, a substituted or unsubstituted tricycloalkyl, a substituted or unsubstituted lower alkenyl, or a substituted or unsubstituted aralkyl. The method of claim 7 wherein R² and R³ combine together with the N to which they are attached to form a substituted or unsubstituted heterocyclic ring.
